(19) United States
(12) Patent Application Publication
(10) Pub. No.: US 2003/0219793 A1
(43) Pub. Date: Nov. 27, 2003

(54) High bone mass gene of 11q13.3

(76) Inventors: John P. Carulli, Southboro, MA (US); Robert R. Recker, Omaha, NE (US); Mark L. Johnson, Omaha, NE (US); Randall D. Little, Newtonville, MA (US)

Correspondence Address: Estelle J. Tsevdos, Esq., KENYON & KENYON, One Broadway, New York, NY, 10004, US

(21) Appl. No.: 10/374,979

(22) Filed: Feb. 28, 2003

Related U.S. Application Data
(63) Continuation of application No. 10/240,851, filed on Oct. 4, 2002.
Application No. 10/240,851 is a 371 of international application No. PCT/US00/16951, filed on Jun. 21, 2000.
Application No. 10/240,851 is a 371 of international application No. 09/578,900, filed on May 26, 2000.

(51) Int. Cl. C12Q 1/68; C07H 21/04; C12N 9/64; C12P 21/02; C12N 5/06; C07K 16/40

(52) U.S. Cl. 435/6; 435/69.1; 435/226; 435/320.1; 435/325; 536/23.2; 530/388.26

(57) ABSTRACT

The present invention relates to methods and materials used to isolate and detect a high bone mass gene and a corresponding wild-type gene, and mutants thereof. The present invention also relates to the high bone mass gene, the corresponding wild-type gene, and mutants thereof. The genes identified in the present invention are implicated in bone development and in focal adhesion signaling. The invention also provides nucleic acids, including coding sequences, oligonucleotide primers and probes, proteins, cloning vectors, expression vectors, transformed hosts, methods of developing pharmaceutical compositions, methods of identifying molecules involved in bone development, and methods of diagnosing and treating diseases involved in bone development. In preferred embodiments, the present invention is directed to methods for treating, diagnosing and preventing osteoporosis.

### FIELD OF THE INVENTION

The present invention relates generally to the field of genetics, genomics and molecular biology. More particularly, the invention relates to methods and materials used to isolate, detect and sequence a high bone mass gene and corresponding wild-type gene, and mutants thereof. The present invention also relates to the high bone mass gene, the corresponding wild-type gene, and mutants thereof. The genes identified in the present invention are implicated in the ontology and physiology of bone development. The invention also provides nucleic acids, proteins, cloning vectors, expression vectors, transformed hosts, methods of developing pharmaceutical compositions, methods of identifying molecules involved in bone development, and methods of diagnosing and treating diseases involved in bone development. In preferred embodiments, the present invention is directed to methods for treating, diagnosing, preventing and screening for normal and abnormal conditions of bone, including metabolic bone diseases such as osteoporosis.

### BACKGROUND OF THE INVENTION

Two of the most common types of osteoporosis are postmenopausal and senile osteoporosis. Osteoporosis affects men as well as women, and, taken with other abnormalities of bone, presents an ever-increasing health risk for an aging population. The most common type of osteoporosis is that associated with menopause. Most women lose between 20-60% of the bone mass in the trabecular compartment of the bone within 3-6 years after the cessation of menses. This rapid loss is generally associated with an increase of bone resorption and formation. However, the resorptive cycle is more dominant and the result is a net loss of bone mass. Osteoporosis is a common and serious disease among postmenopausal women. There are an estimated 25 million women in the United States alone who are afflicted with this disease. The results of osteoporosis are both personally harmful, and also account for a large economic loss due to its chronicity and the need for extensive and long-term support (hospitalization and nursing home care) from the disease sequelae. This is especially true in more elderly patients. Additionally, while osteoporosis is generally not thought of as a life-threatening condition, a 20-30% mortality rate is related to hip fractures in elderly women. A large percentage of this mortality rate can be directly associated with postmenopausal osteoporosis.

The most vulnerable tissue in the bone to the effects of postmenopausal osteoporosis is the trabecular bone. This tissue is often referred to as spongy bone and is particularly concentrated near the ends of the bone near the joints and in the vertebrae of the spine. The trabecular tissue is characterized by small structures which inter-connect with each other as well as the more solid and dense cortical tissue which makes up the outer surface and central shaft of the bone. This crisscross network of trabeculae gives lateral support to the outer cortical structure and is critical to the biomechanical strength of the overall structure. In postmenopausal osteoporosis, it is primarily the net resorption and loss of the trabeculae which lead to the failure and fracture of the bone. In light of the loss of the trabeculae in postmenopausal women, it is not surprising that the most common fractures are those associated with bones which are highly dependent on trabecular support, e.g., the vertebrae, the neck of the femur, and the forearm. Indeed, hip fracture, Colle's fractures, and vertebral crush fractures are indicative of postmenopausal osteoporosis.

One of the earliest generally accepted methods for treatment of postmenopausal osteoporosis was estrogen replacement therapy. Although this therapy frequently is successful, patient compliance is low, primarily due to the undesirable side-effects of chronic estrogen treatment. Frequently cited side-effects of estrogen replacement therapy include reinitiation of menses, bloating, depression, and fear of breast or uterine cancer. In order to limit the known threat of uterine cancer in those women who have not undergone a hysterectomy, a protocol of estrogen and progestin cyclic therapy is often employed. This protocol is similar to that which is used in birth control regimens, and often is not tolerated by women because of the side-effects characteristic of progestin. More recently, certain antiestrogens, originally developed for the treatment of breast cancer, have been shown in experimental models of postmenopausal osteoporosis to be efficacious. Among these agents is raloxifene (See, U.S. Pat. No. 5,393,763, and Black et al, J. Clin. Invest., 93:63-69 (1994)). In addition, tamoxifene, a widely used clinical agent for the treatment of breast cancer, has been shown to increase bone mineral density in post menopausal women suffering from breast cancer (Love et al, N. Engl. J. Med., 326:852-856 (1992)).

Another therapy for the treatment of postmenopausal osteoporosis is the use of calcitonin. Calcitonin is a naturally occurring peptide which inhibits bone resorption and has been approved for this use in many countries (Overgaard et al, Br. Med. J, 305:556-561 (1992)). The use of calcitonin has been somewhat limited, however. Its effects are very modest in increasing bone mineral density and the treatment is very expensive. Another therapy for the treatment of postmenopausal osteoporosisis the use of bis-phosphonates. These compounds were originally developed for use in Paget's disease and malignant hypercalcemia. They have been shown to inhibit bone resorption. Alendronate, one compound of this class, has been approved for the treatment of postmenopausal osteoporosis. These agents may be helpful in the treatment of osteoporosis, but these agents also have potential liabilities which include osteomalacia, extremely long half-life in bone (greater than 2 years), and possible “frozen bone syndrome,” e.g., the cessation of normal bone remodeling.

Senile osteoporosis is similar to postmenopausal osteoporosis in that it is marked by the loss of bone mineral density and resulting increase in fracture rate, morbidity, and associated mortality. Generally, it occurs in later life, i.e., after 70 years of age. Historically, senile osteoporosis has been more common in females, but with the advent of a more elderly male population, this disease is becoming a major factor in the health of both sexes. It is not clear what, if any, role hormones such as testosterone or estrogen have in this disease, and its etiology remains obscure. Treatment of this disease has not been very satisfactory. Hormone therapy, estrogen in women and testosterone in men, has shown equivocal results; calcitonin and bis-phosphonates may be of some utility.

The peak mass of the skeleton at maturity is largely under genetic control. Twin studies have shown that the variance in bone mass between adult monozygotic twins is smaller than between dizygotic twins (Slemenda et al, J. Bone Miner. Res., 6:561-567 (1991); Young et al, J. Bone Miner. Res., 6:561-567 (1995); Pocock et al, J. Clin. Invest., 80:706-710 (1987); Kelly et al, J. Bone Miner. Res., 8:11-17 (1993)), and it has been estimated that up to 60% or more of the variance in skeletal mass is inherited (Krall et al, J. Bone Miner. Res., 10:S367 (1993)). Peak skeletal mass is the most powerful determinant of bone mass in elderly years (Hui et al, Ann. Int. Med., 111:355-361 (1989)), even though the rate of age-related bone loss in adult and later life is also a strong determinant (Hui et al, Osteoporosis Int., 1:30-34 (1995)). Since bone mass is the principal measurable determinant of fracture risk, the inherited peak skeletal mass achieved at maturity is an important determinant of an individual's risk of fracture later in life. Thus, study of the genetic basis of bone mass is of considerable interest in the etiology of fractures due to osteoporosis.

Recently, a strong interest in the genetic control of peak bone mass has developed in the field of osteoporosis. The interest has focused mainly on candidate genes with suitable polymorphisms to test for association with variation in bone mass within the normal range, or has focused on examination of genes and gene loci associated with low bone mass in the range found in patients with osteoporosis. The vitamin D receptor locus (VDR) (Morrison et al, Nature, 367:284-287 (1994)), PTH gene (Howard et al, J. Clin. Endocrinol. Metab., 80:2800-2805 (1995); Johnson et al, J. Bone Miner. Res., 8:11-17 (1995); Gong et al, J. Bone Miner. Res., 10:S462 ((1995)) and the estrogen receptor gene (Hosoi et al, J. Bone Miner. Res., 10:S170 (1995); Morrison et al, Nature, 367:284-287 (1994)) have figured most prominently in this work. These studies are difficult because bone mass (the phenotype) is a continuous, quantitative, polygenic trait, and is confounded by environmental factors such as nutrition, co-morbid disease, age, physical activity, and other factors. Also, this type of study design requires large numbers of subjects. In particular, the results of VDR studies to date have been confusing and contradictory (Garnero et al, J. Bone Miner. Res., 10: 1283-1288 (1995); Eisman et al, J. Bone. Miner. Res., 10:1289-1293 (1995); Peacock, J. Bone Miner. Res., 10:1294-1297 (1995)). Furthermore, the work thus far has not shed much light on the mechanism(s) whereby the genetic influences might exert their effect on bone mass.

While it is well known that peak bone mass is largely determined by genetic rather than environmental factors, studies to determine the gene loci (and ultimately the genes) linked to variation in bone mass are difficult and expensive. Study designs which utilize the power of linkage analysis, e.g., sib-pair or extended family, are generally more informative than simple association studies, although the latter do have value. However, genetic linkage studies involving bone mass are hampered by two major problems. The first problem is the phenotype, as discussed briefly above. Bone mass is a continuous, quantitative trait, and establishing a discrete phenotype is difficult. Each anatomical site for measurement maybe influenced by several genes, many of which may be different from site to site. The second problem is the age component of the phenotype. By the time an individual can be identified as having low bone mass, there is a high probability that their parents or other members of prior generations will be deceased and therefore unavailable for study, and younger generations may not have even reached peak bone mass, making their phenotyping uncertain for genetic analysis.

Regardless, linkage analysis can be used to find the location of a gene causing a hereditary “disorder” and does not require any knowledge of the biochemical nature of the disorder, i.e., a mutated protein that is believed to cause the disorder does not need to be known. Traditional approaches depend on assumptions concerning the disease process that might implicate a known protein as a candidate to be evaluated. The genetic localization approach using linkage analysis can be used to first find the general chromosomal region in which the defective gene is located and then to gradually reduce the size of the region in order to determine the location of the specific mutated gene as precisely as possible. After the gene itself is discovered within the candidate region, the messenger RNA and the protein are identified and, along with the DNA, are checked for mutations.

The genetic localization approach has practical implications since the location of the disease can be used for prenatal diagnosis even before the altered gene that causes the disease is found. Linkage analysis can enable families, even many of those that do not have a sick child, to know whether they are carriers of a disease gene and to evaluate the condition of an unborn child through molecular diagnosis. The transmission of a disease within families, then, can be used to find the defective gene. As used herein, reference to “high bone mass” (HBM) is analogous to reference to a disease state, although from a practical standpoint high bone mass can actually help a subject avoid the disease known as osteoporosis.

Linkage analysis is possible because of the nature of inheritance of chromosomes from parents to offspring. During meiosis, the two parental homologues pair to guide their proper separation to daughter cells. While they are lined up and paired, the two homologues exchange pieces of the chromosomes, in an event called “crossing over” or “recombination.” The resulting chromosomes are chimeric, that is, they contain parts that originate from both parental homologues. The closer together two sequences are on the chromosome, the less likely that a recombination event will occur between them, and the more closely linked they are. In a linkage analysis experiment, two positions on the chromosomes are followed from one generation to the next to determine the frequency of recombination between them. In a study of an inherited disease, one of the chromosomal positions is marked by the disease gene or its normal counterpart, i.e., the inheritance of the chromosomal region can be determined by examining whether the individual displays symptoms of the disorder or not. The other position is marked by a DNA sequence that shows natural variation in the population such that the two homologues can be distinguished based on the copy of the “marker” sequence that they possess. In every family, the inheritance of the genetic marker sequence is compared to the inheritance of the disease state. If, within a family carrying an autosomal dominant disorder such as high bone mass, every affected individual carries the same form of the marker and all the unaffected individuals carry at least one different form of the marker, there is a great probability that the disease gene and the marker are located close to each other. In this way, chromosomes may be systematically checked with known markers and compared to the disease state. The data obtained from the different families is combined, and analyzed together by a computer using statistical methods. The result is information indicating the probability of linkage between the genetic marker and the disease allowing different distances between them. A positive result can mean that the disease is very close to the marker, while a negative result indicates that it is far away on that chromosome, or on an entirely different chromosome.

Linkage analysis is performed by typing all members of the affected family at a given marker locus and evaluating the co-inheritance of a particular disease state with the marker probe, thereby determining how often the two of them are co-inherited. The recombination frequency can be used as a measure of the genetic distance between two gene loci. A recombination frequency of 1% is equivalent to 1 map unit, or 1 centiMorgan (cM), which is roughly equivalent to 1,000 kb of DNA. This relationship holds up to frequencies of about 20% or 20 cM.

The entire human genome is 3,300 cM long. In order to find an unknown disease gene within 5-10 cM of a marker locus, the whole human genome can be searched with roughly 330 informative marker loci spaced at approximately 10 cM intervals (Botstein et al, Am. J. Hum. Genet., 32:314-331 (1980)). The reliability of linkage results is established by using a number of statistical methods. The method most commonly used for the analysis of linkage in humans is the LOD score method (Morton, Prog. Clin. Biol. Res., 147:245-265 (1984), Morton et al, Am. J. Hum. Genet., 38:868-883 (1986)) which was incorporated into the computer program LIPED by Ott, Am. J. Hum. Genet., 28:528-529 (1976). LOD scores are the logarithm of the ratio of the likelihood that two loci are linked at a given distance to that they are not linked (>50 cM apart). The advantage of using logarithmic values is that they can be summed among families with the same disease. This becomes necessary given the relatively small size of human families.

By convention, a total LOD score greater than +3.0 (that is, odds of linkage at the specified recombination frequency being 1000 times greater than odds of no linkage) is considered to be significant evidence for linkage at that particular recombination frequency. A total LOD score of less than −2.0 (that is, odds of no linkage being 100 times greater than odds of linkage at the specified frequency) is considered to be strong evidence that the two loci under consideration are not linked at that particular recombination frequency. Until recently, most linkage analyses have been performed on the basis of two-point data, which is the relationship between the disorder under consideration and a particular genetic marker. However, as a result of the rapid advances in mapping the human genome over the last few years, and concomitant improvements in computer methodology, it has become feasible to carry out linkage analyses using multi-point data. Multi-point analysis provide a simultaneous analysis of linkage between the disease and several linked genetic markers, when the recombination distance among the markers is known.

Multi-point analysis is advantageous for two reasons. First, the informativeness of the pedigree is usually increased. Each pedigree has a certain amount of potential information, dependent on the number of parents heterozygous for the marker loci and the number of affected individuals in the family. However, few markers are sufficiently polymorphic as to be informative in all those individuals. If multiple markers are considered simultaneously, then the probability of an individual being heterozygous for at least one of the markers is greatly increased. Second, an indication of the position of the disease gene among the markers may be determined. This allows identification of flanking markers, and thus eventually allows isolation of a small region in which the disease gene resides. Lathrop et al, Proc. Natl. Acad. Sci. USA, 81:3443-3446 (1984) have written the most widely used computer package, LINKAGE, for multi-point analysis.

There is a need in the art for identifying the gene associated with a high bone mass phenotype. The present invention is directed to this, as well as other, important ends.

### SUMMARY OF THE INVENTION

The present invention describes the Zmax1 gene and the HBM gene on chromosome 11q13.3 by genetic linkage and mutation analysis. The use of additional genetic markers linked to the genes has aided this discovery. By using linkage analysis and mutation analysis, persons predisposed to HBM may be readily identified. Cloning methods using Bacterial Artificial Chromosomes have enabled the inventors to focus on the chromosome region of 11q13.3 and to accelerate the sequencing of the autosomal dominant gene. In addition, the invention identifies the Zmax1 gene and the HBM gene, and identifies the guanine-to-thymine polymorphism mutation at position 582 in the Zmax1 gene that produces the HBM gene and the HBM phenotype.

The present invention identifies the Zmax1 gene and the HBM gene, which can be used to determine if people are predisposed to HBM and, therefore, not susceptible to diseases characterized by reduced bone density, including, for example, osteoporosis, or are predisposed and susceptible to diseases characterized by abnormally high bone density, such as, for example, osteoporosis. Older individuals carrying the HBM gene express the HBM protein, and, therefore, do not develop osteoporosis. In other words, the HBM gene is a suppressor of osteoporosis. This in vivo observation is a strong evidence that treatment of normal individuals with the HBM gene or protein, or fragments thereof, will ameliorate osteoporosis.

Moreover, such treatment will be indicated in the treatment of bone lesions, particularly bone fractures, for bone remodeling in the healing of such lesions. For example, persons predisposed to or suffering from stress fractures (i.e., the accumulation of stress-induced microfractures, eventually resulting in a true fracture through the bone cortex) may be identified and/or treated by means of the invention. Moreover, the methods and compositions of the invention will be of use in the treatment of secondary osteoporosis, where the course of therapy involves bone remodeling, such as endocrine conditions accompanying corticosteroid administration, hyperthyroidism, hypogonadism, hematologic malignancies, malabsorption and alcoholism, as well as disorders associated with vitamin D and/or phosphate metabolism, such as osteomalacia and rickets, and diseases characterized by abnormal or disordered bone remodeling, such as Paget's disease, and in neoplasms of bone, which may be benign or malignant.

In various embodiments, the present invention is directed to nucleic acids, proteins, vectors, and transformed hosts of HBM and Zmax1.

Additionally, the present invention is directed to applications of the above embodiments of the invention including, for example, gene therapy, pharmaceutical development, and diagnostic assays for bone development disorders. In preferred embodiments, the present invention is directed to methods for treating, diagnosing, preventing and screening for osteoporosis.

These and other aspects of the present invention are described in more detail below.

### BRIEF DESCRIPTION OF THE FIGURES

FIGS. 1A and 1B show the pedigree of the individuals used in the genetic linkage studies. Under each individual is an ID number, the z-score for spinal BMD, and the allele calls for the critical markers on chromosome 11. Solid symbols represent “affected” individuals. Symbols containing “N” are “unaffected” individuals. DNA from 37 individuals was genotyped. Question marks denote unknown genotypes or individuals who were not genotyped.

FIGS. 2A and 2B depict the BAC/STS content physical map of the HBM region in 11q13.3. STS markers derived from genes, ESTs, microsatellites, random sequences, and BAC endsequences are denoted above the long horizontal line. For markers that are present in GDB the same nomenclature has been used. Locus names (D11S####) are listed in parentheses after the primary name if available. STSs derived from BAC endsequences are listed with the BAC name first followed by L or R for the left and right end of the clone, respectively. The two large arrows indicate the genetic markers that define the HBM critical region. The horizontal lines below the STSs indicate BAC clones identified by PCR-based screening of a nine-fold coverage BAC library. Open circles indicate that the marker did not amplify the corresponding BAC library address during library screening. Clone names use the following convention: B for BAC, the plate, row and column address, followed by -H indicating the HBM project (i.e., B36F16-H).

FIGS. 3A-3F show the genomic structure of Zmax1 with flanking intron sequences. Translation is initiated by the underlined “ATG” in exon 1. The site of the polymorphism in the HBM gene is in exon 3 and is represented by the underlined “G,” whereby this nucleotide is a “T” in the HBM gene. The 3′ untranslated region of the mRNA is underlined within exon 23 (exon 1, SEQ ID NO:40; exon 2, SEQ ID NO:41; exon 3, SEQ ID NO:42; exon 4, SEQ ID NO:43; exon 5, SEQ ID NO:44; exon 6, SEQ ID NO:45; exon 7, SEQ ID NO:46; exon 8, SEQ ID NO:47; exon 9, SEQ ID NO:48; exon 10, SEQ ID NO:49; exon 11, SEQ ID NO:50; exon 12, SEQ ID NO:51; exon 13, SEQ ID NO:52; exon 14, SEQ ID _NO:53; exon 15, SEQ ID NO:54; exon 16, SEQ ID NO:55; exon 17, SEQ ID NO:56; exon 18, SEQ ID NO:57; exon 19, SEQ ID NO:58; exon 20, SEQ ID NO:59; exon 21, SEQ ID NO:60; exon 22, SEQ ID NO:61; and exon 23; SEQ ID NO:62).

FIG. 4 shows the domain organization of Zmax1, including the YWTD spacers, the extracellular attachment site, the binding site for LDL and calcium, the cysteine-rich growth factor repeats, the transmembrane region, the ideal PEST region with the CK-II phosphorylation site and the internalization domain. FIG. 4 also shows the site of the glycine to valine change that occurs in the HBM protein. The signal peptide is located at amino acids 1-22, the extracellular domain is located at amino acids 23-1385, the transmembrane segment is located at amino acids 1386-1413, and the cytoplasmic domain is located at amino acids 1414-1615.

FIG. 5 is a schematic illustration of the BAC contigs B527D12 and B200E21 in relation to the HBM gene.

FIGS. 6A-6J are the nucleotide and amino acid sequences of the wild-type gene, Zmax1. The location for the base pair substitution at nucleotide 582, a guanine to thymine, is underlined. This allelic variant is the HBM gene. The HBM gene encodes for a protein with an amino acid substitution of glycine to valine at position 171. The 5′ untranslated region (UTR) boundaries bases 1 to 70, and the 3′ UTR boundaries bases 4916-5120.

FIGS. 7A and 7B are northern blot analyses showing the expression of Zmax1 in various tissues.

FIG. 8 is a PCR product analysis.

FIG. 9 is allele specific oligonucleotide detection of the Zmax1 exon 3 mutation.

FIG. 10 is the cellular localization of mouse Zmax1 by in situ hybridization at 100× magnification using sense and antisense probes.

FIG. 11 is the cellular localization of mouse Zmax1 by in situ hybridization at 400× magnification using sense and antisense probes.

FIG. 12 is the cellular localization of mouse Zmax1 by in situ hybridization of osteoblasts in the endosteum at 400× magnification using sense and antisense probes.

FIG. 13 shows antisense inhibition of Zmax1 expression in MC-3T3 cells.

FIG. 14 shows a Zmax1 Exon3 Allele Specific Oligonucleotide (ASO) assay which illustrates the rarity of the HBM1 allele (right panels; T-specific oligo; 58° C. Wash) as compared to the wild-type Zmax1 allele (left panels, G-specific oligo; 55° C. Wash). The positive spots appearing in the right panels were positive controls.

FIG. 15 depicts a model representing the potential role of Zmax1 in focal adhesion signaling.

### DETAILED DESCRIPTION OF THE INVENTION

To aid in the understanding of the specification and claims, the following definitions are provided.

“Gene” refers to a DNA sequence that encodes through its template or messenger RNA a sequence of amino acids characteristic of a specific peptide. The term “gene” includes intervening, non-coding regions, as well as regulatory regions, and can include 5′ and 3′ ends.

“Gene sequence” refers to a DNA molecule, including both a DNA molecule which contains a non-transcribed or non-translated sequence. The term is also intended to include any combination of gene(s), gene-fragment(s), non-transcribed sequence(s) or non-translated sequence(s) which are present on the same DNA molecule.

The sequences of the present invention may be derived from a variety of sources including DNA, cDNA, synthetic DNA, synthetic RNA or combinations thereof. Such sequences may comprise genomic DNA which may or may not include naturally occurring introns. Moreover, such genomic DNA may be obtained in association with promoter regions or poly (A) sequences. The sequences, genomic DNA or cDNA may be obtained in any of several ways. Genomic DNA can be extracted and purified from suitable cells by means well known in the art. Alternatively, mRNA can be isolated from a cell and used to produce cDNA by reverse transcription or other means.

“cDNA” refers to complementary or copy DNA produced from an RNA template by the action of RNA-dependent DNA polymerase (reverse transcriptase). Thus, a “cDNA clone” means a duplex DNA sequence complementary to an RNA molecule of interest, carried in a cloning vector or PCR amplified. This term includes genes from which the intervening sequences have been removed.

“Recombinant DNA” means a molecule that has been recombined by in vitro splicing cDNA or a genomic DNA sequence.

“Cloning” refers to the use of in vitro recombination techniques to insert a particular gene or other DNA sequence into a vector molecule. In order to successfully clone a desired gene, it is necessary to use methods for generating DNA fragments, for joining the fragments to vector molecules, for introducing the composite DNA molecule into a host cell in which it can replicate, and for selecting the clone having the target gene from amongst the recipient host cells.

“cDNA library” refers to a collection of recombinant DNA molecules containing cDNA inserts which together comprise the entire genome of an organism. Such a cDNA library can be prepared by methods known to one skilled in the art and described by, for example, Cowell and Austin, “cDNA Library Protocols,” Methods in Molecular Biology (1997). Generally, RNA is first isolated from the cells of an organism from whose genome it is desired to clone a particular gene.

“Cloning vehicle” refers to a plasmid or phage DNA or other DNA sequence which is able to replicate in a host cell. The cloning vehicle is characterized by one or more endonuclease recognition sites at which such DNA sequences may be cut in a determinable fashion without loss of an essential biological function of the DNA, which may contain a marker suitable for use in the identification of transformed cells.

“Expression control sequence” refers to a sequence of nucleotides that control or regulate expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Expression control sequences will vary depending on whether the vector is designed to express the operably linked gene in a prokaryotic or eukaryotic host, and may contain transcriptional elements such as enhancer elements, termination sequences, tissue-specificity elements and/or translational initiation and termination sites.

“Expression vehicle” refers to a vehicle or vector similar to a cloning vehicle but which is capable of expressing a gene which has been cloned into it, after transformation into a host. The cloned gene is usually placed under the control of (i.e., operably linked to) an expression control sequence.

“Operator” refers to a DNA sequence capable of interacting with the specific repressor, thereby controlling the transcription of adjacent gene(s).

“Promoter” refers to a DNA sequence that can be recognized by an RNA polymerase. The presence of such a sequence permits the RNA polymerase to bind and initiate transcription of operably linked gene sequences.

“Promoter region” is intended to include the promoter as well as other gene sequences which may be necessary for the initiation of transcription. The presence of a promoter region is sufficient to cause the expression of an operably linked gene sequence.

“Operably linked” means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

“Prokaryote” refers to all organisms without a true nucleus, including bacteria.

“Eukaryote” refers to organisms and cells that have a true nucleus, including mammalian cells.

“Host” includes prokaryotes and eukaryotes, such as yeast and filamentous fungi, as well as plant and animal cells. The term includes an organism or cell that is the recipient of a replicable expression vehicle.

“Fragment” of a gene refers to any variant of the gene that possesses the biological activity of that gene.

“Variant” refers to a gene that is substantially similar in structure and biological activity or immunological characteristics to either the entire gene or to a fragment of the gene. Provided that the two genes possess a similar activity, they are considered variant as that term is used herein even if the sequence of amino acid residues is not identical.

“Amplification of nucleic acids” refers to methods such as polymerase chain reaction (PCR), ligation amplification (or ligase chain reaction, LCR) and amplification methods based on the use of Q-beta replicase. These methods are well known in the art and described, for example, in U.S. Pat. Nos. 4,683,195 and 4,683,202. Reagents and hardware for conducting PCR are commercially available. Primers useful for amplifying sequences from the HBM region are preferably complementary to, and hybridize specifically to sequences in the HBM region or in regions that flank a target region therein. HBM sequences generated by amplification may be sequenced directly. Alternatively, the amplified sequence(s) may be cloned prior to sequence analysis.

“Antibodies” may refer to polyclonal and/or monoclonal antibodies and fragments thereof, and immunologic binding equivalents thereof, that can bind to the HBM proteins and fragments thereof or to nucleic acid sequences from the HBM region, particularly from the HBM locus or a portion thereof. The term antibody is used both to refer to a homogeneous molecular entity, or a mixture such as a serum product made up of a plurality of different molecular entities. Proteins may be prepared synthetically in a protein synthesizer and coupled to a carrier molecule and injected over several months into rabbits. Rabbit sera is tested for immunoreactivity to the HBM protein or fragment. Monoclonal antibodies may be made by injecting mice with the proteins, or fragments thereof. Monoclonal antibodies will be screened by ELISA and tested for specific immunoreactivity with IBM protein or fragments thereof Harlow et al, Antibodies: A Laboratory Manual, Cold Spring Harbor Laboratory, Cold Spring Harbor, N.Y. (1988). These antibodies will be useful in assays as well as pharmaceuticals.

“HBM” refers to high bone mass.

“HBM protein” refers to a protein that is identical to a Zmax1 protein except that it contains an alteration of glycine 171 to valine. An HBM protein is defined for any organism that encodes a Zmax1 true homologue. For example, a mouse HBM protein refers to the mouse Zmax1 protein having the glycine 170 to valine substitution.

“HBM gene” refers to the genomic DNA sequence found in individuals showing the HBM characteristic or phenotype, where the sequence encodes the protein indicated by SEQ ID NO: 4. The HBM gene and the Zmax1 gene are allelic. The protein encoded by the HBM gene has the property of causing elevated bone mass, while the protein encoded by the Zmax1 gene does not. The HBM gene and the Zmax1 gene differ in that the HBM gene has a thymine at position 582, while the Zmax1 gene has a guanine at position 582. The HBM gene comprises the nucleic acid sequence shown as SEQ ID NO: 2. The HBM gene may also be referred to as an “HBM polymorphism.”

“Normal,” “wild-type,” “unaffected” and “Zmax1” all refer to the genomic DNA sequence that encodes the protein indicated by SEQ ID NO: 3. The Zmax1 gene has a guanine at position 582. The Zmax1 gene comprises the nucleic acid sequence shown as SEQ ID NO: 1. “Normal,” “wild-type,” “unaffected” and “Zmax1” also refer to allelic variants of the genomic sequence that encodes proteins that do not contribute to elevated bone mass. The Zmax1 gene is common in the human population, while the HBM gene is rare.

“5YWT+EGF” refers to a repeat unit found in the Zmax1 protein, consisting of five YWT repeats followed by an EGF repeat.

“Bone development” generally refers to any process involved in the change of bone over time, including, for example, normal development, changes that occur during disease states, and changes that occur during aging. “Bone development disorder” particularly refers to any disorders in bone development including, for example, changes that occur during disease states and changes that occur during aging. Bone development may be progressive or cyclical in nature. Aspects of bone that may change during development include, for example, mineralization, formation of specific anatomical features, and relative or absolute numbers of various cell types.

“Bone modulation” or “modulation of bone formation” refers to the ability to affect any of the physiological processes involved in bone remodeling, as will be appreciated by one skilled in the art, including, for example, bone resorption and appositional bone growth, by, inter alia, osteoclastic and osteoblastic activity, and may comprise some or all of bone formation and development as used herein.

“Normal bone density” refers to a bone density within two standard deviations of a Z score of 0.

A “Zmax1 system” refers to a purified protein, cell extract, cell, animal, human or any other composition of matter in which Zmax1 is present in a normal or mutant form.

A “surrogate marker” refers to a diagnostic indication, symptom, sign or other feature that can be observed in a cell, tissue, human or animal that is correlated with the HBM gene or elevated bone mass or, both, but that is easier to measure than bone density. The general concept of a surrogate marker is well accepted in diagnostic medicine.

The present invention encompasses the Zmax1 gene and Zmax1 protein in the forms indicated by SEQ ID NOS: 1 and 3, respectively, and other closely related variants, as well as the adjacent chromosomal regions of Zmax1 necessary for its accurate expression. In a preferred embodiment, the present invention is directed to at least 15 contiguous nucleotides of the nucleic acid sequence of SEQ ID NO: 1.

The present invention also encompasses the HBM gene and HBM protein in the forms indicated by SEQ ID NO: 2 and 4, respectively, and other closely related variants, as well as the adjacent chromosomal regions of the HBM gene necessary for its accurate expression. In a preferred embodiment, the present invention is directed to at least 15 contiguous nucleotides of the nucleic acid sequence of SEQ ID NO: 2. More preferably, the present invention is directed to at least 15 contiguous nucleotides of the nucleic acid sequence of SEQ ID NO: 2, wherein one of the 15 contiguous nucleotides is the thymine at nucleotide 582.

The invention also relates to the nucleotide sequence of the Zmax1 gene region, as well as the nucleotide sequence of the HBM gene region. More particularly, a preferred embodiment are the BAC clones containing segments of the Zmax1 gene region B200E21-H and B527D 12-H. A preferred embodiment is the nucleotide sequence of the BAC clones consisting of SEQ ID NOS: 5-11.

The invention also concerns the use of the nucleotide sequence to identify DNA probes for the Zmax1 gene and the HBM gene, PCR primers to amplify the Zmax1 gene and the HBM gene, nucleotide polymorphisms in the Zmax1 gene and the HBM gene, and regulatory elements of the Zmax1 gene and the HBM gene.

This invention describes the further localization of the chromosomal location of the Zmax1 gene and HBM gene on chromosome 11q13.3 between genetic markers D11 S987 and SNP_CONTIG033-6, as well as the DNA sequences of the Zmax1 gene and the HBM gene. The chromosomal location was refined by the addition of more genetic markers to the mapping panel used to map the gene, and by the extension of the pedigree to include more individuals. The pedigree extension was critical because the new individuals that have been genotyped harbor critical recombination events that narrow the region. To identify genes in the region on 11q13.3, a set of BAC clones containing this chromosomal region was identified. The BAC clones served as a template for genomic DNA sequencing, and also as a reagent for identifying coding sequences by direct cDNA selection. Genomic sequencing and direct cDNA selection were used to characterize more than 1.5 million base pairs of DNA from 11q13.3. The Zmax1 gene was identified within this region and the HBM gene was then discovered after mutational analysis of affected and unaffected individuals.

When a gene has been genetically localized to a specific chromosomal region, the genes in this region can be characterized at the molecular level by a series of steps that include: cloning of the entire region of DNA in a set of overlapping clones (physical mapping), characterization of genes encoded by these clones by a combination of direct cDNA selection, exon trapping and DNA sequencing (gene identification), and identification of mutations in these genes by comparative DNA sequencing of affected and unaffected members of the HBM kindred (mutation analysis).

Physical mapping is accomplished by screening libraries of human DNA cloned in vectors that are propagated in E. coli or S. cereviseae using PCR assays designed to amplify unique molecular landmarks in the chromosomal region of interest. To generate a physical map of the HBM candidate region, a library of human DNA cloned in Bacterial Artificial Chromosomes (BACs) was screened with a set of Sequence Tagged Site (STS) markers that had been previously mapped to chromosome 11q12-q13 by the efforts of the Human Genome Project.

STSs are unique molecular landmarks in the human genome that can be assayed by PCR. Through the combined efforts of the Human Genome Project, the location of thousands of STSs on the twenty-two autosomes and two sex chromosomes has been determined. For a positional cloning effort, the physical map is tied to the genetic map because the markers used for genetic mapping can also be used as STSs for physical mapping. By screening a BAC library with a combination of STSs derived from genetic markers, genes, and random DNA fragments, a physical map comprised of overlapping clones representing all of the DNA in a chromosomal region of interest can be assembled.

BACs are cloning vectors for large (80 kilobase to 200 kilobase) segments of human or other DNA that are propagated in E. coli. To construct a physical map using BACs, a library of BAC clones is screened so that individual clones harboring the DNA sequence corresponding to a given STS or set of STSs are identified. Throughout most of the human genome, the STS markers are spaced approximately 20 to 50 kilobases apart, so that an individual BAC clone typically contains at least two STS markers. In addition, the BAC libraries that were screened contain enough cloned DNA to cover the human genome six times over. Therefore, an individual STS typically identifies more than one BAC clone. By screening a six-fold coverage BAC library with a series of STS markers spaced approximately 50 kilobases apart, a physical map consisting of a series of overlapping BAC clones, i.e. BAC contigs, can be assembled for any region of the human genome. This map is closely tied to the genetic map because many of the STS markers used to prepare the physical map are also genetic markers.

When constructing a physical map, it often happens that there are gaps in the STS map of the genome that result in the inability to identify BAC clones that are overlapping in a given location. Typically, the physical map is first constructed from a set of STSs that have been identified through the publicly available literature and World Wide Web resources. The initial map consists of several separate BAC contigs that are separated-by gaps of unknown molecular distance. To identify BAC clones that fill these gaps, it is necessary to develop new STS markers from the ends of the clones on either side of the gap. This is done by sequencing the terminal 200 to 300 base pairs of the BACs flanking the gap, and developing a PCR assay to amplify a sequence of 100 or more base pairs. If the terminal sequences are demonstrated to be unique within the human genome, then the new STS can be used to screen the BAC library to identify additional BACs that contain the DNA from the gap in the physical map. To assemble a BAC contig that covers a region the size of the HBM candidate region (2,000,000 or more base pairs), it is often necessary to develop new STS markers from the ends of several clones.

After building a BAC contig, this set of overlapping clones serves as a template for identifying the genes encoded in the chromosomal region. Gene identification can be accomplished by many methods. Three methods are commonly used: (1) a set of BACs selected from the BAC contig to represent the entire chromosomal region can be sequenced, and computational methods can be used to identify all of the genes, (2) the BACs from the BAC contig can be used as a reagent to clone cDNAs corresponding to the genes encoded in the region by a method termed direct cDNA selection, or (3) the BACs from the BAC contig can be used to identify coding sequences by selecting for specific DNA sequence motifs in a procedure called exon trapping. The present invention includes genes identified by the first two methods.

To sequence the entire BAC contig representing the HBM candidate region, a set of BACs was chosen for subcloning into plasmid vectors and subsequent DNA sequencing of these subclones. Since the DNA cloned in the BACs represents genomic DNA, this sequencing is referred to as genomic sequencing to distinguish it from cDNA sequencing. To initiate the genomic sequencing for a chromosomal region of interest, several non-overlapping BAC clones are chosen. DNA for each BAC clone is prepared, and the clones are sheared into random small fragments which are subsequently cloned into standard plasmid vectors such as pUC18. The plasmid clones are then grown to propagate the smaller fragments, and these are the templates for sequencing. To ensure adequate coverage and sequence quality for the BAC DNA sequence, sufficient plasmid clones are sequenced to yield six-fold coverage of the BAC clone. For example, if the BAC is 100 kilobases long, then phagemids are sequenced to yield 600 kilobases of sequence. Since the BAC DNA was randomly sheared prior to cloning in the phagemid vector, the 600 kilobases of raw DNA sequence can be assembled by computational methods into overlapping DNA sequences termed sequence contigs. For the purposes of initial gene identification by computational methods, six-fold coverage of each BAC is sufficient to yield ten to twenty sequence contigs of 1000 base pairs to 20,000 base pairs.

The sequencing strategy employed in this invention was to initially sequence “seed” BACs from the BAC contig in the HBM candidate region. The sequence of the “seed” BACs was then used to identify minimally overlapping BACs from the contig, and these were subsequently sequenced. In this manner, the entire candidate region was sequenced, with several small sequence gaps left in each BAC. This sequence served as the template for computational gene identification. One method for computational gene identification is to compare the sequence of BAC contig to publicly available databases of cDNA and genomic sequences, e.g. unigene, dbEST, genbank. These comparisons are typically done using the BLAST family of computer algorithms and programs (Altschul et al, J. Mol. Biol., 215:403-410 (1990)). The BAC sequence can also be translated into protein sequence, and the protein sequence can be used to search publicly available protein databases, using a version of BLAST designed to analyze protein sequences (Altschul et al, Nucl. Acids Res., 25:3389-3402 (1997)). Another method is to use computer algorithms such as MZEF (Zhang, Proc. Natl. Acad. Sci., 94:565-568 (1997)) and GRAIL (Uberbacher et al, Methods Enzymol., 266:259-281 (1996)), which predict the location of exons in the sequence based on the presence of specific DNA sequence motifs that are common to all exons, as well as the presence of codon usage typical of human protein encoding sequences.

In addition to identifying genes by computational methods, genes were also identified by direct cDNA selection (Del Mastro et al, Genome Res. 5(2):185-194 (1995)). In direct cDNA selection, cDNA pools from tissues of interest are prepared, and the BACs from the candidate region are used in a liquid hybridization assay to capture the cDNAs which base pair to coding regions in the BAC. In the methods described herein, the cDNA pools were created from several different tissues by random priming the first strand cDNA from polyA RNA, synthesizing the second strand cDNA by standard methods, and adding linkers to the ends of the cDNA fragments. The linkers are used to amplify the cDNA pools. The BAC clones are used as a template for in vitro DNA synthesis to create a biotin labelled copy of the BAC DNA. The biotin labelled copy of the BAC DNA is then denatured and incubated with an excess of the PCR amplified, Tinkered cDNA pools which have also been denatured. The BAC DNA and cDNA are allowed to anneal in solution, and heteroduplexes between the BAC and the cDNA are isolated using streptavidin coated magnetic beads. The cDNAs that are captured by the BAC are then amplified using primers complimentary to the linker sequences, and the hybridization/selection process is repeated for a second round. After two rounds of direct cDNA selection, the cDNA-fragments are cloned, and a library of these direct selected fragments is created.

The cDNA clones isolated by direct selection are analyzed by two methods. Since a pool of BACs from the HBM candidate region is used to provide the genomic DNA sequence, the cDNAs must be mapped to individual BACs. This is accomplished by arraying the BACs in microtiter dishes, and replicating their DNA in high density grids. Individual cDNA clones are then hybridized to the grid to confirm that they have sequence identity to an individual BAC from the set used for direct selection, and to determine the specific identity of that BAC. cDNA clones that are confirmed to correspond to individual BACs are sequenced. To determine whether the cDNA clones isolated by direct selection share sequence identity or similarity to previously identified genes, the DNA and protein coding sequences are compared to publicly available databases using the BLAST family of programs.

The combination of genomic DNA sequence and cDNA sequence provided by BAC sequencing and by direct cDNA selection yields an initial list of putative genes in the region. The genes in the region were all candidates for the HBM locus. To further characterize each gene, Northern blots were performed to determine the size of the transcript corresponding to each gene, and to determine which putative exons were transcribed together to make an individual gene. For Northern blot analysis of each gene, probes were prepared from direct selected cDNA clones or by PCR amplifying specific fragments from genomic DNA or from the BAC encoding the putative gene of interest. The Northern blots gave information on the size of the transcript and the tissues in which it was expressed. For transcripts which were not highly expressed, it was sometimes necessary to perform a reverse transcription PCR assay using RNA from the tissues of interest as a template for the reaction.

Gene identification by computational methods and by direct cDNA selection provides unique information about the genes in a region of a chromosome. When genes are identified, then it is possible to examine different individuals for mutations in each gene.

I. Phenotyping Using DXA Measurements

Spinal bone mineral content (BMC) and bone mineral density (BMD) measurements performed at Creighton University (Omaha, Nebr.) were made by DXA using a Norland Instruments densitometer (Norland XR2600 Densitometer, Dual Energy X-ray Absorptiometry, DXA). Spinal BMC and BMD at other locations used the machinery available. There are estimated to be 800 DXA machines currently operating in the U.S. Most larger cities have offices or imaging centers which have DXA capabilities, usually a Lunar or Hologic machine. Each location that provided spine BMC and BMD data included copies of the printouts from their machines to provide verification that the regions of interest for measurement of BMD have been chosen appropriately. Complete clinical histories and skeletal radiographs were obtained.

The HBM phenotype is defined by the following criteria: very high spinal BMD; a clinical history devoid of any known high bone mass syndrome; and skeletal radiographs showing a normal shape of the appendicular skeleton.

II. Genotyping of Microsatellite Markers

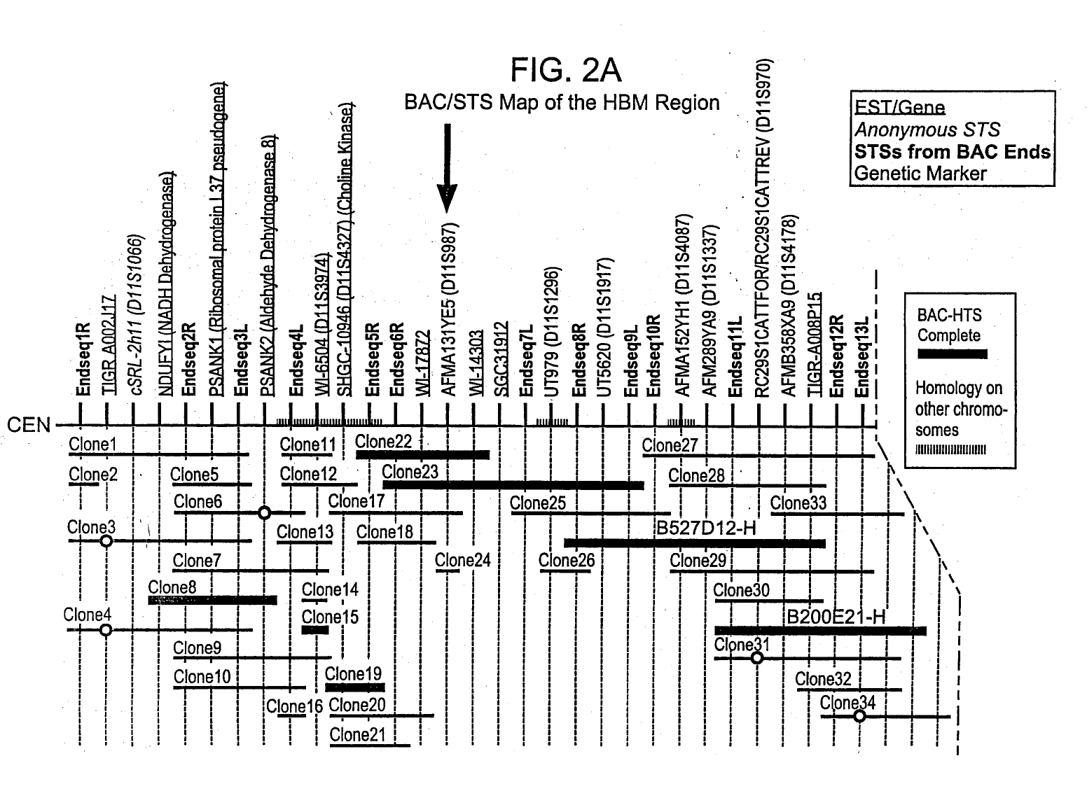

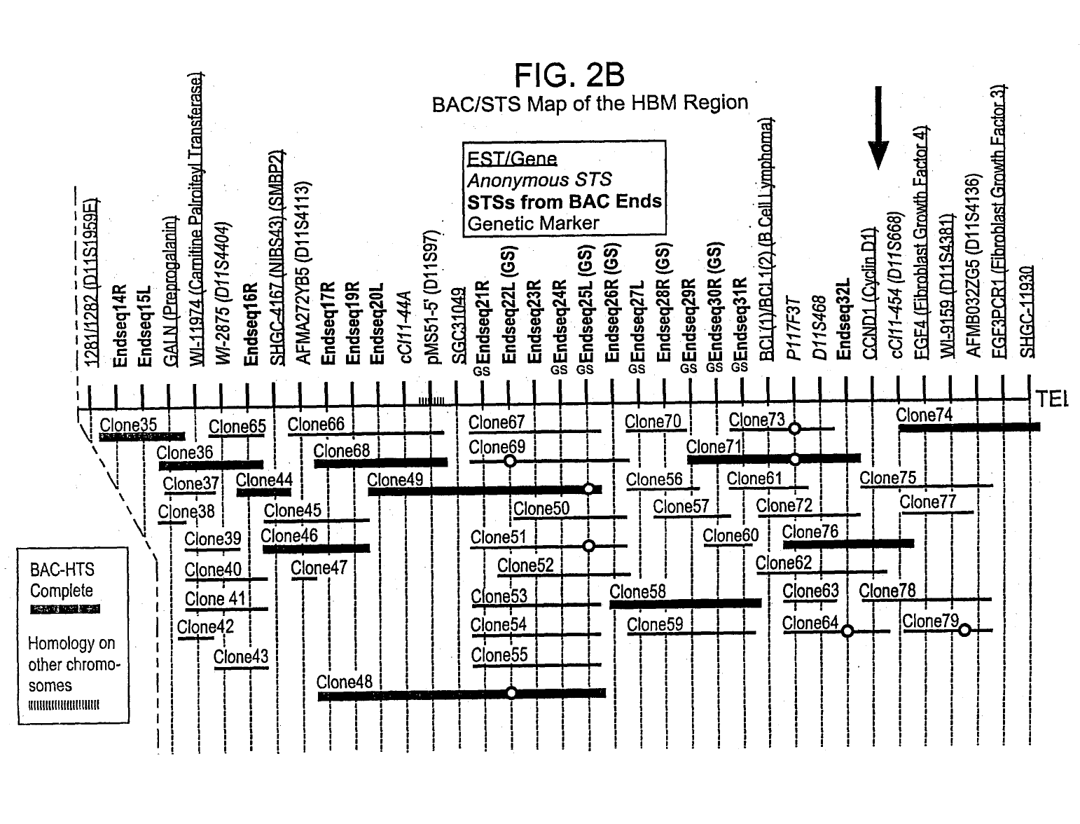

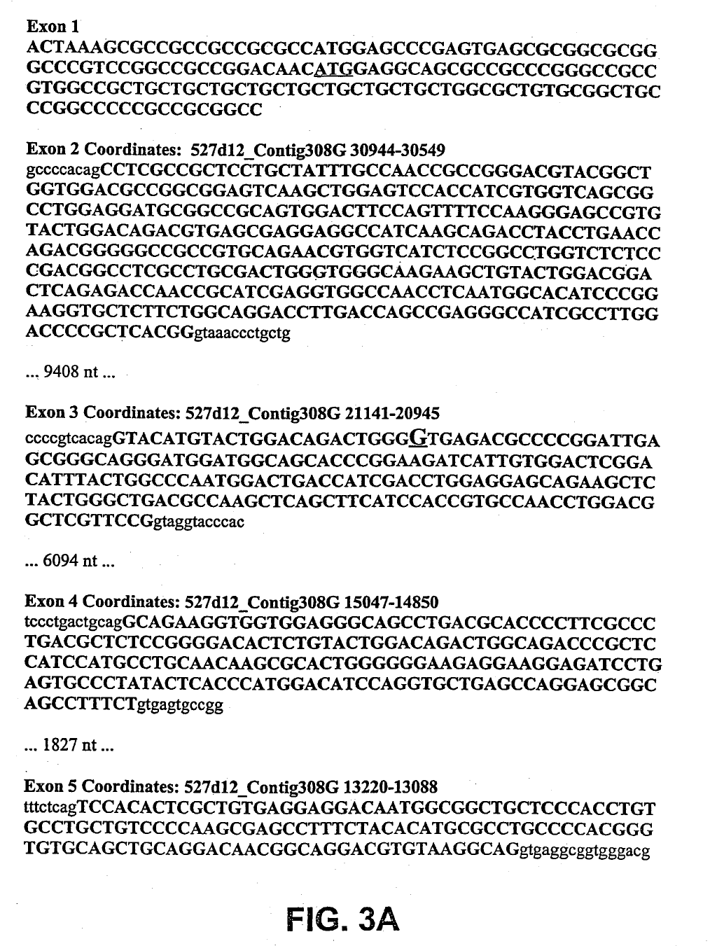

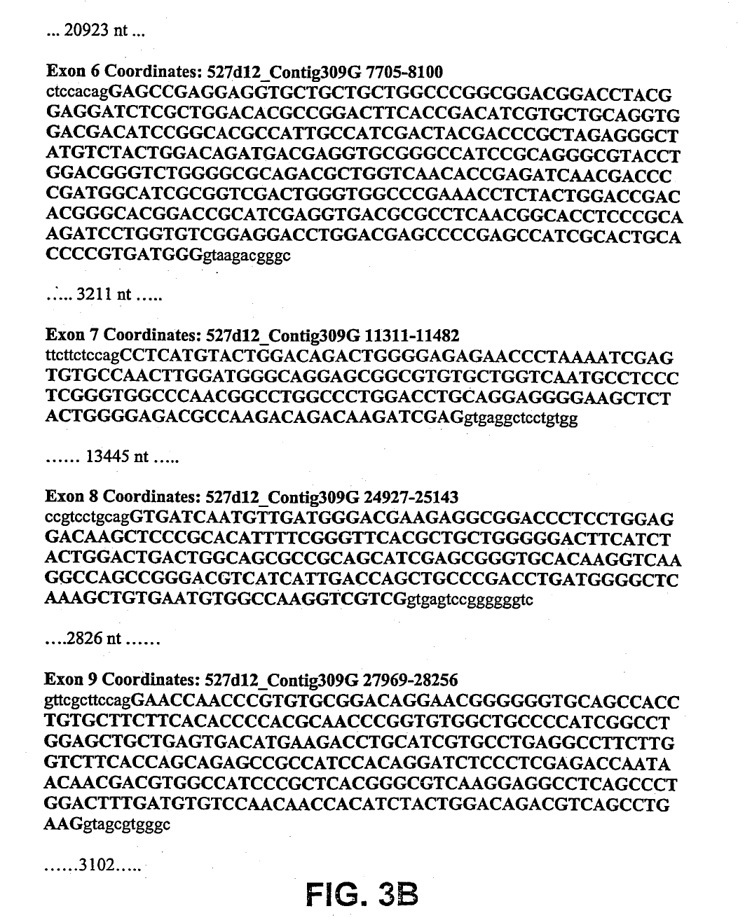

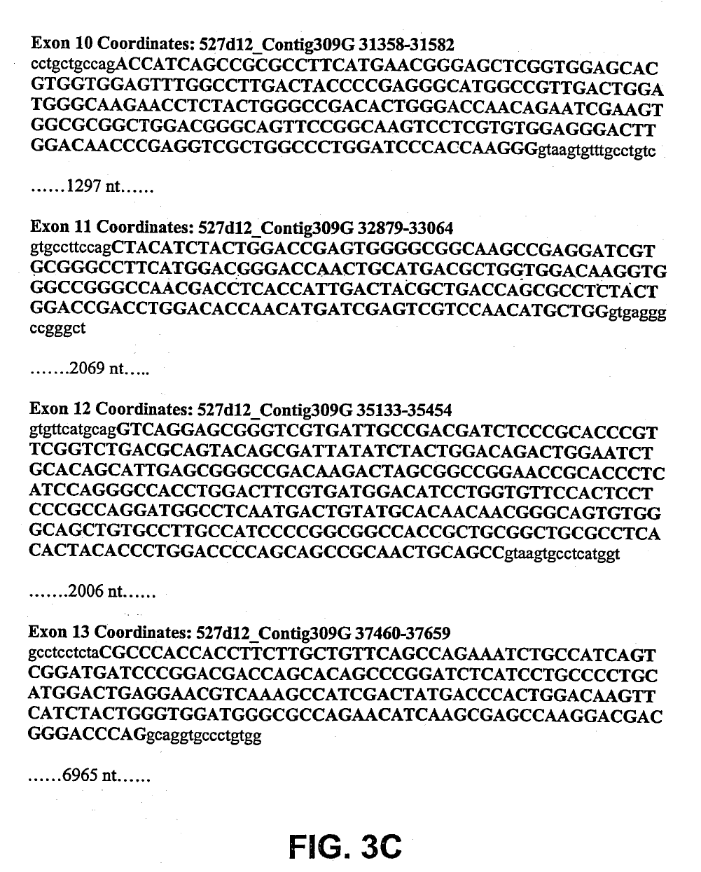

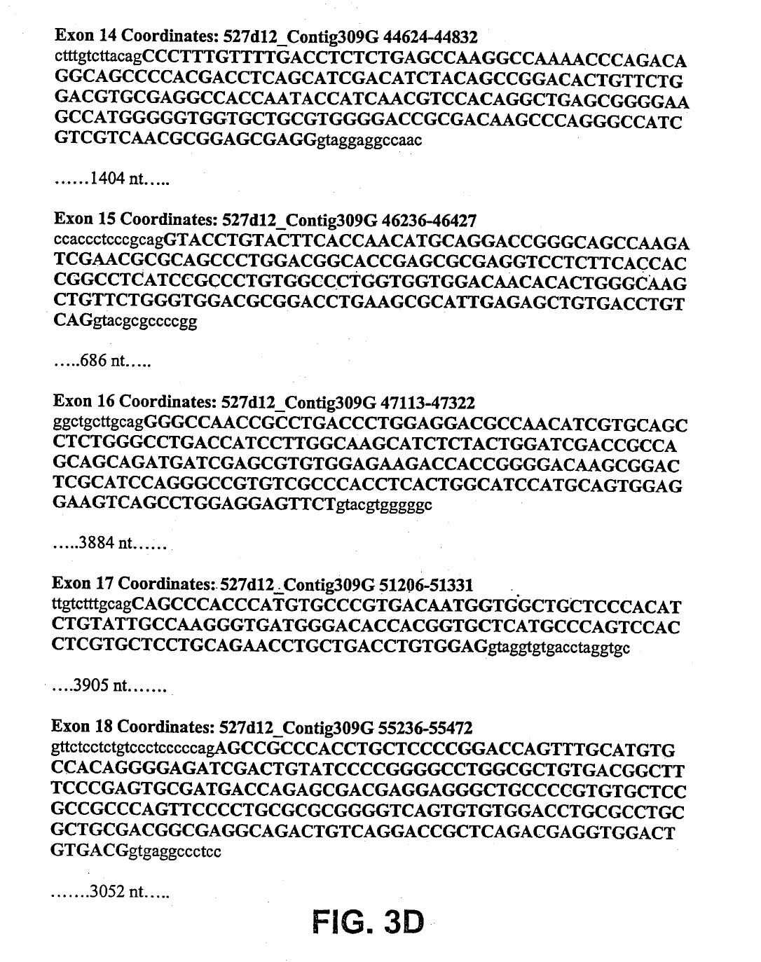

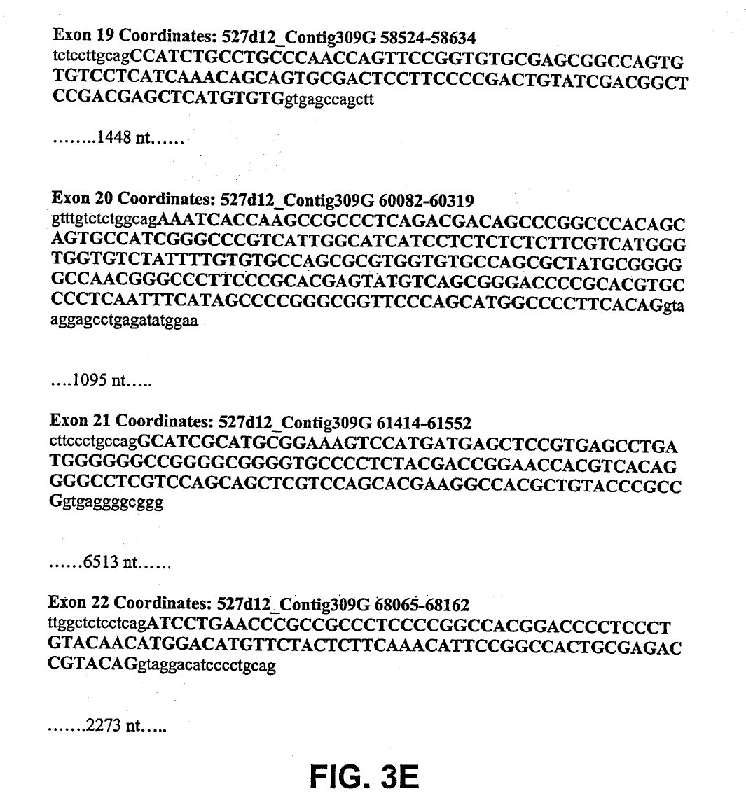

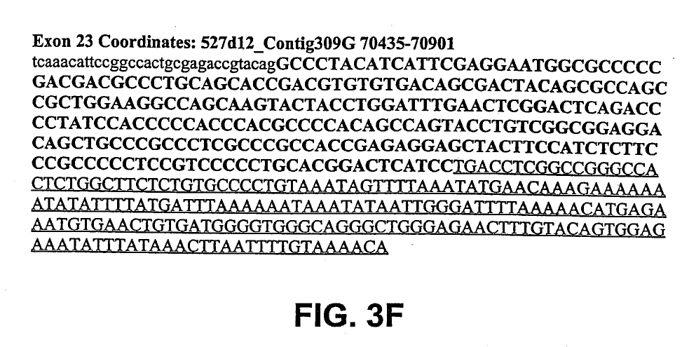

To narrow the genetic interval to a region smaller than that originally reported by Johnson et al, Am. J. Hum. Genet., 60:1326-1332 (1997), additional microsatellite markers on chromosome 11q12-13 were typed. The new markers included: D11S4191, D11S1883, D11S1785, D11S4113, D11S4136, D11S4139, (Dib, et al, Nature, 380:152-154 (1996), FGF3 (Polymeropolous, et al, Nucl. Acid Res., 18:7468 (1990)), as well as GTC_HBM_Marker_{—}1, GTC_HBM_Marker_{—}2, GTC_HBM_Marker_{—}3, GTC_HBM_Marker_{—}4, GTC_HBM_Marker_{—}5, GTC_HBM_Marker_{—}6, and GTC_HBM_Marker_{—}7 (See FIG. 2).

Blood (20 ml) was drawn into lavender cap (EDTA containing) tubes by a certified phlebotomist. The blood was stored refrigerated until DNA extraction. DNA has been extracted from blood stored for up to 7 days in the refrigerator without reduction in the quality or quantity of yield. For those subjects that have blood drawn at distant sites, a shipping protocol was successfully used on more than a dozen occasions. Blood samples were shipped by overnight express in a styrofoam container with freezer packs to provide cooling. Lavender cap tubes were placed on individual plastic shipping tubes and then into “zip-lock” biohazard bags. When the samples arrived the next day, they were immediately processed to extract DNA.

The DNA extraction procedure used a kit purchased from Gentra Systems, Inc. (Minneapolis, Minn.). Briefly, the procedure involved adding 3 volumes of a red blood cell lysis buffer to the whole blood. After incubations for 10 minutes at room temperature, the solution was centrifuged in a Beckman tabletop centrifuge at 2,000×g for 10 minutes. The white blood cell pellet was resuspended in Cell Lysis Buffer. Once the pellet was completely resuspended and free of cell clumps, the solution was digested with RNase A for 15 minutes at 37° C. Proteins were precipitated by addition of the provided Protein Precipitation Solution and removed by centriftigation. The DNA was precipitated out of the supernatant by addition of isopropanol. This method was simple and fast, requiring only 1-2 hours, and allowed for the processing of dozens of samples simultaneously. The yield of DNA was routinely >8 mg for a 20 ml sample of whole blood and had a MW of >50 kb. DNA was archived by storing coded 50 μg aliquots at −80° C. as an ethanol precipitate.

DNA was genotyped using one fluorescently labeled oligonucleotide primer and one unlabeled oligonucleotide primer. Labeled and unlabeled oligonucleotides were obtained from Integrated DNA Technologies, Inc. (Coralville, Iowa). All other reagents for microsatellite genotyping were purchased from Perkin Elmer-Applied Biosystems, Inc. (“PE-ABI”) (Norwalk, Conn.). Individual PCR reactions were performed for each marker, as described by PE-ABI using AmpliTag DNA Polymerase. The reactions were added to 3.5 μl of loading buffer containing deionized formamide, blue dextran and TAMRA 350 size standards (PE-ABI). After heating at 95° C. for 5 minutes to denature the DNA, the samples were loaded and electrophoresed as described in the operator's manual for the Model 377 DNA Sequencer (PE-ABI, Foster City, Calif.). After gel electrophoresis, the data was analyzed using PE-ABI GENESCAN™ and GENOTYPER™ software. First, within the GENESCAN™ software, the lane tracking was manually optimized prior to the first step of analysis. After the gel lane data was extracted, the standard curve profiles of each lane were examined and verified for linearity and size calling. Lanes, which had problems with either of these parameters, were re-tracked and verified. Once all lanes were tracked and the size standards were correctly identified, the data were imported into GENOTYPER™ for allele identification. To expedite allele calling (binning), the program Linkage Designer from the Internet web-site of Dr. Guy Van Camp (http://alt.www.uia.ac.be/u/dnalab/ld.html) was used. This program greatly facilitates the importing of data generated by GENOTYPER™ into the pedigree drawing program Cyrillic (Version 2.0, Cherwell Scientific Publishing Limited, Oxford, Great Britain) and subsequent linkage analysis using the program LINKAGE (Lathrop et al, Am. J. Hum. Genet., 37:482-498 (1985)).

III. Linkage Analysis

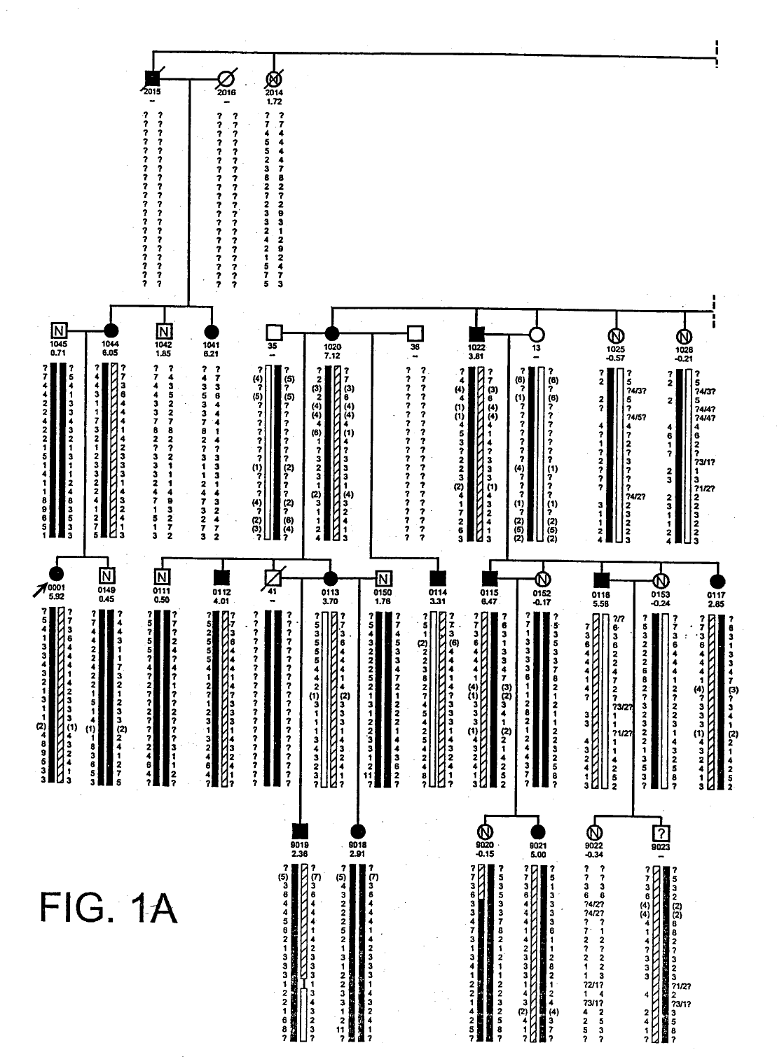

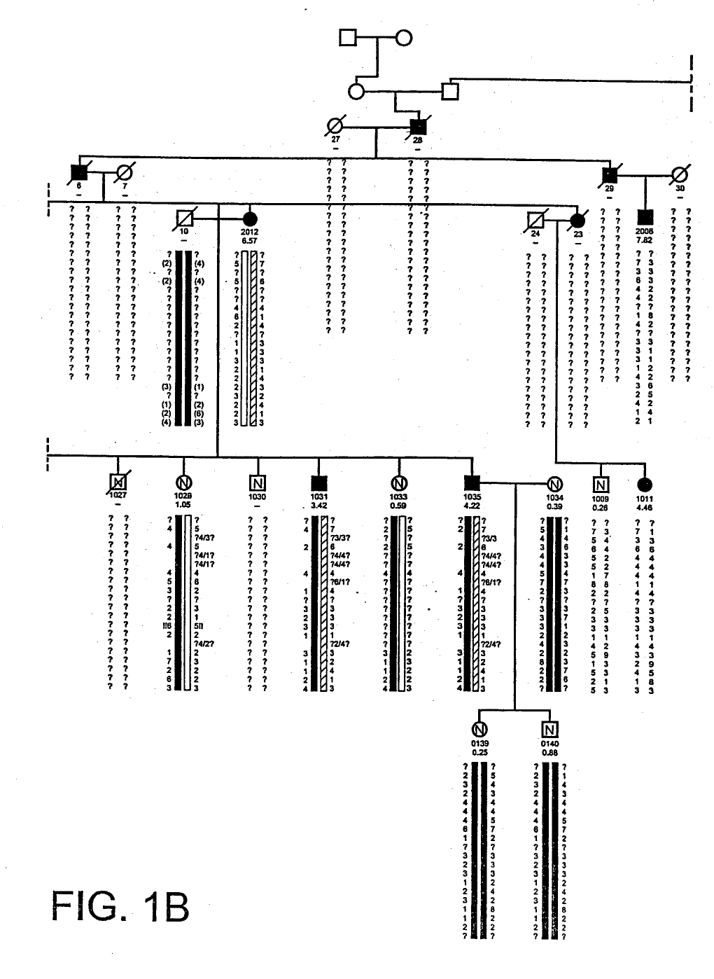

FIG. 1 demonstrates the pedigree of the individuals used in the genetic linkage studies for this invention. Specifically, two-point linkage analysis was performed using the MLINK and LIMAP components of the program LINKAGE (Lathrop et al, Am. J. Hum. Genet., 37:482-498 (1985)). Pedigree/marker data was exported from Cyrillic as a pre-file into the Makeped program and converted into a suitable ped-file for linkage analysis.

The original linkage analysis was performed using three models: (i) an autosomal dominant, fully penetrant model, (ii) an autosomal dominant model with reduced penetrance, and (iii) a quantitative trait model. The HBM locus was mapped to chromosome 11q12-13 by analyzing DNA for linked markers from 22 members of a large, extended kindred. A highly automated technology was used with a panel of 345 fluorescent markers which spanned the 22 autosomes at a spacing interval ranging from 6-22 cM. Only markers from this region of chromosome 11 showed evidence of linkage (LOD score˜3.0). The highest LOD score (5.74) obtained by two-point and multipoint analysis was D11S987 (map position 55 in FIG. 2). The 95% confidence interval placed the HBM locus between markers D11S905 and D11S937 (map position 41-71 in FIG. 2). Haplotype analysis also places the Zmax1 gene in this same region. Further descriptions of the markers D11S987, D11S905, and D11S937 can be found in Gyapay et al, Nature Genetics, Vol. 7, (1994).

In this invention, the inventors report the narrowing of the HBM interval to the region between markers D11S987 and GTC_HBM_Marker_{—}5. These two markers lie between the delimiting markers from the original analysis (D11S11S905 and D11S937) and are approximately 3 cM from one another. The narrowing of the interval was accomplished using genotypic data from the markers D11S4191, D11S1883, D11S1785, D11S4113, D11S4136, D11S4139,(Dib et al, Nature, 380:152-154 (1996)), FGF3 (Polymeropolous et al, Nucl. Acid Res., 18:7468 (1990)) (information about the genetic markers can be found at the internet site of the Genome Database, http://gdbwww.gdb.org/), as well as the markers GTC_HBM_Marker_{—}1, GTC_HBM_Marker_{—}2, GTC_HBM_Marker_{—}3, GTC_HBM_Marker_{—}4, GTC_HBM_Marker_{—}5, GTC_HBM_Marker_{—}6, and GTC_HBM_Marker_{—}7.

As shown in FIG. 1, haplotype analysis with the above genetic markers identifies recombination events (crossovers) in individuals 9019 and 9020 that significantly refine the interval of chromosome 11 to which the Zmax1 gene is localized. Individual 9019 is an HBM-affected individual that inherits a portion of chromosome 11 from the maternal chromosome with the HBM gene, and a portion from the chromosome 11 homologue. The portion inherited from the HBM gene-carrying chromosome includes markers D11S935, D11S1313, GTC_HBM_Marker_{—}4, D11S987, D11S1296, GTC_HBM_Marker_{—}6, GTC_HBM_Marker_{—}2, D11S970, GTC_HBM_Marker_{—}3, D11S4113, GTC_HBM_Marker_{—}1, GTC_HBM_Marker_{—}7 and GTC_HBM_Marker_{—}5. The portion from D11S4136 and continuing in the telomeric direction is derived from the non-HBM chromosome. This data places the Zmax1 gene in a location centromeric to the marker GTC_HBM_Marker_{—}5. Individual 9020 is an unaffected individual who also exhibits a critical recombination event. This individual inherits a recombinant paternal chromosome 11 that includes markers D11S935, D11S1313, GTC_HBM_Marker_{—}4, D11S987, D11S1296 and GTC_HBM_Marker_{—}6 from her father's (individual 0115) chromosome 11 homologue that carries the HBM gene, and markers GTC_HBM_Marker_{—}2, D11S970, GTC_HBM_Marker_{—}3, GTC_HBM_Marker_{—}1, GTC_HBM_Marker_{—}7, GTC_HBM_Marker_{—}5, D11S4136, D11S4139, D11S1314, and D11S937 from her father's chromosome 11 that does not carry the HBM gene. Marker D11S4113 is uninformative due to its homozygous nature in individual 0115. This recombination event places the centromeric boundary of the HBM region between markers D11S1296 and D11S987.

Two-point linkage analysis was also used to confirm the location of the Zmax1 gene on chromosome 11. The linkage results for two point linkage analysis under a model of full penetrance are presented in Table 1 below. This table lists the genetic markers in the first column and the recombination fractions across the top of the table. Each cell of the column shows the LOD score for an individual marker tested for linkage to the Zmax1 gene at the recombination fraction shown in the first row. For example, the peak LOD score of 7.66 occurs at marker D11S970, which is within the interval defined by haplotype analysis.

TABLE 1

| Marker | 0.0 | 0.05 | 0.1 | 0.15 | 0.2 | 0.25 | 0.3 | 0.35 | 0.4 |
|---|---|---|---|---|---|---|---|---|---|
| D11S935 | −infinity | 0.39 | 0.49 | 0.47 | 0.41 | 0.33 | 0.25 | 0.17 | 0.10 |
| D11S1313 | −infinity | 2.64 | 2.86 | 2.80 | 2.59 | 2.30 | 1.93 | 1.49 | 1.00 |
| D11S987 | −infinity | 5.49 | 5.18 | 4.70 | 4.13 | 3.49 | 2.79 | 2.03 | 1.26 |
| D11S4113 | 4.35 | 3.99 | 3.62 | 3.24 | 2.83 | 2.40 | 1.94 | 1.46 | 0.97 |
| D11S1337 | 2.29 | 2.06 | 1.81 | 1.55 | 1.27 | 0.99 | 0.70 | 0.42 | 0.18 |
| D11S970 | 7.66 | 6.99 | 6.29 | 5.56 | 4.79 | 3.99 | 3.15 | 2.30 | 1.44 |
| D11S4136 | 6.34 | 5.79 | 5.22 | 4.61 | 3.98 | 3.30 | 2.59 | 1.85 | 1.11 |
| D11S4139 | 6.80 | 6.28 | 5.73 | 5.13 | 4.50 | 3.84 | 3.13 | 2.38 | 1.59 |
| FGF3 | 0.59 | 3.23 | 3.15 | 2.91 | 2.61 | 2.25 | 1.84 | 1.40 | 0.92 |
| D11S1314 | 6.96 | 6.49 | 5.94 | 5.34 | 4.69 | 4.01 | 3.27 | 2.49 | 1.67 |
| D11S937 | −infinity | 4.98 | 4.86 | 4.52 | 4.06 | 3.51 | 2.88 | 2.20 | 1.47 |

A single nucleotide polymorphism (SNP) further defines the HBM region. This SNP is termed SNP_Contig033-6 and is located 25 kb centromeric to the genetic marker GTC_HBM_Marker_{—}5. This SNP is telomeric to the genetic marker GTC_HBM_Marker_{—}7. SNP_Contig033-6 is present in HBM-affected individual 0113. However, the HBM-affected individual 9019, who is the son of 0113, does not carry this SNP. Therefore, this indicates that the crossover is centromeric to this SNP. The primer sequence for the genetic markers GTC_HBM_Marker_{—}5 and GTC_HBM_Marker_{—}7 is shown in Table 2 below.

TABLE 2

| | | |
|---|---|---|
| Marker | Primer (Forward) | Primer (Reverse) |

| | | |
|---|---|---|
| GTC_FBM_Marker_5 | TTTTGGGTACACAATTCAGTCG | AAAACTGTGGGTGCTTCTGG |
| GTC_HBM_Marker_7 | GTGATTGAGCCAATCCTGAGA | TGAGCCAAATAAACCCCTTCT |

The kindred described have several features of great interest, the most important being that their bones, while very dense, have an absolutely normal shape. The outer dimensions of the skeletons of the HBM-affected individuals are normal, and, while medullary cavities are present, there is no interference with hematopoiesis. The HBM-affected members seem to be resistant to fracture, and there are no neurologic symptoms, and no symptoms of impairment of any organ or system function in the members examined. HBM-affected members of the kindred live to advanced age without undue illness or disability. Furthermore, the HBM phenotype matches no other bone disorders such as osteoporosis, osteoporosis pseudoglioma, Engelmann's disease, Ribbing's disease, hyperphosphatasemia, Van Buchem's disease, melorheostosis, osteopetrosis, pycnodysostosis, sclerostenosis, osteopoikilosis, acromegaly, Paget's disease, fibrous dysplasia, tubular stenosis, osteogenesis imperfecta, hypoparathyroidism, pseudohypoparathyroidism, pseudopseudohypoparathyroidism, primary and secondary hyperparathyroidism and associated syndromes, hypercalciuria, medullary carcinoma of the thyroid gland, osteomalacia and other diseases. Clearly, the HBM locus in this family has a very powerful and substantial role in regulating bone density, and its identification is an important step in understanding the pathway(s) that regulate bone density and the pathogenesis of diseases such as osteoporosis.

In addition, older individuals carrying the HBM gene, and therefore expression of the HBM protein, do not show loss of bone mass characteristic of normal individuals. In other words, the HBM gene is a suppressor of osteoporosis. In essence, individuals carrying the HBM gene are dosed with the HBM protein, and, as a result, do not develop osteoporosis. This in vivo observation is strong evidence that treatment of normal individuals with the HBM gene or protein, or a fragment thereof, will ameliorate osteoporosis.

IV. Physical Mapping

To provide reagents for the cloning and characterization of the HBM locus, the genetic mapping data described above were used to construct a physical map of the region containing Zmax1 on chromosome 11q13.3. The physical map consists of an ordered set of molecular landmarks, and a set of BAC clones that contain the Zmax1 gene region from chromosome 11q13.3.

Various publicly available mapping resources were utilized to identify existing STS markers (Olson et al, Science, 245:1434-1435 (1989)) in the HBM region. Resources included the GDB, the whitehead Institute Genome Center, dbSTS and dbEST (NCBI), 11 db, the University of Texas Southwestern GESTEC, the Stanford Human Genome Center, and several literature references (Courseaux et al, Genomics, 40:13-23 (1997), Courseaux et al, Genomics, 37:354-365 (1996), Guru et al, Genomics, 42:436-445 (1997), Hosoda et al, Genes Cells, 2:345-357 (1997), James et al, Nat. Genet., 8:70-76 (1994), Kitamura et al, DNA Research, 4:281-289 (1997), Lemmens et al, Genomics, 44:94-100 (1997), Smith et al, Genome Res., 7:835-842 (1997)). Maps were integrated manually to identify markers mapping to the region containing Zmax1.

Primers for existing STSs were obtained from the GDB or literature references are listed in Table 3 below. Thus, Table 3 shows the STS markers used to prepare the physical map of the Zmax1 gene region.

TABLE 3

| HBM STS Table | | | | | | | |
|---|---|---|---|---|---|---|---|
| STS Name | Locus Nam | Type | GDB Access. | Size (kb | Forward Primer | Reverse Primer | Gene Name |
| ACTN3 | | Gene | GDB:197568 | 0.164 | CTGGACTACGTGGCCTTCTC | TTCAGAAGCACTTGGCTGG | Aclinlin, alpha 3-skeletal muscle |
| PC-B/PC-Y | | Gene | GDB:197884 | 0.125 | CTCAGTGCCATGAAGATGGA | CAAGATCACTCGATCTCCAGG | Pyruvate Carboxylase |
| | D11S2181 | Gene | | 0.322 | GTTTCAGGAGACTCAGAGTC | TTCTGCAGGVTGCTGTTGAG | Adenosine Receptor (A2) Gene |
| ADRBK1 | | Gene | GDB:4590179 | 0.117 | TATTGTGATTTCCCGTGGC | GCCCTCTGTCCTGACTTCAGG | Beta-adrenergic receptor kinase |
| PSANK3 | | GENE | | 0.259 | GAGAAAGAAATAAGGGGACC | TGCTTTGTAAAGCACTGAGA | sim. lo Human andogenous retrovirus |
| | | | | | | | mRNA long terminal repeat |
| PP1(½)/PP1(2/2) | | Gene | GDB:197588 | 0.208 | GAAGTACGGGCAGTTCAGTGGCCT | ATACACCAAGGTCCATGTTCCCCGT | Protein phosphatase 1, catalytic sub- |
| | | | | | | | unit, alpha isoform |
| GSTP1.PCR1 | | Gene | GDB:270066 | 0.19 | AGCCTGGGCCACAGCGTGAGACTAC | TCCCGGAGCTTGCACACCCGCTTCA | Glutathion S-transferaes pl. |
| NDUFV1 | | Gene | | 0.521 | CATGTGCCCACCTCATTCAT | CAAGATTCTGTAGCTTCTGG | NADH dehydrogenase (ubiquinone) flavo- |
| | | | | | | | protein 1 (51 kD) |
| PSANK2 | | GENE | | 0.157 | CAGAGAAGTCAAGGGACTTG | ATCCTCTCACATCCCACACT | Aldahyde Dehydrogenase 8 (ALDH8) |
| PSANK1 | | EST | | 0.3 | CAAGGCTAAAAGACGAAAAA | TCAGGAGCATTTCATCTTTT | Human ribosomal protein L37 (PSANK1) |
| | | | | | | | pseudogene |
| UT5620 | D11S1917 | MSAT | GDB:314521 | 0.211 | AAGTCGAGGCTGCAAGGAG | GCCTGTGTTCCTTTCAGTA | |
| AFM289ya9 | D11S1337 | MSAT | GDB:199805 | 0.287 | AAGGTGTGAGGATCACTGG | AGCTCATGGGGGCTATT | |
| GALN | | Gene | | 0.322 | GCTTCTCCGAGTGTATCAAC | ATGGCAGAGGACTTAGAACA | Preprogalanin (GAL 1) |
| pMS51 | D11697 | VNTR | GDB:177850 | | GATCAGCGAACTTCCTCTCGGCTC | TCCACATTGAGGACTGTGGGAACG | |
| BCL1(1)/BCL1(2) | | Gene | | 0.205 | GCTAATCACAGTCTAACCGA | TTGCACTGTCTTGGATGCA | B-cell CLL/lymphoma 1-Cyclin D1(PRAD1 |
| | | | | | | | gene) |
| CCND1 | | Gene | GDB:4590141 | 0.248 | GCACAGTGTAGTGGGGTTCTAGGC | CAGGCGCAAAGGACATGCACACGGC | Cyclin D1 |
| FGF4 | | Gene | GDB:4590113 | 0.549 | CACCGATGAGTGCACGTTCAAGGAG | CAGACAGAGATGCTCCACGCCATAC | Fibroblast growth factor 4 |
| FGF3 PCR1 | | Gene | GDB:188627 | 0.161 | TTTCTGGGTGTGTCTGAAT | ACACAGTTGCTCTAAAGGGT | Fibroblast growth factor 3 |
| AFM164ZF12 | D116913 | MSAT | GDB:188151 | 0.22 | CATTTGGGAAATCCAGAAGA | TAGGTGTCTTATTTTTTGTTGCTTC | |
| AFMA190YD5 | | MSAT | GDB:122329 | 0.275 | GACATACGATGAACACTATAAGAGG | CAACCATACCAGGGATAAG | |
| SHGC-15295 | D11S4889 | STS | GDB:740600 | 0.147 | GAACAAGAGGGGTAAGTTGGC | TGAGGACACAGATACTGATGGG | |
| SHGC-3084 | D11S4540 | STS | GDB:740102 | 0.167 | GAAGTGTTCCCTCTTAAATTCTTTG | GAACTATATTGTAGTTAGTGAGGAG | |
| SHGC-14407 | D11S4664 | STS | GDB:740516 | 0.158 | CCTGTAACCCCCAGTCCC | TCTTGCTTCCTAAGTTTCTCGG | |
| SHGC-10946 | D11S4327 | Gene | GDB:674522 | 0.311 | ACTCCATCCACCTCATCACTG | TGCTGTTTGCCTCATCTGAC | Choline Kinase |
| S515 | D11S703 | STS | GDB:196290 | 0.166 | GTGGACAGGCATAGCTGAGG | TGTTCACTCTTCTGCCTGCAG | |
| AFM147XD10 | D11S1889 | MSAT | GDB:307895 | 0.183 | AGCTGGACTCTCACAGAATG | CAAGAGGCTGGTAGAAGGTG | |
| AFMA131YE5 | D11S987 | MSAT | GDB:195002 | 0.082 | GACTCCAGTCTGGGCAATAAAAGC | GGTGGCAGCATGACCTCTAAAG | |
| AFMb358xa9 | D11S4178 | MSAT | GDB:611922 | 0.237 | CAGGCCCAGTCTCTTG | CGTGTCCAGATGAAAGTG | |
| AFMa272yb5 | D11S4113 | MSAT | GDB:608115 | 0.218 | ACCTCACGGTGTAATCCC | CTTGAAGCCCATCTTTGC | |
| WI-17803 | | EST | GDB:4581644 | 0.15 | TATTTGCAAAGCTTGAGACTTCT | AATCACTGTGCTTTGTTGCC | |
| SGC31923 | | EST | GDB:4578606 | 0.126 | ACTTTATTGTCAGCGTGGGC | ACTCCCTCGATGGCTTCC | |
| WI-7741 | D11S4384 | GENE | GDB:677852 | 0.324 | GAGCAGGGGAGAGAAGGC | CCCAACTGGCTTGTTTTATTG | Transformation-sensitive protein EF SSP |
| | | | | | | | 3521 |
| SGC35223 | | EST | GDB:4582598 | 0.13 | AGCCACTTTATTGTTATTTTGATGC | AAGAGTGAACAAAAGCAAACATACC | 2NF162-splicing factor 1 |
| WI-16754 | | EST | GDB:4578377 | 0.15 | GTGGAGTGTGGGATTGGG | TACTGTTCTTGATAAGTATGTCGGC | |
| WI-6315 | D11S4418 | EST | GDB:678804 | 0.224 | ATGCTTTTGCATGATTCTAATTATT | TCCCCCAAAAGAATGTAAAGG | |
| WI-16915 | | EST | GDB:4584055 | 0.125 | GTGGTCTTCCTTGTGTGCTG | ATCACCCAGGCCAGGGAT | Milogan inducible gene (MIG-2) |
| SGC30608 | | EST | | 0.128 | TCAGAAGCAGAACTGTTTTTAACA | CCTGCTTGAAGTTCTAGAGCC | |
| WI-17663 | | EST | GDB:4583346 | 0.126 | CAAGCCGGGTTTTATTGAAA | GATGCCAGGACCATGGAC | |
| WI-6383 | | Gene | GDB:1222237 | 0.199 | GCATATAGAAACAATTTATTGCCG | CTCTGAAGCAGGGACCAGAG | Human lat interactive protein (TIP60) |
| SGC31567 | | Gene | GDB:4578432 | 0.207 | CTACCACACCACACCAGGC | CAAGCGAAAGCTGCCTTC | Calcium activated neutral protease |
| | | | | | | | large subunit,muCANP, caipain |
| SGC30658 | | EST | GDB:4584037 | 0.15 | GTTGTCTTGACTTCAGGTCTGTC | TTTTCCTTCAACAATCACTACTCC | |
| SGC34590 | | EST | | 0.13 | GCGTGGGGATATAGAGGTCA | TACGTGGCCAAGAAGCTAGG | |
| SGC33927 | | EST | GDB:4582382 | 0.15 | TAATATATCCCCAGTCTAAGGCAT | AGCTTGCAGATGGAGCCC | |
| WI-8671 | | EST | GDB:1222235 | 0.124 | TGGTTTTAAACCTTTAATGAGAAAA | TGTTGATCTATACCCTGTTTCCG | |
| WI-12334 | | EST | GDB:1222257 | 0.127 | AATTATTTAAAAGAGAGGAAAGGCA | TGGCTGTGAACTTCCTCTGA | |
| WI-18402 | | EST | GDB:4581874 | 0.113 | GGTTACAGAAAAACATTTGAGAGAT | TGAGCTTTAGTTCCCTTCTCTG | |
| WI-18671 | | EST | GDB:4584947 | 0.131 | TTGAAAAACCATTTATTTCACCG | TCTGCGGCTGTTGGATT | Hark |
| WI-12856 | | EST | GDB:4576606 | 0.209 | TTGAAAAACCATTTATTTCACCG | TGTTCTCTTCTCCCAGCAGG | Hark |
| SGC33767 | | EST | GDB:4581106 | 0.15 | CTTTATTGAAAACATTGAGTGCA | TTGTCAAATTCCCCCCAAAA | |
| AFM3436YB5 | | MSAT | GDB:1222332 | 0.181 | AAACCACGACCNCCAA | CCCTGGAAAGGTAAGATGCT | |
| SGC33744 | | EST | GDB:4575826 | 0.15 | CTTTTGGTAGAGACAAGGTCTCA | TATCTGTCTGTAGTGCTTCAAATGT | |
| SGC32272 | | EST | GDB:4581592 | 0.135 | GACGAAGGTGATTCAGGGC | ACTGAAGAACTCTTGTCCT | |
| SGC34148 | | EST | GDB:4583084 | 0.1 | CAGATAAAAGAGTCACTATGGCTCA | CACTTCTCCCACTTTGTCCC | |
| WI-18546 | | EST | GDB:4574598 | 0.133 | TTATTGATAAGCATTAGTGAACCCC | TGGCAAGTTAGGCACAGTCA | Human 1.1 kb mRNA unregulated in |
| | | | | | | | relinoic acid treated HL-60 neutro- |
| | | | | | | | philic cells |
| SGC31103 | | EST | GDB:4587265 | 0.1 | CTATGCCCAGAGATGAACAGG | TCCACTAAGGGCTATGTCGC | |
| SGC30028 | | Gene | GDB:4580505 | 0.128 | GCCAGCTTTATTGAGTAAACTTCC | CACTGGAGACTACAAGTGGTGG | Human pyruvate carbonylase precursor |
| WI-2875 | D11S4407 | STS | GDB:878546 | 0.125 | CATCCCAACCATCACTCAGT | GGGGACTAGCTTACAGATTTGA | |
| SGC36985 | | Gene | GDB:4577182 | 0.223 | AGACTACATTTTGGAACCAGTGG | TGAAAGGATATTTATAGCCTGGA | LAR-Interacting protein 1b |
| GCT16807 | D11S4270 | STS | GDB:626245 | 0.137 | GAAGGTTTTGTCCCTCGATC | TGAGGGTTGGGAAGATCATA | |
| WI-6504 | D11S3974 | EST | GDB:588142 | 0.174 | CCTTCATAGCCACACCCG | CAGCTAACTGTTGACATGCCA | |
| SGC31049 | | EST | GDB:4580093 | 0.15 | TCTTTACTGTGCTTACAACTTTCCT | CAACAGTGCAGTCGGTATCG | |
| TIGR-A002J17 | | EST | GDB:1222193 | 0.199 | AGATCAGCAAGCAGATAG | CATTCCACATGGATAGAC | NDUFV1 |
| WI-5995 | D11S2382 | EST | GDB:458683 | 0.1 | CATACCTATGAGGTGTGCTACAGG | GCATTTTCTCATCATCCTTGC | amplaxin (EMS1) |
| WI-16987 | | EST | GDB:4575848 | 0.15 | TTACAGCCACCAAGGTTTCC | AGGTGTGTGTGCCAGGTTGA | Nuclear milolic apparatus protein 1, |
| | | | | | | | NUMA |
| SGC31912 | | EST | GDB:4567868 | 0.101 | CACTGTTACTCTCATTAACTGTGAGG | TTTGATTTTGTGTCTCCAAA | |
| WI-13500 | | EST | GDB:4577893 | 0.15 | CCCCACTCCCACTTTTATTT | CCAGTCACCTTTACTAGTCCTTTG | |
| GHLC.GAAT1B01.P7933 | D11S971 | MSAT | GDB:684255 | 0.103 | AGGACACAGCCTGCATCTAG | ACCAGGCATTGCACTAAAAG | |
| SGC35519 | | Gene | GDB:4577180 | 0.134 | GATGGGTCACACTAACCTGTCA | ACATTTATATTTGGACATGCAACC | LAR-interacting protein 1a mRNA |
| WI-11974 | | EST | GDB:1222265 | 0.108 | AGCATCTTTAATGTGTCAGGCA | ATGTGCTGGGCTGGAAAG | Carniline palmiloyl transferase I |
| WI-15244 | | Gene | GDB:4574740 | 0.108 | TCACATTCAAAAATCGGCAA | CTGCCTGTGTGGTGTCGC | Beta-adrenergic receptor kinase 1, |
| | | | | | | | ADRB1 |
| WI-17496 | | EST | GDB:4583336 | 0.131 | TGTTTTATTTCTCAGTACAAAGCCA | GACCTCCTGTGACACCACG | |
| WI-9159 | D11S4381 | EST | GDB:678144 | 0.111 | CCACCAAATTATTTATAGTTCTGCG | GTAAGATTCTCCACTGTTGCACC | FGF4 |
| WI-4232 | | STS | GDB:1222250 | 0.175 | CCTATAATGGGCTGGACCAA | ACTCCTCATGTGAAGTCACCG | |
| SHGC-4167 | | EST | GDB:4566789 | 0.181 | CAGTGTGCACGTTTTCATTT | CAGCATCTTCAGCACTTACC | Human DNA helcare gen (SMBP2) |
| WI-14303 | | EST | GDB:4576938 | 0.15 | CTGCATTTATTATGAGAATCAACAG | TGCTGCTGGGAGTCAGAGTC | |
| WI-16597 | | EST | GDB:4585665 | 0.13 | CAGGGCACTGAGATACACTTACC | AAGGATCAAGCAGGCATTG | |
| RC29S1CATTFOR/RC29S1CATT | D11S970 | MSAT | GDB:191084 | 0.15 | ACACATCTCTTCTGTGCCCC | TGAACCCTGGAGGCAGAG | |
| UT979 | D11S1296 | MSAT | GDB:198525 | 0.362 | CATTCCCCAGTTTGCAGAC | GTGCTGGGATTACAGGTGT | |
| 1281/1282 | D11S1959 | EST | GDB:335216 | 0.07 | GCAGAGAAGTCCTGTTAGCC | CCATGCTAGAGAAGCACAAC | |
| D11S468 | D11S468 | STS | | 0.096 | AGTGTGGGGCAGGACCTCTG | CAGACAGATAGCCCTGGGTC | |
| D11S668 | D11S668 | STS | GDB:179349 | 0.143 | TCCCTCATCCCCTTGTCTGT | AGCCCCCCCTGGGGATAATC | |
| RH18048 | | Gene | GDB:4578253 | 0.188 | GATGCTTACCTACCACGGC | AGGATTCCTATCTGGGCTATG | Aldehyde dehydrogenase (ALDH8) |
| IGHMBP2 | | Gene | GDB:4590087 | 0.699 | TGGCAGACCATGCTCCGCCT | GAGAAGGCCGGGAGGCTCTG | Human DNA helicase gen (SMBP2) |
| NUMA | | Gene | GDB:4590244 | 0.277 | CTCCATCACAACCAGATTTGAGGCT | GGGTGTGAGCTGCTGCTGAAGG | Nuclear mitotic apparatus protein 1, |
| | | | | | | | NUMA |
| KRN1 | | Gene | GDB:4590232 | 0.228 | AGTGGGAAACCTCAGGTAGCTCCCG | CAGTTTGGCTCAGACATATGGGGGGC | High sulphur keralin, KRN |
| Cda1ff06 | D11S2302 | EST | GDB:445887 | 0.091 | CATTAAGTAGTGGGGGG | CAAAGCGACAGTGAGTTAGGG | |
| RH10753 | | Gene | GDB:4563588 | 0.194 | GGAGTAGACCATGATTACTG | CATGGTCTATTTATTCTCG | protein phosphatase 24, PP2A |
| EMS1 | | Gene | GDB:459018 | 0.64 | CGCCCTGGATCCTCACACTACA | GGGCATCAGGGGATGGGTAGA | Amplaxin |
| SHGC-11098 | DXS9736 | Gene | GDB:737674 | 0.137 | GCTCCTATCTGTGTTTTGAATGG | CCGTGGCATAAGATAAGTAAACG | Androgen Recaptor |
| INPPL1 | | Gene | GDB:4590093 | 0.382 | CTTGGAGCGCTATGAGGAGGGC | ATGGCAACTGACCTTCCGTCCTG | 51C protein inositol polyphosphate |
| | | | | | | | phosphatase-like 1 |
| RH18051 | | EST | GDB:4572859 | 0.195 | TTGGAGTCACAGGGGC | CAGCATATCCTTGGGG | NDF1 |
| Cda1cc11 | D11S2297 | EST | GDB:445869 | 0.1 | AACAAAGCTGCTTAGCACCTG | GATGAGGACCAACTGGTGAC | |
| 1249/1250 | D11S1957 | EST | GDB:335210 | 0.247 | TTTTCCAATAATGTGACTTC | CAATCCCAACCGTAACAGGC | |
| NDUFV1 | D11S2245 | EST | GDB:445695 | | CTTGATCTCGCCCAGGAAC | GCTCGCTGAAGGATGAAGAC | NDUFV1 |
| AFMb032zg5 | D11S4136 | MSAT | GDB:609546 | 0.19 | GAATCGCTTGAACCCAG | CCAGGTGGTCTTAACGG | |
| AFMa059xg9 | D11S4196 | MSAT | GDB:614025 | 0.2 | GAACGTTNTTCATGTAGGCGT | TAATGGTCGCTGTCCC | |
| Cda17c12 | D11S2288 | EST | GDB:445842 | 0.158 | AGGGAAAATGGTATGTGGGGAβ | GCAGTGTGTGAAGGCAGG | |
| SHGC-1364 | D11S951E | EST | GDB:4562765 | 0.137 | AGTGGACAAAATGAGGAAAACAGG | CCAACACAGTTTGCTCACATGCC | |
| RH17410 | | EST | GDB:4571587 | 0.126 | TGACATCTTTGCATTATGGC | AGTTATCCCACCTGATACCG | |
| RH17414 | | EST | GDB:4571595 | 0.121 | AGCTCTTGCTTCTCAGTCCA | CAAAAGTTGTTTCTGTGTTTGTTC | |
| RH17770 | | EST | GDB:4572301 | 0.267 | GCCTCTCAAAGTAGTTGGAACC | TGTGTATCCATAGTGCAAAACAG | |
| SEA | | EST | GDB:4590189 | 0.13 | CTCAAGGCCAGGCATCACT | GGACTCTTCCATGCCAGTG | S13 avlan arythrobiastosis oncogene |
| | | | | | | | homolog |
| RH10689 | | EST | GDB:4563460 | 0.107 | AATGATGATCTCAACTCTG | ACTGAAGAACTCTTGTCCT | |
| TIGR-AD06P20 | | EST | GDB:4587692 | 0.238 | GACATCTGTTAGTCTCATAATTC | GGTAACAGTGTCTTGCTT | |
| TIGR-A007D15 | | Gene | GDB:4588396 | 0.24 | CTATGTACAAAACAGGAAGAG | ATCCTAGTTTCCTCTCCTT | Merin gene (MEN1) |
| TIGR-A008B14 | | EST | GDB:4588882 | 0.141 | GTAAATGAGAAACAGACAAATGA | CTATTGGATGTGATATGTTATGG | |
| TIGR-A008K11 | | EST | GDB:4589094 | 0.203 | AAGTAGAAACAAAATGAGGGAC | CCTACCCCAAGGTAACAβ | |
| TIGR-A008P15 | | EST | GDB:4589662 | 0.182 | ACTTCCTATAAATGGAGGTGAG | GAGGAGCTTCAAGAGGAA | |
| TIGR-A008T11 | | EST | GDB:4589278 | 0.138 | CATACTCCTAGACTCAAGGAATC | GAATGATGTACATGAATTCTTTG | |
| TIGR-A008U48 | | EST | GDB:4589364 | 0.107 | GTGTTGAGGAGAAAAGCACT | CTCCCAGTAGTCACATTCC | |
| TIGR-A008X45 | | EST | GDB:4589838 | 0.242 | CAAGTTACAAATAACTTAAGCCG | CAAGACCCTATCTCTACAAAAAC | |
| SHGC-11839 | D11S4611 | Gene | GDB:740339 | 0.151 | TTTATTAGAAGTGACTCTTGGCCC | GACTACCTGCCCTCAGCTTG | Folate receptor 2 (FBP2) |
| NIB1242 | D11S4929 | EST | GDB:3888276 | 0.149 | TTCTCATGTACAAAGCGGTC | CCACTGGCTTCTCTCTTTTT | cGMP-simulated 3′, 5′-cyclic nucleotide |
| | | | | | | | phosphodiasterase PDE2A3 (PDE2A) |
| SHGC-13599 | D22S1553 | Gene | GDB:737558 | 0.147 | CACCAGAAGGTTGGGGTG | ACTATTACGACATGAACGCGG | Macrophage Migration inhibitory factor |
| SHGC-11867 | D11S4331 | Gene | GDB:674684 | 0.14 | CTCATGCTGGATGACCCC | TTGCCTTTCTTGAAACTTAATTCC | P2U Purinoceptor |
| SHGC-15349 | D12S2124 | EST | GDB:740819 | 0.141 | TCACAGCCTTCAGTCAGGG | ACATGCTGTGGCACCATG | |
| BdaB4a05 | D11S2235 | EST | GDB:445662 | 0.095 | CCTGAGCTACTGCCACAG | CCCTGACTTGGACAGTGTCC | |
| Bda99d07 | D11S2238 | EST | GDB:445674 | 0.09 | TCAGAGTCACTCCTGCCC | CAAATTCAAGCTCATCCAGACC | |
| foir1 | | Gene | GDB:197840 | 0.3 | CGGCATTTCATCCAGGAC | GGTGTAGGAGGTGCGACAAT | Folate receptor 2 (FBP2) |
| NIB1738 | D11S4284 | EST | GDB:626260 | 0.173 | TTCCATTTATTGAGCACCTG | CTTAAGCCACTGTGTTTGG | |
| WI-7351 | D11S4433 | Gene | GDB:679143 | 0.324 | CCTCCTACACCTGCAAAAGC | TGGAAGAACCCCAGAGGAC | Folate receptor 3 (FBP3) |
| WI-14325 | | EST | GDB:4578507 | 0.132 | AAAGCACAAAAGTAACAGCAACA | GTGTGTGGGCCACAATATTG | |
| WI-15192 | | EST | GDB:4575806 | 0.15 | AGAGCACCTTTCCTCAGCAC | AGAATCTCATCACAGGGGCG | |
| WI-17872 | | EST | GDB:4577492 | 0.141 | AAAAAGGACAGTGTCTAAAATTTGA | AATTGTTTTTGTTTGTTTTTTTGAGT | |
| SHGC-30732 | | EST | GDB:4567830 | 0.105 | GATTTAGGGAGTACAAGTGCGG | GGGGACAAATTATACTTTATTCAGG | |
| sISG4288 | | EST | GDB:4566057 | 0.123 | CCATCATCATATTGTGTGTGACC | TGGCTGCCCAAGAAGAAG | |
| WI-13814 | | EST | GDB:4579290 | 0.15 | TTAAGATGCCATTAAACTCATGAC | CCAAGGAGATGACCAAGTGG | DRES9 |
| WI-14122 | | Gene | GDB:4576114 | 0.126 | CCATCTCTTTTATCAGGGTTGG | CTCTGTGCAAGTAAGCATCTTACA | Human VEGF related factor isoform VRF |
| | | | | | | | 186 precursor (VRF) |
| 279/2730 | D11S4057 | EST | GDB:596509 | 0.118 | CGACTGTGTATTTTCCACAG | AGAAGCCCATATCAATGCAC | |
| SHGC-31329 | | EST | GDB:4567386 | 0.15 | AGCTTAAAGTAGGACAACCATGG | GGATGCTTCACTCCAGAAG | |
| SGC33858 | | EST | GDB:4578600 | 0.127 | TGTTGTTTATTTCCACCTGCC | AGAGTGGCTGCAGGCCAG | |
| WI-12191 | | EST | GDB:1222208 | 0.15 | TTTTTTTTTTACACGAATTTGAGG | TGAGGAAGTAAAAACAGGTCATC | |
| WI-13701 | | EST | GDB:4574892 | 0.15 | ATGAAATCTTAAGCAGAATCCCA | CACAGAGTCCCAGGGTCTGT | |
| WI-14069 | | EST | GDB:4584373 | 0.15 | AAAGGCCTTTATTTATCTCTCTCTG | GCCTCAGAGCTGGTGGGT | |
| WI-14272 | | EST | GDB:4578525 | 0.125 | GCTTCTAAGTCTTAGAGTCAGCTGG | AGCCCACAGTCAGCCTACC | |
| WI-17347 | | EST | GDB:4578523 | 0.127 | TTGGTTAAATGATGCCCAGA | TGGTCCCACTCACATCCC | |
| sISG1561 | | EST | GDB:4554415 | 0.215 | ACACAGCATGCAGGGAGAG | ATCCCTGGTGCTTAGGTGG | |
| sISG1938 | | EST | GDB:4564568 | 0.137 | GATGGAAGTAGCTCCTCTCGG | GGAAGGCCAGCAAGTACTACC | |
| sISG2759 | | EST | GDB:4565137 | 0.141 | CCGGTGCTTGGAAAGATG | GAAGTGTCTCTGTTGGGGGA | |
| RH97 | | EST | GDB:4559690 | 0.17 | TTACAGGCATGAGTCACTACGC | ACCACTCTCACAGCCCTTACA | |
| sISG4794 | | EST | GDB:4573113 | 0.141 | CCCTCCCTCCACACACAC | GCTCACTGAACTTTCAGGGC | |
| sISG4957 | | EST | GDB:4569051 | 0.171 | AGATACGGGCAAAACACTGG | GTTGAATATAGAGCAGGGCCC | |
| sISG4974 | | EST | GDB:4569063 | 0.166 | TTCTGAGGTCAGGGCTGTCT | AGCTTGGAAAATCTCGTGTCA | |
| sISG8144 | | EST | GDB:4573137 | 0.17 | ACTCAGTCCCTCCCACCC | TCCTCTCACTCCTTCCCAGA | |
| sISG9275 | | EST | GDB:4569999 | 0.19 | GTGATCACGGCTCAACCTG | TGGAGGACTGCTTGAGCC | |
| SHGC-10667 | D11S4583 | Gene | GDB:740246 | 0.277 | CTGCAGCTGCCTCAGTTTC | TCAAAAGTGCTGGTGACAGC | Human protein kinase (MLK-3) |
| SHGC-11930 | | Gene | GDB:1231223 | 0.21 | ATTTCCAGAGCCAGCTCAAA | CTTTAATGTTGTGATGACACAAAGC | FGF3 |
| SHGC-32788 | | EST | GDB:4567878 | 0.125 | GATCATGCACTGTTGACCAC | TACATTTGAAACATTTAAAACCTGA | |
| FKBP2 | | Gene | | 0.064 | AACTGAGCTGTAACCAGACTGGGA | TGGAACAGTCTGGTCCTGATGG | FK508-Binding Protein Precursor |
| | | | | | | | (FKBP-13) |
| WI-13116 | | EST | GDB:4585099 | 0.202 | TTATCCCTTTATTGTTTCTCCTTG | TGGTCACCTGTATTTATTGCTAGG | |
| MDU1 | | Gene | GDB:4590064 | 0.859 | TCTTCAAAGCCTCTGCAGTACC | CTCATCTCCAACCTGTCTAACC | 4F2 Cell-Surface Anligan Heavy Chain |
| | | | | | | | (4F2HC) |
| S453 | D11S579 | STS | GDB:196276 | 0.108 | GTGGCTGCAGCTAATGTAAGACAC | CAGCAGTAGACAATGGCGTAAGTCC | |
| STS1-cSRL-112e11 | D11S3868 | STS | GDB:547681 | 0.135 | CTGATTGAGAACCAGAACAG | TAAAGCCCTATAACCTCTCC | |
| STS1-cSRL-44a3 | D11S3830 | STS | GTC:547609 | 0.118 | TAGTAAGGGACCTTCACCAG | AGATGTTTGGTATGACTTGG | |
| STS1-cSRL-31b12 | D11S2439 | STS | GDB:459728 | 0.123 | GATGATTAAACTCTCCTGGC | GAGACAGCTAAGCACTCATG | |
| eSRL-419 | D11S1137 | STS | GDB:197824 | 0.196 | GAGGTGGTGGGCACCTGTA | AGAGGGGAGGAACACACCTT | Folate receptor 2 (FBP2) |
| SHGC-10323 | D11S4351 | Gene | GDB:676135 | 0.141 | GACCAGAGTCTGCCCAGAAG | TCCCCAGCTCTATCCCAAC | Collagen binding protein 2, colligin-2 |
| | | | | | | | gene (CBP2) |
| WI-9219 | | Gene | GDB:678179 | 0.1 | GGAGGGATGGACAAGTCTGA | GTCCAGCTCGCTGACTATCC | Retinal outer segment membrane protein |
| | | | | | | | 1, ROM1 |
| GTC ZNF | | Gene | | 0.172 | TCAAAACACAGTCATCTCCA | GCAAAGGCTTTACCATATTG | ZNF128 |
| AFMa152yn1 | D11S4087 | MSAT | GDB:603797 | 0.158 | GCTCAGCACCCCCATT | TCCCTGCTCGGGAAAC | |
| AFMb331zh5 | D11S4162 | MSAT | GDB:611241 | 0.263 | GTTCTCCAGAGAGACAGCAC | GAGAGCAACACTATTGCCC | |
| AFMb038yb9 | D11S4139 | MSAT | GDB:609621 | 0.151 | TATAGACTTCAGCCCTGCTGC | CCTCTGTAGGATGCAGTTGG | |
| AFM212xe3 | D11S1314 | MSAT | GDB:199292 | 0.209 | TTGCTACGCACTCCTCTACT | GTGAAGGCAGGAAATGTGAC | |
| WI-18813 | | EST | | 0.13 | ATCCTAGACCAGAGGAGCCC | CTCCCCCTGGTCCAGTTATT | Serial/theronine kinase |
| WI-19549 | | EST | | 0.252 | AACTTTCATTTGCCAAGGGA | AGCAGATCTGCTCTTGCGAT | |
| WI-20154 | | EST | | 0.25 | ACAGTTGTCATCGGTAGGCA | AAAAGTATGAATGGGATGGAGC | |
| WI-22393 | | EST | GDB:4583084 | 0.142 | GTGCAGGTGGCGTTTATTTT | CCCTATATCTCCGTGTGCTCC | DRES9 |
| WI-7587 | | EST | GDB:1223732 | 0.274 | GCTCTAGTGGGAAACCTCAGG | GAATTCCAGGCTCTTGCTTG | Ultra high-sulfur keralin protein (KRN1) |
| EST455579 | | EST | | 0.273 | GGTTTGGTCTCAAAGGCAAA | CCASGTACATGGTGGTCACCA | |
| WI-21134 | | EST | | 0.293 | GCTGCCTTGGAATTTCTGTT | GTGCTGTGGTGGGGAAAG | Fas-associating death domain-containing |
| | | | | | | | protein, FADD |
| WI-21698 | | EST | | 0.25 | ATTCAAGCTCATCCAGACCC | GGACTGGCCCTTTGAAACTC | |
| SHGC-7373 | D11S4567 | STS | GDB:740192 | 0.225 | ATATTGACCGTGCACAAATACG | AGACCTGGGAAAAGTGGAGAA | |
| SHGC-36533 | | STS | | 0.125 | ATTGGCAGTGGAAAATGCTT | TTAATCTTTTGTCAACTTCCTGATT | |
| ARIX | | Gene | | 0.242 | tctgtcctcctttcaccggaagc | ggataaagaaactccgctctgctggtaga | Arix homeodomain protein, neuroendoc- |
| | | | | | | | trine specific, fx factor |
| CLCI, PCR | | Gene | GDB:6262613 | | TCAGGGCCTGTGTTGCCGCACTCTG | AGCGATGTAAAGGGTACCAGTGCCG | Chloride channel current inducer, ICLN |
| | | | | | | | gene |
| B188N21-HL | STS | | | AGGCATGCAAGCTTCTTA | CCGGGAGGAGACATCTAT | | |
| B234C17-HR | | STS | | | TGGTAAGCACAGAAAATGC | AATGGATGGGGGATTATT | |
| B235G10-HR | | STS | | | CTGGACGTTATGTCTGCC | AGAGGCCCAGTCACAGAT | |
| B247F23-HR | | STS | | | ATCACTCTGAACTGCCACT | CCCTTCTGTTTTTCTGTTTT | |
| B337H24-HL | | STS | | | CAAGCTTTGAAGGAAGAG | TAGGACGTTAAGTGAGGAC | |
| B337L5-HL | | STS | | | GCTCTGCAGTGGGTAAAA | ACTCTCCAAGACTGTGCG | |
| B382N10-HR | | STS | | | CCCTTTCTGAGGCAAGAT | GACCACCTGGGAGAGAAC | |
| B1211-HR | | STS | | | CGCTATGAGTCCCATCTG | GATCAGCTGCAATGAAGG | |
| B180D17-HR | | STS | | | TTGAGTACACGGGGTGAC | CGCAGGACTGAAAGATGA | |
| B236E6-HR | | STS | | | ACCTGTCTCCTCTCCTGG | TGCTTTTCTTCTGTGGGA | |
| B27BE22-HR | | STS | | | ATGACCAGCAAGCATTGT | GTACTGGGATTACAGGCG | |
| B312F21-HR | | STS | | | GCAGAAGGTCCTTTGGAT | TTTGCAGGATTCATGCTT | |
| B337H24-HR | | STS | | | CGACATTCTTTTCTGGAGG | ACCTTTGCATGTTGGTTTT | |
| B358H9-HR | | STS | | | GCACTTTTCCTTCCTTCC | TGCTTTGCTTTCTTCTGG | |
| B148N18-HL | | STS | | | ACAGCTCCAGAGAGAAGGA | GCAGTCACTTGAAACCAGA | |
| B172N12-HL | | STS | | | AGGCATCAAGCTTTCCTT | GGTTTAGAGAACCGAGCC | |
| B172N12-HR | | STS | | | GTGGTGCTGCAAGTTACC | GGAATCCCTTTCTTTCCA | |
| B215J11-HR | | STS | | | GACCATTTGTTACGCAGC | GATGGGTGTGAATGAACAA | |
| B223E5-HR | | STS | | | CTCAAGCTTCTGTTCATGC | GCTGTGAGTGTCTTGGCT | |
| B312B3-HR | | STS | | | TACAGAAAACCGCAGCTC | GCCACCAAAGGAAGATT | |
| B328G19-HL | | STS | | | AAAAGGAGGGAATCATGG | TCACTTAGCAGGAGGCAG | |
| B328G19-HR | | STS | | | CTGAGCATCCGATGAGAC | GTGCAAAATGAGCAGCTT | |
| B32910-HL | | STS | | | TCTAACCCCTTACTGGGC | TCCTCAAACTGGGAATGA | |
| B32910-HR | | STS | | | TTTACACAGGACCAGGGA | ATCTCCCCCACTCAGAAG | |
| B368G19-HL | | STS | | | GTCCACGGGCTTTATTCT | TGAGCATAAATTTCATTAGCTG | |
| B368G19-HR | | STS | | | GGAAGAGCAAAATAAATCCA | GGTGCACAGAATTGTTCAT | |
| B36F16-HL | | STS | | | AGCACGCTTATTTCATGG | GTAACACCAGCAGGGACA | |
| B250K11-HR | | STS | | | TCCTGCTGCATTATGGAT | GGGGGTGAGAAGTAGGAA | |
| B338D17-HR | | STS | | | ATGGGGATTAAATACGGG | AGCTAGCATTGGGCTCTT | |
| B268123-HL | | STS | | | CTGAGGAGAAGAGGCTGG | CGCCTTACAAGGCAAGTA | |
| B368123-HR | | STS | | | AGGATGCTTGCTAGGGTT | CACAAGTGTCTGGAAGGC | |
| B371E15-HR | | STS | | | GGTCTCAGGAGCCCTTTA | ACATGCCACTCTTCTCACTAA | |
| B312F21-HL | | STS | | | ACTTAACCAAGGATGGGG | CAACCCAGAGCATAAGA | |
| B338D17-HL | | STS | | | TAGGCTCTGCACTCTTGG | ACCCACGGAGTCTCTCTC | |
| B369H19-HL | | STS | | | TAAAGGCGGTGAAGTGAG | CTACCGCTCTCCTAGGCT | |
| B369H19-HR | | STS | | | TGGGGCCAGATAATTCTT | CTGGTGTTTGGTGGTGTT | |
| B444M11-HR | | STS | | | AAGGAAGAGGTCACCAGG | CACAAATTCCATTTCCCA | |
| B269L23-HL | | STS | | | TCAATAGGTGATCCAACATTT | AAAGTCCCACAAAGGGTC | |
| B250K11-HL | | STS | | | GGGTAGGGGGGATCTTTTT | TGTGGAACATTCATTGGC | |
| B269L23-HR | | STS | | | GTCCTGGGAAAGATGGAA | TCAAAGCGTCTCCCATAA | |
| B364H4-HL | | STS | | | TCTTTCGCTGTACTTGGC | TGGGAGGTCAGAGTGATG | |
| B364H4-HR | | STS | | | GGACAGTGTATGTGTTGGG | AGGCAGCTGTTTTTGTGA | |
| B47303-HR | | STS | | | CTTCTTGAGTCCCGTGTG | CAACCGAGAATCCTCTAGC | |
| B180D17-HL | | STS | | | GCTGGGAGAGAATCACAA | GCTTTGCAGAAGAGACCA | |
| B200E21-HL | | STS | | | ACGCTGTCAGGTCACACT | GGAGGATGCTCAGGTGAT | |
| B200E21-HR | | STS | | | TAGGGGGATCTTTTTCCA | GAGCAATTTGAAAAGCCA | |
| B14L15-HR | | STS | | | ATGGTCCAGCTCCTCTGT | ATAGAGCACCCCATCTCC | |
| B442P6-HR | | STS | | | AACATTGCTGTTAGCCCA | GCAATCGAACAGCATTC | |
| B188N21-HR | | STS | | | ATGAGTTGGCAGCTGAAG | AATGAAGGTCTTGCCTCC | |
| GTC-ARRB1 | | Gene | | 0.067 | GAGGAGAAGATCCACAAGCG | TCTCTGGGGCATACTGAACC | Beta-arresin-1 |
| B508A5-HL | | STS | | | CTGAGCTTTTGGCACTGT | CTGTAGGTGACAGCAGG | |
| B36F16-HR | | STS | | | TGTATGAGTCTGGAGGGTGT | ACACCTGGCTGAGGAAAT | |
| B117N18-HL | | STS | | | GCAGGGGACGTGATAATA | TTTTGCTTCCTACCATGC | |
| B14L15-HL | | STS | | | AAAATTGTGAGCACCTCC | TTTATATTTAAAGTGGCTTTGTT | |
| B21K22-HL | | STS | | | GTGCAAAGCCCACAGTAT | AGGAAAATGCAAGAGCAG | |
| B21K22-HR | | STS | | | CCACTGAATTGCATACTTTG | TCTGGGTCCAGTCTGCTA | |
| B223E5-HL | | STS | | | AGATTTTGGGGAGTCAGG | GCGCTCAAGCAATTCTC | |
| B278E22-HL | | STS | | | CAAGCCCCAAAGTAGTCA | GAATCATCCAATCCACGA | |
| B4444M11-HL | | STS | | | AGCCTCCAGGTGACTACC | GAAGGACATGGTTCAGCAG | |
| B543D19-HR | | STS | | | ATGCTTTCAGCATTTTCG | TGATCCGTGGTAGGGTTA | |
| B117N18-HR | | STS | | | GTCGGATTGGTTTCACAA | TTTTATGGGAATTTCAGCC | |
| B543O19-HL | | STS | | | TTTGGAAAAGAACAGAAATGT | GGCTAGTCTTTCCTGAACC | |
| B442P6-HL | | STS | | | CCTTAATGCCCCTGATTC | GCGTTTACAAGCTGAGGA | |
| B367H4-HR | | STS | | | TCAAGCTTGCTTTCTCAA | GTAGCCCAGCAAGTGTCT | |
| B250E21-HR | | STS | | | CCTGGCTGGAGATAGGAT | CTTCCCCTCTGCCTATGT | |
| B250E21-HL | | STS | | | GGCACGTACTTCCTACCA | GGTGCTTCTTACAGGCAA | |
| B248C16-HR | | STS | | | ACCCAGGCTGGTGTGT | ACTGAGTTAATTATCACTCCCCT | |
| B248C16-HL | | STS | | | GATGCATTTTGCTTCACC | TCTGCTTTTAGAGCTGTTAGC | |
| B160D8-HR | | STS | | | TCAAGCTTCAAAGAGCAGA | GGAGTACATCCCAGGACC | |
| B539L7-HR | | STS | | | TGGTGCTTTTAAATCCAGA | CTCCCTTACTTACTTGCATTG | |
| B473O3-HL | | STS | | | TCTTCTCCCAGGGAATCT | TTTATGTCCCCTGAGCAC | |
| AFMa190xd9 | D11S4D95 | STS | GDB:606064 | 0.183 | TCCCTGGCTATCTTGAATC | CTTGACTGGGTCCACG | |
| ARRB1(2) | | STS | | | CGAGACGCCAGTAGATACCA | CATCCTCCATGCCTTTCAGT | |
| ARRB1(1) | | STS | | | AGTTCCAGAGAACGAGACGC | CTTGTCATCCTCCATGCCTT | |
| P102F3S | | STS | GDB:6054145 | | GAGCGTGAGAGGTTGAGGAG | AAACAAACTCCAGACGCACC | |
| N172A | | STS | GDB:6054146 | 0.208 | CTGAACCACTACCTGTATGACCTG | CTAACTACTTACTCCTACAGGGCCC | |
| NBDA | | STS | GDB:6054147 | 0.23 | GAAGCATTTCAATACTTTAACTG | CCACTCCAGTGCACCCAATC | |
| cCI11-44A | | STS | GDB:6054148 | 0.239 | CTTCTCCTGGCCACTCTGAC | GGTTTACCTTTGAATCCCAGC | |
| CN1677-2A | | STS | GDB:6054149 | 0.271 | TGAGGATGAATGAGCACATAGG | TTTGTGGTCCATTGAGTAGGC | |
| cCI11-5248 | | STS | GDB:6054150 | 0.221 | AGGGGAAGGAATGTGCTTGG | TTCGGCTGAGCGGGCAGTGT | |
| P117F3T | | STS | GDB:6054151 | 0.168 | ATTGAAGGTCCTCCAAAAGAATGCTG | AGAACGTCAACATATCTTTTTGGGGGACACA | |
| ARRB1(3) | | Gene | | | TTGTATTTGAGGACTTTGCTCG | CGGTACCATCCTCCTCTTCC | |
| B215J11-HL | | STS | | 0.122 | TTTTTGCCTCATCTATGCCC | GGGTGACAGAGCAAGACTCC | |
| B317G1-HR | | STS | | | TTGCTCAAGTTCTCCTGG | ACCTTGTTTTGAGGGGAG | |
| B317G1-HL | | STS | | | CTTGGCTATTTGGACAGG | GGGCATTTACTCACTTGC | |
| B292J18-HR | | STS | | | CTTGTGTCAGTTGTCAGGG | TGGAATTGTTGTGTCTTGG | |
| B292J18-HR | | STS | | | CTTGTGTCAGTTGTCAGGG | TGGAATTGTTGTGTCTTGG | |
| B10A18-HL | | STS | | | CCAGTTCCACTGGATGTT | ATGGGCTGTGTTTCTCAA | |
| B10A18-HR | | STS | | | CTGCCTATCCCTGGACTT | AGTTTGTCCCTAGTGCCC | |
| B527D12-HL | | STS | | | CAACACGTCTGACATCCAT | GGATAGTGCACACACCA | |
| B372J11-HR | | STS | | | TGGGTGGTACTATTGTTCCCAT | AGTTCCAGCCCCCTTACCAG | |
| B372J11-HL | | STS | | | GGCCACTATCATCCCTGTGT | TTTCACATGGGAGAACACG | |
| B37E17-HR(GS) | | STS | | | ACAGTGACACTAGGGACGGG | TGCCAGGATGGAGATAACAA | |
| B37E17-HL(GS) | | STS | | | CCTGTGGCACACATATCACC | ACAACCAAGAATGGAGCCAC | |
| B34F22-HR(GS) | | STS | | | TGCTGTGTAACAAGTCCCCA | TGAACGGAGGACCTACCAAG | |
| B34F22-HL(GS) | | STS | | | GCAGGGTCCGACTCACTAAG | GCTGTGAGTTCCCTTTACGC | |
| B6488P22-HR1 | | STS | | | ACAGTGGGGACAAAGACAGG | TACAGGGCACCTCCCAGTAG | |
| B82A4-HR2 | | STS | | | TCTTCTGTTAAGGTTTCCCCC | TGTCTCAAACCTCCCTCTGC | |
| B648P22-HL | | STS | | | AACATATTTCCTCCCCAGCC | CAGTCCCAGCCAATGAGAAC | |
| B82L11-HL(GS) | | STS | | | CTCCTCTGCATGGGAGAATC | AGACCTGGGACCAGTCTGTG | |
| BJ86J13-HL(GS) | | STS | | | GGGAGACGACGTCACAAGAT | TGATGTTGGGAAGATGGTGA | |
| 144A24-HL | | STS | | | CAGGCATCTTCTATGTGCCA | GGGAGGCACAAGTTCTTTCA | |
| B82L11-HR(GS) | | STS | | | ACTTCGTGGCACTGAGTGTG | CCTTTCTTACGGATGAGGCA | |
| B86J13-HR | (GS) | | STS | | | GGCTGCTGAGCTCTTCTGAT | TGGGTCTCTCTGCCTGACTT |
| B82L11-HL2(GS) | | STS | | | TCACCTACTTCCAGCTTCCG | AGACCTGGACCAGTCTGTG | |
| B82L11-HL3(GS) | | STS | | | CTCCTCTGCATGGGAGAATC | AATTCAGGAGACCTGGGACC | |

Novel STSs were developed either from publicly available genomic sequence or from sequence-derived BAC insert ends. Primers were chosen using a script which automatically performs vector and repetitive sequence masking using Cross_match (P. Green, U. of Washington) and subsequent primer picking using Primer3 (Rozen, Skaletsky (1996, 1997). Primer3 is available at www.genome.wi.mit.edu/genome_software/other/primer3.html.

Polymerase chain reaction (PCR) conditions for each primer pair were initially optimized with respect to MgCl₂ concentration. The standard buffer was 10 mM Tris-HCl (pH 8.3), 50 mM KCl, MgCl₂, 0.2 mM each dNTP, 0.2 AM each primer, 2.7 ng/μl human DNA, 0.25 units of AmpliTaq (Perkin Elmer) and MgCl₂ concentrations of 1.0 mM, 1.5 mM, 2.0 mM or 2.4 mM. Cycling conditions included an initial denaturation at 94° C. for 2 minutes followed by 40 cycles at 94° C. for 15 seconds, 55° C. for 25 seconds, and 72° C. for 25 seconds followed by a final extension at 72° C. for 3 minutes. Depending on the results from the initial round of optimization the conditions were further optimized if necessary. Variables included increasing the annealing temperature to 58° C. or 60° C., increasing the cycle number to 42 and the annealing and extension times to 30 seconds, and using AmpliTaqGold (Perkin Elmer).

BAC clones (Kim et al, Genomics, 32:213-218 (1996), Shizuya et al, Proc. Natl. Acad. Sci. USA, 89:8794-8797 (1992)) containing STS markers of interest were obtained by PCR-based screening of DNA pools from a total human BAC library purchased from Research Genetics. DNA pools derived from library plates 1-596 were used corresponding to nine genomic equivalents of human DNA. The initial screening process involved PCR reactions of individual markers against superpools, i.e., a mixture of DNA derived from all BAC clones from eight 384-well library plates. For each positive superpool, plate (8), row (16) and column (24) pools were screened to identify a unique library address. PCR products were electrophoresed in 2% agarose gels (Sigma) containing 0.5 μg/ml ethidium bromide in 1×TBE at 150 volts for 45 min. The electrophoresis units used were the Model A3-1 systems from Owl Scientific Products. Typically, gels contained 10 tiers of lanes with 50 wells/tier. Molecular weight markers (100 bp ladder, Life Technologies, Bethesda, Md.) were loaded at both ends of the gel. Images of the gels were captured with a Kodak DC40 CCD camera and processed with Kodak ID software. The gel data were exported as tab delimited text files; names of the files included information about the library screened, the gel image files and the marker screened. These data were automatically imported using a customized Perl script into Filemaker™ PRO (Claris Corp.) databases for data storage and analysis. In cases where incomplete or ambiguous clone address information was obtained, additional experiments were performed to recover a unique, complete library address.

Recovery of clonal BAC cultures from the library involved streaking out a sample from the library well onto LB agar (Maniatis et al, Molecular Cloning: A Laboratory Manual., Cold Spring Harbor Laboratory, Cold Spring Harbor, N.Y. (1982)) containing 12.5 μg/ml chloramphenicol (Sigma). Two individual colonies and a portion of the initial streak quadrant were tested with appropriate STS markers by colony PCR for verification. Positive clones were stored in LB broth containing 12.5 μg/ml chloramphenicol and 15% glycerol at −70° C.

Several different types of DNA preparation methods were used for isolation of BAC DNA. The manual alkaline lysis miniprep protocol listed below (Maniatis et al, Molecular Cloning: A Laboratory Manual, Cold Spring Harbor Laboratory, Cold Spring Harbor, N.Y. (1982)) was successfully used for most applications, i.e., restriction mapping, CHEF gel analysis, FISH mapping, but was not successfully reproducible in endsequencing. The Autogen and Qiagen protocols were used specifically for BAC DNA preparation for endsequencing purposes.

Bacteria were grown in 15 ml Terrific Broth containing 12.5 μg/ml chloramphenicol in a 50 ml conical tube at 37° C. for 20 hrs with shaking at 300 rpm. The cultures were centrifuged in a Sorvall RT 6000 D at 3000 rpm (˜1800 g) at 4° C. for 15 min. The supernatant was then aspirated as completely as possible. In some cases cell pellets were frozen at −20° C. at this step for up to 2 weeks. The pellet was then vortexed to homogenize the cells and minimize clumping. 250 μl of PI solution (50 mM glucose, 15 mM Tris-HCl, pH 8, 10 mM EDTA, and 100 Ag/ml RNase A) was added and the mixture pipetted up and down to mix. The mixture was then transferred to a 2 ml Eppendorf tube. 350 ill of P2 solution (0.2 N NaOH, 1% SDS) was then added, the mixture mixed gently and incubated for 5 min. at room temperature. 350 μl of P3 solution (3 M KOAc, pH 5.5) was added and the mixture mixed gently until a white precipitate formed. The solution was incubated on ice for 5 min. and then centrifuged at 4° C. in a microfuge for 10 min. The supernatant was transferred carefully (avoiding the white precipitate) to a fresh 2 ml Eppendorf tube, and 0.9 ml of isopropanol was added, the solution mixed and left on ice for 5 min. The samples were centrifuged for 10 min., and the supernatant removed carefully. Pellets were washed in 70% ethanol and air dried for 5 min. Pellets were resuspended in 200 μl of TE8 (10 mM Tris-HCl, pH 8.0, 1.0 mM EDTA), and RNase A (Boehringer Mannheim) added to 100 μg/ml. Samples were incubated at 37° C. for 30 min., then precipitated by addition of C₂H₃O₂Na.3H₂O to 0.5 M and 2 volumes of ethanol. Samples were centrifuged for 10 min., and the pellets washed with 70% ethanol followed by air drying and dissolving in 50 μl TE8. Typical yields for this DNA prep were 3-5 μg/15 ml bacterial culture. Ten to 15 μl were used for HindIII restriction analysis; 5 μl was used for NotI digestion and clone insert sizing by CHEF gel electrophoresis.

BACs were inoculated into 15 ml of 2×LB Broth containing 12.5 μg/ml chloramphenicol in a 50 ml conical tube. 4 tubes were inoculated for each clone. Cultures were grown overnight (˜16 hr) at 37° C. with vigorous shaking (>300 rpm). Standard conditions for BAC DNA isolation were followed as recommended by the Autogen 740 manufacturer. 3 ml samples of culture were placed into Autogen tubes for a total of 60 ml or 20 tubes per clone. Samples were dissolved finally in 100 μl TE8 with 15 seconds of shaking as part of the Autogen protocol. After the Autogen protocol was finished DNA solutions were transferred from each individual tube and pooled into a 2 ml Eppendorf tube. Tubes with large amounts of debris (carry over from the pelleting debris step) were avoided. The tubes were then rinsed with 0.5 ml of TE8 successively and this solution added to the pooled material. DNA solutions were stored at 4° C.; clumping tended to occur upon freezing at −20° C. This DNA was either used directly for restriction mapping, CHEF gel analysis or FISH mapping or was further purified as described below for use in endsequencing reactions.

The volume of DNA solutions was adjusted to 2 ml with TE8, samples were then mixed gently and heated at 650 C for 10 min. The DNA solutions were then centrifuged at 4° C. for 5 min. and the supernatants transferred to a 15 ml conical tube. The NaCl concentration was then adjusted to 0.75 M (0.3 ml of 5 M NaCl to the 2 ml sample). The total volume was then adjusted to 6 ml with Qiagen column equilibration buffer (Buffer QBT). The supernatant containing the DNA was then applied to the column and allowed to enter by gravity flow. Columns were washed twice with 10 ml of Qiagen Buffer QC. Bound DNA was then eluted with four separate 1 ml aliquots of Buffer QF kept at 65° C. DNA was precipitated with 0.7 volumes of isopropanol (2.8 ml). Each sample was then transferred to 4 individual 2.2 ml Eppendorf tubes and incubated at room temperature for 2 hr or overnight. Samples were centrifuged in a microfuige for 10 min. at 4° C. The supernatant was removed carefully and 1 ml of 70% ethanol was added. Samples were centrifuged again and because the DNA pellets were often loose at this stage, the supernatant removed carefully. Samples were centrifuged again to concentrate remaining liquid which was removed with a micropipet tip. DNA pellets were then dried in a desiccator for 10 min. 20 μl of sterile distilled and deionized H₂O was added to each tube which was then placed at 4° C. overnight. The four 20 μl samples for each clone were pooled and the tubes rinsed with another 20 μl of sterile distilled and deionized H₂O for a final volume of 100 μl. Samples were then heated at 65° C. for 5 min. and then mixed gently. Typical yields were 2-5 μg/60 ml culture as assessed by NotI digestion and comparison with uncut lambda DNA.

3 ml of LB Broth containing 12.5 μg/ml of chloramphenicol was dispensed into autoclaved Autogen tubes. A single tube was used for each clone. For inoculation, glycerol stocks were removed from −70° C. storage and placed on dry ice. A small portion of the glycerol stock was removed from the original tube with a sterile toothpick and transferred into the Autogen tube; the toothpick was left in the Autogen tube for at least two minutes before discarding. After inoculation the tubes were covered with tape making sure the seal was tight. When all samples were inoculated, the tube units were transferred into an Autogen rack holder and placed into a rotary shaker at 37° C. for 16-17 hours at 250 rpm. Following growth, standard conditions for BAC DNA preparation, as defined by the manufacturer, were used to program the Autogen. Samples were not dissolved in TE8 as part of the program and DNA pellets were left dry. When the program was complete, the tubes were removed from the output tray and 30 μl of sterile distilled and deionized H₂O was added directly to the bottom of the tube. The tubes were then gently shaken for 2-5 seconds and then covered with parafilm and incubated at room temperature for 1-3 hours. DNA samples were then transferred to an Eppendorf tube and used either directly for sequencing or stored at 4° C. for later use.

V. BAC Clone Characterization for Physical Mapping

DNA samples prepared either by manual alkaline lysis or the Autogen protocol were digested with HindIII for analysis of restriction fragment sizes. This data were used to compare the extent of overlap among clones. Typically 1-2 μg were used for each reaction. Reaction mixtures included: IX Buffer 2 (New England Biolabs), 0.1 mg/ml bovine serum albumin (New England Biolabs), 50 μg/ml RNase A (Boehringer Mannheim), and 20 units of HindIII (New England Biolabs) in a final volume of 25 μl. Digestions were incubated at 37° C. for 4-6 hours. BAC DNA was also digested with NotI for estimation of insert size by CHEF gel analysis (see below). Reaction conditions were identical to those for HindIII except that 20 units of NotI were used. Six μl of 6× Ficoll loading buffer containing bromphenol blue and xylene cyanol was added prior to electrophoresis.

HindIII digests were analyzed on 0.6% agarose (Seakem, FMC Bioproducts) in 1×TBE containing 0.5 μg/ml ethidium bromide. Gels (20 cm X 25 cm) were electrophoresed in a Model A4 electrophoresis unit (Owl Scientific) at 50 volts for 20-24 hrs. Molecular weight size markers included undigested lambda DNA, HindIII digested lambda DNA, and HaeIII digested _X174 DNA. Molecular weight markers were heated at 65° C. for 2 min. prior to loading the gel. Images were captured with a Kodak DC40 CCD camera and analyzed with Kodak ID software.

NotI digests were analyzed on a CHEF DRII (BioRad) electrophoresis unit according to the manufacturer's recommendations. Briefly, 1% agarose gels (BioRad pulsed field grade) were prepared in 0.5×TBE, equilibrated for 30 minutes in the electrophoresis unit at 14° C., and electrophoresed at 6 volts/cm for 14 hrs with circulation. Switching times were ramped from 10 sec to 20 sec. Gels were stained after electrophoresis in 0.5 μg/ml ethidium bromide. Molecular weight markers included undigested lambda DNA, HindIII digested lambda DNA, lambda ladder PFG ladder, and low range PFG marker (all from New England Biolabs).

BAC DNA prepared either by the manual alkaline lysis or Autogen protocols were labeled for FISH analysis using a Bioprime labeling kit (BioRad) according to the manufacturer's recommendation with minor modifications. Approximately 200 ng of DNA was used for each 50 μl reaction. 3 μl were analyzed on a 2% agarose gel to determine the extent of labeling. Reactions were purified using a Sephadex G50, spin column prior to in situ hybridization. Metaphase FISH was performed as described (Ma et al, Cytogenet. Cell Genet., 74:266-271 (1996)).

VI. BAC Endsequencing

The sequencing of BAC insert ends utilized DNA prepared by either of the two methods described above. The DYEnamic energy transfer primers and Dynamic Direct cycle sequencing kits from Amersham were used for sequencing reactions. Ready made sequencing mix including the M13-40 forward sequencing primer was used (Catalog # US79730) for the T7 BAC vector terminus; ready made sequencing mix (Catalog # US79530) was mixed with the M13-28 reverse sequencing primer (Catalog # US79339) for the SP6 BAC vector terminus. The sequencing reaction mixes included one of the four fluorescently labeled dye-primers, one of the four dideoxy termination mixes, dNTPs, reaction buffer, and Thermosequenase. For each BAC DNA sample, 3 μl of the BAC DNA sample was aliquoted to 4 PCR strip tubes. 2 μl of one of the four dye primer/termination mix combinations was then added to each of the four tubes. The tubes were then sealed and centrifuged briefly prior to PCR. Thermocycling conditions involved a 1 minute denaturation at 95° C., 15 second annealing at 45° C., and extension for 1 minute at 70° C. for 35 total cycles. After cycling the plates were centrifuged briefly to collect all the liquid to the bottom of the tubes. 5 μl of sterile distilled and deionized H₂O was then added into each tube, the plates sealed and centrifuged briefly again. The four samples for each BAC were then pooled together. DNA was then precipitated by adding 1.5 μl of 7.5 M NH₄OAc and 100 μl of −20° C. 100% ethanol to each tube. Samples were mixed by pipetting up and down once. The plates were then sealed and incubated on ice for 10 minutes. Plates were centrifuged in a table top Haraeus centrifuge at 4000 rpm (3,290 g) for 30 minutes at 4° C. to recover the DNA. The supernatant was removed and excess liquid blotted onto paper towels. Pellets were washed by adding 100 μl of-20° C. 70% ethanol into each tube and recentrifuging at 4000 rpm (3,290 g) for 10 minutes at 4° C. The supernatant was removed and excess liquid again removed by blotting on a paper towel. Remaining traces of liquid were removed by placing the plates upside down over a paper towel and centrifuging only until the centrifuge reached 800 rpm. Samples were then air dried at room temperature for 30 min. Tubes were capped and stored dry at −20° C. until electrophoresis. Immediately prior to electrophoresis the DNA was dissolved in 1.5 μl of Amersham loading dye. Plates were then sealed and centrifuged at 2000 rpm (825 g). The plates were then vortexed on a plate shaker for 1-2 minutes. Samples were then recentrifuged at 2000 rpm (825 g) briefly. Samples were then heated at 65° C. for 2 min. and immediately placed on ice. Standard gel electrophoresis was performed on ABI 377 fluorescent sequencers according to the manufacturer's recommendation.

VII. Sub-Cloning and Sequencing of HBM BAC DNA

The physical map of the Zmax1 gene region provides a set of BAC clones that contain within them the Zmax1 gene and the HBM gene. DNA sequencing of several of the BACs from the region has been completed. The DNA sequence data is a unique reagent that includes data that one skilled in the art can use to identify the Zmax1 gene and the HBM gene, or to prepare probes to identify the gene(s), or to identify DNA sequence polymorphisms that identify the gene(s).

BAC DNA was isolated according to one of two protocols, either a Qiagen purification of BAC DNA (Qiagen, Inc. as described in the product literature) or a manual purification which is a modification of the standard alkaline lysis/Cesium Chloride preparation of plasmid DNA (see e.g., Ausubel et al, Current Protocols in Molecular Biology, John Wiley & Sons (1997)). Briefly for the manual protocol, cells were pelleted, resuspended in GTE (50 mM glucose, 25 mM Tris-Cl (pH 8), 10 mM EDTA) and lysozyme (50 mg/ml solution), followed by NaOH/SDS (1% SDS/0.2 N NaOH) and then an ice-cold solution of 3 M KOAc (pH 4.5-4.8). RnaseA was added to the filtered supernatant, followed by Proteinase K and 20% SDS. The DNA was then precipitated with isopropanol, dried and resuspended in TE (10 mM Tris, 1 mM EDTA (pH 8.0)). The BAC DNA was further purified by Cesium Chloride density gradient centrifugation (Ausubel et al, Current Protocols in Molecular Biology, John Wiley & Sons (1997)).

Following isolation, the BAC DNA was sheared hydrodynamically using an HPLC (Hengen, Trends in Biochem. Sci., 22:273-274 (1997)) to an insert size of 2000-3000 bp. After shearing, the DNA was concentrated and separated on a standard 1% agarose gel. A single fraction, corresponding to the approximate size, was excised from the gel and purified by electroelution (Sambrook et al, Molecular Cloning: A Laboratory Manual, Cold Spring Harbor Laboratory, Cold Spring, N.Y. (1989)).

The purified DNA fragments were then blunt-ended using T4 DNA polymerase. The blunt-ended DNA was then ligated to unique BstXI-linker adapters (5′ GTCTTCACCACGGGG and 5′ GTGGTGAAGAC in 100-1000 fold molar excess). These linkers were complimentary to the BstXI-cut pMPX vectors (constructed by the inventors), while the overhang was not self-complimentary. Therefore, the linkers would not concatemerize nor would the cut-vector religate itself easily. The linker-adapted inserts were separated from the unincorporated linkers on a 1% agarose gel and purified using GeneClean (BIO 101, Inc.). The linker-adapted insert was then ligated to a modified pBlueScript vector to construct a “shotgun” subclone library. The vector contained an out-of-frame lacZ gene at the cloning site which became in-frame in the event that an adapter-dimer is cloned, allowing these to be avoided by their blue-color.

All subsequent steps were based on sequencing by ABI377 automated DNA sequencing methods. Only major modifications to the protocols are highlighted. Briefly, the library was then transformed into DH5α: competent cells (Life Technologies, Bethesda, Md., DH5 a transformation protocol). It was assessed by plating onto antibiotic plates containing ampicillin and IPTG/Xgal. The plates were incubated overnight at 37° C. Successful transformants were then used for plating of clones and picking for sequencing. The cultures were grown overnight at 37°. DNA was purified using a silica bead DNA preparation (Ng et al, Nucl. Acids Res., 24:5045-5047 (1996)) method. In this manner, 25 μg of DNA was obtained per clone.

These purified DNA samples were then sequenced using ABI dye-terminator chemistry. The ABI dye terminator sequence reads were run on ABI377 machines and the data was directly transferred to UNIX machines following lane tracking of the gels. All reads were assembled using PHRAP (P. Green, Abstracts of DOE Human Genome Program Contractor-Grantee Workshop V, January 1996, p.157) with default parameters and quality scores. The initial assembly was done at 6-fold coverage and yielded an average of 8-15 contigs. Following the initial assembly, missing mates (sequences from clones that only gave one strand reads) were identified and sequenced with ABI technology to allow the identification of additional overlapping contigs. Primers for walking were selected using a Genome Therapeutics program Pick_primer near the ends of the clones to facilitate gap closure. These walks were sequenced using the selected clones and primers. Data were reassembled with PHRAP into sequence contigs.

VIII. Gene Identification by Computational Methods

Following assembly of the BAC sequences into contigs, the contigs were subjected to computational analyses to identify coding regions and regions bearing DNA sequence similarity to known genes. This protocol included the following steps.

1. Degap the contigs: the sequence contigs often contain symbols (denoted by a period symbol) that represent locations where the individual ABI sequence reads have insertions or deletions. Prior to automated computational analysis of the contigs, the periods were removed. The original data was maintained for future reference.

2. BAC vector sequences were “masked” within the sequence by using the program cross match (Phil Green, http:chimera.biotech.washington.edu\UWGC). Since the shotgun libraries construction detailed above leaves some BAC vector in the shotgun libraries, this program was used to compare the sequence of the BAC contigs to the BAC vector and to mask any vector sequence prior to subsequent steps. Masked sequences were marked by an “X” in the sequence files, and remained inert during subsequent analyses.

3. E. coli sequences contaminating the BAC sequences were masked by comparing the BAC contigs to the entire E. coli DNA sequence.

4. Repetitive elements known to be common in the human genome were masked using cross match. In this implementation of crossmatch, the BAC sequence was compared to a database of human repetitive elements (Jerzy Jerka, Genetic Information Research Institute, Palo Alto, Calif.). The masked repeats were marked by X and remained inert during subsequent analyses.

5. The location of exons within the sequence was predicted using the MZEF computer program (Zhang, Proc. Natl. Acad. Sci., 94:565-568 (1997)).

6. The sequence was compared to the publicly available unigene database (National Center for Biotechnology Information, National Library of Medicine, 38A, 8N905, 8600 Rockville Pike, Bethesda, Md. 20894; www.ncbi.nlm.nih.gov) using the blastn2 algorithm (Altschul et al, Nucl. Acids Res., 25:3389-3402 (1997)). The parameters for this search were: E=0.05, v=50, B=50 (where E is the expected probability score cutoff, V is the number of database entries returned in the reporting of the results, and B is the number of sequence alignments returned in the reporting of the results (Altschul et al, J. Mol. Biol., 215:403-410 (1990)).

7. The sequence was translated into protein for all six reading frames, and the protein sequences were compared to a non-redundant protein database compiled from Genpept Swissprot PIR (National Center for Biotechnology Information, National Library of Medicine, 38A, 8N905, 8600 Rockville Pike, Bethesda, Md. 20894; www.ncbi.nlm.nih.gov). The parameters for this search were E=0.05, V=50, B=50, where E, V, and B are defined as above.

8. The BAC DNA sequence was compared to the database of the cDNA clones derived from direct selection experiments (described below) using blastn2 (Altschul et al, Nucl. Acids. Res., 25:3389-3402 (1997)). The parameters for this search were E=0.05, V=250, B=250, where E, V, and B are defined as above.

9. The BAC sequence was compared to the sequences of all other BACs from the HBM region on chromosome 11q12-13 using blastn2 (Altschul et al, Nucl. Acids. Res., 25:3389-3402 (1997)). The parameters for this search were E=0.05, V=50, B=50, where E, V, and B are defined as above.

10. The BAC sequence was compared to the sequences derived from the ends of BACs from the HBM region on chromosome 11q12-13 using blastn2 (Altschul et al, Nucl. Acids. Res., 25:3389-3402 (1997)). The parameters for this search were E=0.05, V=50, B=50, where E, V, and B are defined as above.

11. The BAC sequence was compared to the Genbank database (National Center for Biotechnology Information, National Library of Medicine, 38A, 8N905, 8600 Rockville Pike, Bethesda, Md. 20894; www.ncbi.nlm.nih.gov) using blastn2 (Altschul et al, Nuc. Acids. Res., 25:3389-3402 (1997)). The parameters for this search were E=0.05, V=50, B=50, where E, V, and B are defined as above.

12. The BAC sequence was compared to the STS division of Genbank database (National Center for Biotechnology Information, National Library of Medicine, 38A, 8N905, 8600 Rockville Pike, Bethesda, Md. 20894; www.ncbi.nlm.nih.gov) using blastn2 (Altschul et al, 1997). The parameters for this search were E=0.05, V=50, B=50, where E, V, and B are defined as above.

13. The BAC sequence was compared to the Expressed Sequence (EST) Tag Genbank database (National Center for Biotechnology Information, National Library of Medicine, 38A, 8N905, 8600 Rockville Pike, Bethesda, Md. 20894; www.ncbi.nlm.nih.gov) using blastn2 (Altschul et al, Nucl. Acids. Res., 25:3389-3402 (1997)). The parameters for this search were E=0.05, V=250, B=250, where E, V, and B are defined as above.

IX. Gene Identification by Direct cDNA Selection

Primary linkered cDNA pools were prepared from bone marrow, calvarial bone, femoral bone, kidney, skeletal muscle, testis and total brain. Poly (A)+ RNA was prepared from calvarial and femoral bone tissue (Chomczynski et al, Anal. Biochem., 162:156-159 (1987); D'Alessio et al, Focus, 9:1-4 (1987)) and the remainder of the mRNA was purchased from Clontech (Palo Alto, Calif.). In order to generate oligo(dT) and random primed cDNA pools from the same tissue, 2.5 μg mRNA was mixed with oligo(dT) primer in one reaction and 2.5 μg mRNA was mixed with random hexamers in another reaction, and both were converted to first and second strand cDNA according to manufacturers recommendations (Life Technologies, Bethesda, Md.). Paired phosphorylated cDNA linkers (see sequence below) were annealed together by mixing in a 1:1 ratio (10 μg each) incubated at 65° C. for five minutes and allowed to cool to room temperature.

Paired Linkers Oligo1/2

| | |
|---|---|
| OLIGO 1: | |
| 5′CTG AGC GGA ATT CGT GAG ACC3′ | (SEQ ID NO:12) |
| OLIGO 2: | |
| 5′TTG GTC TCA CGT ATT CCG CTC GA3′ | (SEQ ID NO:13) |

Paired Linkers Oligo3/4

| | |
|---|---|
| OLIGO 3: | |
| 5′CTC GAG AAT TCT GGA TCC TC3′ | (SEQ ID NO:14) |
| OLIGO 4: | |
| 5′TTG AGG ATC CAG AAT TCT CGA G3′ | (SEQ ID NO:15) |

Paired Linkers Oligo5/6

| | |
|---|---|
| OLIGO 5: | |
| 5′TGT ATG CGA ATT CGC TGC GCG3′ | (SEQ ID NO:16) |
| OLIGO 6: | |
| 5′TTC GCG CAG CGA ATT CGC ATA CA3′ | (SEQ ID NO:17) |

Paired Linkers Oligo7/8

| | |
|---|---|
| OLIGO 7: | |
| 5′GTC CAC TGA ATT CTC AGT GAG3′ | (SEQ ID NO:18) |
| OLIGO 8: | |
| 5′TTG TCA CTG AGA ATT CAG TGG AC3′ | (SEQ ID NO:19) |

Paired Linkers Oligo11/12

| | |
|---|---|
| OLIGO 11: | |
| 5′GAA TCC GAA TTC CTG GTC AGC3′ | (SEQ ID NO:20) |
| OLIGO 12: | |
| 5′TTG CTG ACC AGG AAT TCG GAT TC3′ | (SEQ ID NO:21) |

Linkers were ligated to all oligo(dT) and random primed cDNA pools (see below) according to manufacturers instructions (Life Technologies, Bethesda, Md.).

Oligo 1/2 was ligated to oligo(dT) and random primed cDNA pools prepared from bone marrow. Oligo 3/4 was ligated to oligo(dT) and random primed cDNA pools prepared from calvarial bone. Oligo 5/6 was ligated to oligo(dT) and random pruned cDNA pools prepared from brain and skeletal muscle. Oligo 7/8 was ligated to oligo(dT) and random primed cDNA pools prepared from kidney. Oligo 11/12 was ligated to oligo(dT) and random primed cDNA pools prepared from femoral bone.

The cDNA pools were evaluated for length distribution by PCR amplification using 1 μl of a 1:1, 1:10, and 1:100 dilution of the ligation reaction, respectively. PCR reactions were performed in a Perkin Elmer 9600, each 25 μl volume reaction contained 1 μl of DNA, 10 mM Tris-HCl (pH 8.3), 50 mM KCl, 1.5 mM MgCl₂, 0.01% gelatin, 200 mM each dNTPs, 10 μM primer and 1 unit Taq DNA polymerase (Perkin Elmer) and was amplified under the following conditions: 30 seconds at 94° C., 30 seconds at 60° C. and 2 minutes at 72° C. for 30 cycles. The length distribution of the amplified cDNA pools were evaluated by electrophoresis on a 1% agarose gel. The PCR reaction that gave the best representation of the random primed and oligo(dt) primed cDNA pools was scaled up so that 2-3 μg of each cDNA pool was produced. The starting cDNA for the direct selection reaction comprised of 0.5 μg of random primed cDNAs mixed with 0.5 μg of oligo(dT) primed cDNAs.

The DNA from the 54 BACs that were used in the direct cDNA selection procedure was isolated using Nucleobond AX columins as described by the manufacturer (The Nest Group, Inc.).

The BACs were pooled in equimolar amounts and 1 μg of the isolated genomic DNA was labeled with biotin 16-UTP by nick translation in accordance with the manufacturers instructions (Boehringer Mannheim). The incorporation of the biotin was monitored by methods that could be practiced by one skilled in the art (Del Mastro and Lovett, Methods in Molecular Biology, Humana Press Inc., NJ (1996)).

Direct cDNA selection was performed using methods that could be practiced by one skilled in the art (Del Mastro and Lovett, Methods in Molecular Biology, Humana Press Inc., NJ (11996)). Briefly, the cDNA pools were multiplexed in two separate reactions: In one reaction cDNA pools from bone marrow, calvarial bone, brain and testis were mixed, and in the other cDNA pools from skeletal muscle, kidney and femoral bone were mixed. Suppression of the repeats, yeast sequences and plasmid in the cDNA pools was performed to a Cot of 20. 100 ng of biotinylated BAC DNA was mixed with the suppressed cDNAs and hybridized in solution to a Cot of 200. The biotinylated DNA and the cognate cDNAs was captured on streptavidin-coated paramagnetic beads. The beads were washed and the primary selected cDNAs were eluted. These cDNAs were PCR amplified and a second round of direct selection was performed. The product of the second round of direct selection is referred to as the secondary selected material. A Galanin cDNA clone, previously shown to map to 11q12-13 (Evans, Genomics, 18:473-477 (1993)), was used to monitor enrichment during the two rounds of selection.

The secondary selected material from bone marrow, calvarial bone, femoral bone, kidney, skeletal muscle, testis and total brain was PCR amplified using modified primers of oligos 1, 3, 5, 7 and 11, shown below, and cloned into the UDG vector pAMP10 (Life Technologies, Bethesda, Md.), in accordance with the manufacturer's recommendations.

Modified Primer Sequences:

| | | |
|---|---|---|
| Oligo1-CUA: | 5′CUA CUA CUA CUA CTG AGC GGA ATT CGT GAG ACC3′ | (SEQ ID NO:22) |
| Oligo3-CUA: | 5′CUA CUA CUA CUA CTC GAG AAT TCT GGA TCC TC3′ | (SEQ ID NO:23) |
| Oligo5-CUA: | 5′CUA CUA CUA CUA TGT ATG CGA ATT CGC TGC GCG3′ | (SEQ ID NO:24) |
| Oligo7-CUA: | 5′CUA CUA CUA CUA GTC CAC TGA ATT CTC AGT GAG3′ | (SEQ ID NO:25) |
| Oligo11-CUA: | 5′CUA CUA CUA CUA GAA TCC GAA TTC CTG GTC AGC3′ | (SEQ ID NO:26) |

The cloned secondary selected material, from each tissue source, was transformed into MAX Efficiency DH5 a Competent Cells (Life Technologies, Bethesda, Md.) as recommended by the manufacturer. 384 colonies were picked from each transformed source and arrayed into four 96 well microtiter plates. All secondarily selected cDNA clones were sequenced using M13 dye primer terminator cycle sequencing kit (Applied Biosystems), and the data collected by the ABI 377 automated fluorescence sequencer (Applied Biosystems). All sequences were analyzed using the BLASTN, BLASTX and FASTA programs (Altschul et al, J. Mol. Biol., 215:403-410 (1990), Altschul et al, Nucl. Acids. Res., 25:3389-3402 (1997)). The cDNA sequences were compared to a database containing sequences derived from human repeats, mitochondrial DNA, ribosomal RNA, E. coli DNA to remove background clones from the dataset using the program cross_match. A further round of comparison was also performed using the program BLASTN2 against known genes (Genbank) and the BAC sequences from the HBM region. Those cDNAs that were >90% homologous to these sequences were filed according to the result and the data stored in a database for further analysis. cDNA sequences that were identified but did not have significant similarity to the BAC sequences from the HBM region or were eliminated by crossmatch were hybridized to nylon membranes which contained the BACs from the HBM region, to ascertain whether they hybridized to the target.

Hybridization analysis was used to map the cDNA clones to the BAC target that selected them. The BACs that were identified from the HBM region were arrayed and grown into a 96 well microtiter plate. LB agar containing 25 μg/ml kanamycin was poured into 96 well microtiter plate lids. Once the agar had solidified, pre-cut Hybond N+ nylon membranes (Amersham) were laid on top of the agar and the BACs were stamped onto the membranes in duplicate using a hand held 96 well replica plater (V&P Scientific, Inc.). The plates were incubated overnight at 37° C. The membranes were processed according to the manufacturers recommendations.

The cDNAs that needed to be mapped by hybridization were PCR amplified using the relevant primer (oligos 1, 3, 5, 7 and 11) that would amplify that clone. For this PCR amplification, the primers were modified to contain a Tinkered digoxigenin molecule at the 5′ of the oligonucleotide. The PCR amplification was performed under the same conditions as described in Preparation of cDNA Pools (above). The PCR products were evaluated for quality and quantity by electrophoresis on a 1% agarose gel by loading 5 μl of the PCR reaction. The nylon membranes containing the stamped BACs were individually pre-hybridized in 50 ml conical tubes containing 10 ml of hybridization solution (5×SSPE, 0.5× Blotto, 2.5% SDS and 1 mM EDTA (pH 8.0)). The 50 ml conical tubes were placed in a rotisserie oven (Robbins Scientific) for 2 hours at 65° C. Twenty-five ng of each cDNA probe was denatured and added into individual 50 ml conical tubes containing the nylon membrane and hybridization solution. The hybridization was performed overnight at 65° C. The filters were washed for 20 minutes at 65° C. in each of the following solutions: 3×SSPE, 0.1% SDS; 1×SSPE, 0.1% SDS and 0.1×SSPE, 0.1% SDS.

The membranes were removed from the 50 ml conical tubes and placed in a dish. Acetate sheets were placed between each membrane to prevent them from sticking to each other. The incubation of the membranes with the Anti-DIG-AP and CDP-Star was performed according to manufacturers recommendations (Boehringer Mannheim). The membranes were wrapped in Saran wrap and exposed to Kodak Bio-Max X-ray film for 1 hour.

X. cDNA Cloning and Expression Analysis

To characterize the expression of the genes identified by direct cDNA selection and genomic DNA sequencing in comparison to the publicly available databases, a series of experiments were performed to further characterize the genes in the HBM region. First, oligonucleotide primers were designed for use in the polymerase chain reaction (PCR) so that portions of a cDNA, EST, or genomic DNA could be amplified from a pool of DNA molecules (a cDNA library) or RNA population (RT-PCR and RACE). The PCR primers were used in a reaction containing genomic DNA to verify that they generated a product of the size predicted based on the genomic (BAC) sequence. A number of cDNA libraries were then examined for the presence of the specific cDNA or EST. The presence of a fragment of a transcnrption unit in a particular cDNA library indicates a high probability that additional portions of the same transcription unit will be present as well.

A critical piece of data that is required when characterizing novel genes is the length, in nucleotides, of the processed transcript or messenger RNA (mRNA). One skilled in the art primarily determines the length of an mRNA by Northern blot hybridization (Sambrook et al, Molecular Cloning: A Laboratory Manual, Cold Spring Harbor Laboratory, Cold Spring Harbor N.Y. (1989)). Groups of ESTs and direct-selected cDNA clones that displayed significant sequence similarity to sequenced BACs in the critical region were grouped for convenience into approximately 30 kilobase units. Within each 30 kilobase unit there were from one up to fifty ESTs and direct-selected cDNA clones which comprised one or more independent transcription units. One or more ESTs or direct-selected cDNAs were used as hybridization probes to determine the length of the mRNA in a variety of tissues, using commercially available reagents (Multiple Tissue Northern blot; Clontech, Palo Alto, Calif.) under conditions recommended by the manufacturer.

Directionally cloned cDNA libraries from femoral bone, and calvarial bone tissue were constructed by methods familiar to one skilled in the art (for example, Soares in Automated DNA Sequencing and Analysis, Adams, Fields and Venter, Eds., Academic Press, NY, pages 110-114 (1994)). Bones were initially broken into fragments with a hammer, and the small pieces were frozen in liquid nitrogen and reduced to a powder in a tissue pulverizer (Spectrum Laboratory Products). RNA was extracted from the powdered bone by homogenizing the powdered bone with a standard Acid Guanidinium Thiocyanate-Phenol-Chloroform extraction buffer (e.g. Chomczynski and Sacchi, Anal. Biochem., 162:156-159 (1987)) using a polytron homogenizer (Brinkman Instruments). Additionally, human brain and lung total RNA was purchased from Clontech. PolyA RNA was isolated from total RNA using dynabeads-dT according to the manufacturer's recommendations (Dynal, Inc.).

First strand cDNA synthesis was initiated using an oligonucleotide primer with the sequence: 5′-AACTGGAAGAATTCGCGGCCGCAGGAATTTTTTTTT TTTTTTTTT-3′ (SEQ ID NO:27). This primer introduces a NotI restriction site (underlined) at the 3′ end of the cDNA. First and second strand synthesis were performed using the “one-tube” cDNA synthesis kit as described by the anufacturer (Life Technologies, Bethesda, Md.). Double stranded cDNAs were treated with T4 polynucleotide kinase to ensure that the ends of the molecules were blunt (Soares in Automated DNA Sequencing and Analysis, Adams, Fields and Venter, Eds., Academic Press, NY, pages 110-114 (1994)), and the blunt ended cDNAs were then size selected by a Biogel column (Huynh et al in DNA Cloning, Vol. 1, Glover, Ed., IRL Press, Oxford, pages 49-78 (1985)) or with a size-sep 400 sepharose column (Pharmacia, catalog # 27-5105-01). Only cDNAs of 400 base pairs or longer were used in subsequent steps. EcoRI adapters (sequence: 5′ OH-AATTCGGCACGAG-OH 3′ (SEQ ID NO:28), and 5′ p-CTCGTGCCG-OH 3′ (SEQ ID NO:29)) were then ligated to the double stranded cDNAs by methods familiar to one skilled in the art (Soares, 1994). The EcoRI adapters were then removed from the 3′ end of the cDNA by digestion with NotI (Soares, 1994). The cDNA was then ligated into the plasmid vector pBluescript II KS+ (Stratagene, La Jolla, Calif.), and the ligated material was transformed into E. coli host DH10B or DH12S by electroporation methods familiar to one skilled in the art (Soares, 1994). After growth overnight at 37° C., DNA was recovered from the E. coli colonies after scraping the plates by processing as directed for the Mega-prep kit (Qiagen, Chatsworth, Calif.). The quality of the cDNA libraries was estimated by counting a portion of the total numbers of primary transformants and determining the average insert size and the percentage of plasmids with no cDNA insert. Additional cDNA libraries (human total brain, heart, kidney, leukocyte, and fetal brain) were purchased from Life Technologies, Bethesda, Md.

cDNA libraries, both oligo (dT) and random hexamer (N₆) primed, were used for isolating cDNA clones transcribed within the HBM region: human bone, human brain, human kidney and human skeletal muscle (all cDNA libraries were made by the inventors, except for skeletal muscle (dT) and kidney (dT) cDNA libraries). Four 10×10 arrays of each of the cDNA libraries were prepared as follows: the cDNA libraries were titered to 2.5×10⁶ using primary transformants. The appropriate volume of frozen stock was used to inoculate 2 L of LB/ampicillin (100 mg/ml). This inoculated liquid culture was aliquotted into 400 tubes of 4 ml each. Each tube contained approximately 5000 cfu. The tubes were incubated at 30° C. overnight with gentle agitation. The cultures were grown to an OD of 0.7-0.9. Frozen stocks were prepared for each of the cultures by aliquotting 100 μl of culture and 300 μl of 80% glycerol. Stocks were frozen in a dry ice/ethanol bath and stored at −70° C. The remaining culture was DNA prepared using the Qiagen (Chatsworth, Calif.) spin miniprep kit according to the manufacturer's instructions. The DNAs from the 400 cultures were pooled to make 80 column and row pools. The cDNA libraries were determined to contain HBM cDNA clones of interest by PCR. Markers were designed to amplify putative exons. Once a standard PCR optimization was performed and specific cDNA libraries were determined to contain cDNA clones of interest, the markers were used to screen the arrayed library. Positive addresses indicating the presence of cDNA clones were confirmed by a second PCR using the same markers.

Once a cDNA library was identified as likely to contain cDNA clones corresponding to a specific transcript of interest from the HBM region, it was manipulated to isolate the clone or clones containing cDNA inserts identical to the EST or direct-selected cDNA of interest. This was accomplished by a modification of the standard “colony screening” method (Sambrook et al, Molecular Cloning: A Laboratory Manual, Cold Spring Harbor Laboratory, Cold Spring Harbor N.Y. (1989)). Specifically, twenty 150 mm LB+ampicillin agar plates were spread with 20,000 colony forming units (cfu) of cDNA library and the colonies allowed to grow overnight at 37° C. Colonies were transferred to nylon filters (Hybond from Amersham, or equivalent) and duplicates prepared by pressing two filters together essentially as described (Sambrook et al, Molecular Cloning: A Laboratory Manual, Cold Spring Harbor Laboratory, Cold Spring Harbor N.Y. (1989)). The “master” plate was then incubated an additional 6-8 hours to allow the colonies to grow back. The DNA from the bacterial colonies was then affixed to the nylon filters by treating the filters sequentially with denaturing solution (0.5 N NaOH, 1.5 M NaCl) for two minutes, neutralization solution (0.5 M Tris-Cl pH 8.0, 1.5 M NaCl) for two minutes (twice). The bacterial colonies were removed from the filters by washing in a solution of 2×SSC/0.1% SDS for one minute while rubbing with tissue paper. The filters were air dried and baked under vacuum at 80° C. for 1-2 hours.

A cDNA hybridization probe was prepared by random hexamer labeling (Fineberg and Vogelstein, Anal. Biochem., 132:6-13 (1983)) or by including gene-specific primers and no random hexamers in the reaction (for small fragments). Specific activity was calculated and was >5×10⁸ cpm/10⁸ μg of cDNA. The colony membranes were then prewashed in 10 mM Tris-Cl pH 8.0, 1 M NaCl, 1 mM EDTA, 0.1% SDS for 30 minutes at 55° C. Following the prewash, the filters were prehybridized in >2 ml/filter of 6×SSC, 50% deionized formamide, 2% SDS, 5× Denhardt's solution, and 100 mg/ml denatured salmon sperm DNA, at 42° C. for 30 minutes. The filters were then transferred to hybridization solution (6×SSC, 2% SDS, 5× Denhardt's, 100 mg/ml denatured salmon sperm DNA) containing denatured α³²P-dCTP-labeled cDNA probe and incubated at 42° C. for 16-18 hours.

After the 16-18 hour incubation, the filters were washed under constant agitation in 2×SSC, 2% SDS at room temperature for 20 minutes, followed by two washes at 65° C. for 15 minutes each. A second wash was performed in 0.5×SSC, 0.5% SDS for 15 minutes at 65° C. Filters were then wrapped in plastic wrap and exposed to radiographic film for several hours to overnight. After film development, individual colonies on plates were aligned with the autoradiograph so that they could be picked into a 1 ml solution of LB Broth containing ampicillin. After shaking at 37° C. for 1-2 hours, aliquots of the solution were plated on 150 mm plates for secondary screening. Secondary screening was identical to primary screening (above) except that it was performed on plates containing 250 colonies so that individual colonies could be clearly identified for picking.

After colony screening with radiolabeled probes yielded cDNA clones, the clones were characterized by restriction endonuclease cleavage, PCR, and direct sequencing to confirm the sequence identity between the original probe and the isolated clone. To obtain the full-length cDNA, the novel sequence from the end of the clone identified was used to probe the library again. This process was repeated until the length of the cDNA cloned matches that estimated to be full-length by the northern blot analysis.

RT-PCR was used as another method to isolate full length clones. The cDNA was synthesized and amplified using a “Superscript One Step RT-PCR” kit (Life Technologies, Gaithersburg, Md.). The procedure involved adding 1.5 μg of RNA to the following: 25 μl of reaction mix provided which is a proprietary buffer mix with MgSO₄ and dNTP's, 1 μl sense primer (10 μM) and 1 S11 anti-sense primer (10 μM), 1 III reverse transcriptase and Taq DNA polymerase mix provided and autoclaved water to a total reaction mix of 50 pl. The reaction was then placed in a thermocycler for 1 cycle at 50° C. for 15 to 30 minutes, then 94° C. for 15 seconds, 55-60° C. for 30 seconds and 68-72° C. for 1 minute per kilobase of anticipated product and finally 1 cycle of 72° C. for 5-10 minutes. The sample was analyzed on an agarose gel. The product was excised from the gel and purified from the gel (GeneClean, Bio 101). The purified product was cloned in pCTNR (General Contractor DNA Cloning System, 5 Prime-3 Prime, Inc.) and sequenced to verify that the clone was specific to the gene of interest.

Rapid Amplification of cDNA ends (RACE) was performed following the manufacturer's instructions using a Marathon cDNA Amplification Kit (Clontech, Palo Alto, Calif.) as a method for cloning the 5′ and 3′ ends of candidate genes. cDNA pools were prepared from total RNA by performing first strand synthesis, where a sample of total RNA sample was mixed with a modified oligo (dT) primer, heated to 70° C., cooled on ice and followed by the addition of: 5×first strand buffer, 10 mM dNTP mix, and AMV Reverse Transcriptase (20 U/μl). The tube was incubated at 42° C. for one hour and then the reaction tube was placed on ice. For second strand synthesis, the following components were added directly to the reaction tube: 5× second strand buffer, 10 mM dNTP mix, sterile water, 20× second strand enzyme cocktail and the reaction tube was incubated at 16° C. for 1.5 hours. T4 DNA Polymerase was added to the reaction tube and incubated at 16° C. for 45 minutes. The second-strand synthesis was terminated with the addition of an EDTA/Glycogen mix. The sample was subjected to a phenol/chloroform extraction and an ammonium acetate precipitation. The cDNA pools were checked for quality by analyzing on an agarose gel for size distribution. Marathon cDNA adapters (Clontech) were then ligated onto the cDNA ends. The specific adapters contained priming sites that allowed for amplification of either 5′ or 3′ ends, depending on the orientation of the gene specific primer (GSP) that was chosen. An aliquot of the double stranded cDNA was added to the following reagents: 10 μM Marathon cDNA adapter, 5×DNA ligation buffer, T4 DNA ligase. The reaction was incubated at 16° C. overnight. The reaction was heat inactivated to terminate the reaction. PCR was performed by the addition of the following to the diluted double stranded cDNA pool: 10× cDNA PCR reaction buffer, 10 μM dNTP mix, 10,LM GSP, 10 μM API primer (kit), 50×Advantage cDNA Polymerase Mix. Thermal Cycling conditions were 94° C. for 30 seconds, 5 cycles of 94° C. for 5 seconds, 72° C. for 4 minutes, 5 cycles of 94° C. for 5 seconds, 70° C. for 4 minutes, 23 cycles of 94° C. for 5 seconds, 68° C. for 4 minutes. After the first round of PCR was performed using the GSP to extend to the end of the adapter to create the adapter primer binding site, exponential amplification of the specific cDNA of interest was observed. Usually a second nested PCR is performed to confirm the specific cDNA. The RACE product was analyzed on an agarose gel and then excised and purified from the gel (GeneClean, BIO 101). The RACE product was then cloned into pCTNR (General Contractor DNA Cloning System, 5′-3′, Inc.) and the DNA sequence determined to verify that the clone is specific to the gene of interest.

XI. Mutation Analysis

Comparative genes were identified using the above procedures and the exons from each gene were subjected to mutation detection analysis. Comparative DNA sequencing was used to identify polymorphisms in HBM candidate genes from chromosome 11q12-13. DNA sequences for candidate genes were amplified from patient lymphoblastoid cell lines.

The inventors developed a method based on analysis of direct DNA sequencing of PCR products amplified from candidate regions to search for the causative polymorphism. The procedure consisted of three stages that used different subsets of HBM family to find segregating polymorphisms and a population panel to assess the frequency of the polymorphisms. The family resources result from a single founder leading to the assumption that all affected individuals will share the same causative polymorphism.

Candidate regions were first screened in a subset of the HBM family consisting of the proband, daughter, and her mother, father and brother. Monochromosomal reference sequences were produced concurrently and used for comparison. The mother and daughter carried the HBM polymorphism in this nuclear family, providing the ability to monitor polymorphism transmission. The net result is that two HBM chromosomes and six non-HBM chromosomes were screened. This allowed exclusion of numerous frequent alleles. Only alleles exclusively present in the affected individuals passed to the next level of analysis.

Polymorphisms that segregated exclusively with the HBM phenotype in this original family were then re-examined in an extended portion of the HBM pedigree consisting of two additional nuclear families. These families consisted of five HBM and three unaffected individuals. The HBM individuals in this group included the two critical crossover individuals, providing the centromeric and telomeric boundaries of the critical region. Tracking the heredity of polymorphisms between these individuals and their affected parents allowed for further refining of the critical region. This group brought the total of HBM chromosomes screened to seven and the total of non-HBM chromosomes to seventeen.

When a given polymorphism continued to segregate exclusively with the HBM phenotype in the extended group, a population panel was then examined. This panel of 84 persons consisted of 42 individuals known to have normal bone mineral density and 42 individuals known to be unrelated but with untyped bone mineral density. Normal bone mineral density is within two standard deviations of BMD Z score 0. The second group was from the widely used CEPH panel of individuals. Any segregating polymorphisms found to be rare in this population were subsequently examined on the entire HBM pedigree and a larger population.

Polymerase chain reaction (PCR) was used to generate sequencing templates from the HBM family's DNA and monochromosomal controls. Enzymatic amplification of genes within the HBM region on 11q12-13 was accomplished using the PCR with oligonucleotides flanking each exon as well as the putative 5′ regulatory elements of each gene. The primers were chosen to amplify each exon as well as 15 or more base pairs within each intron on either side of the splice. All PCR primers were made as chimeras to facilitate dye primer sequencing. The M13-21F (5′-GTA A CGA CGG CCA GT-3′) (SEQ ID NO:30) and −28REV (5′-AAC AGC TAT GAC CAT G-3′) (SEQ ID NO:31) primer binding sites were built on to the 5′ end of each forward and reverse PCR primer, respectively, during synthesis. 150 ng of genomic DNA was used in a 50 μl PCR with 2 U AmpliTaq, 500 nM primer and 125 nM dNTP; Buffer and cycling conditions-were specific to each primer set. TaqStart antibody (Clontech) was used for hot start PCR to minimize primer dimer formation. 10% of the product was examined on an agarose gel. The appropriate samples were diluted 1:25 with deionized water before sequencing.

Each PCR product was sequenced according to the standard Energy Transfer primer (Amersham) protocol. All reactions took place in 96 well trays. 4 separate reactions, one each for A, C, G and T were performed for each template. Each reaction included 2 μl of the sequencing reaction mix and 3 μl of diluted template. The plates were then heat sealed with foil tape and placed in a thermal cycler and cycled according to the manufacturer's recommendation. After cycling, the 4 reactions were pooled. 3 μl of the pooled product was transferred to a new 96 well plate and 1 μl of the manufacturer's loading dye was added to each well. All 96 well pipetting procedures occurred on a Hydra 96 pipetting station (Robbins Scientific, USA). 1 μl of pooled material was directly loaded onto a 48 lane gel running on an ABI 377 DNA sequencer for a 10 hour, 2.4 kV run.

Polyphred (University of Washington) was used to assemble sequence sets for viewing with Consed (University of Washington). Sequences were assembled in groups representing all relevant family members and controls for a specified target region. This was done separately for each of the three stages. Forward and reverse reads were included for each individual along with reads from the monochromosomal templates and a color annotated reference sequence. Polyphred indicated potential polymorphic sites with a purple flag. Two readers independently viewed each assembly and assessed the validity of the purple-flagged sites.

A total of 23 exons present in the mature mRNA and several other portions of the primary transcript were evaluated for heterozygosity in the nuclear family of two HBM-affected and two unaffected individuals. 25 SNPs were identified, as shown in the table below.

TABLE 4
Single Nucleotide Polymorphisms in the Zmax1 Gene and Environs

| Exon Name | Location | Base Change |
|---|---|---|
| b200e21-h_Contig1_1.nt | 69169 (309G) | C/A |
| b200e21-h_Contig4_12.nt | 27402 (309G) | A/G |
| b200e21-h_Contig4_13.nt | 27841 (309G) | T/C |
| b200e21-h_Contig4_16.nt | 35600 (309G) | A/G |
| b200e21-h_Contig4_21.nt | 45619 (309G) | G/A |
| b200e21-h_Contig4_22.nt-a | 46018 (309G) | T/G |
| b200e21-h_Contig4_22.nt-b | 46093 (309G) | T/G |
| b200e21-h_Contig4_22.nt-c | 46190 (309G) | A/G |
| b200e21-h_Contig4_24.nt-a | 50993 (309G) | T/C |
| b200e21-h_Contig4_24.nt-b | 51124 (309G) | C/T |
| b200e21-h_Contig4_25.nt | 55461 (309G) | C/T |
| b200e21-h_Contig4_33.nt-a | 63645 (309G) | C/A |
| b200e21-h_Contig4_33.nt-b | 63646 (309G) | A/C |
| b200e21-h_Contig4_61.nt | 24809 (309G) | T/G |
| b200e21-h_Contig4_62.nt | 27837 (309G) | T/C |
| b200e21-h_Contig4_63.nt-a | 31485 (309G) | C/T |
| b200e21-h_Contig4_63.nt-b | 31683 (309G) | A/G |
| b200e21-h_Contig4_9.nt | 24808 (309G) | T/G |
| b527d12-h_Contig030g_1.nt-a | 31340 (308G) | T/C |
| b527d12-h_Contig030g_1.nt-b | 32538 (308G) | A/G |
| b527d12-h_Contig080C_2.nt | 13224 (308G) | A/G |
| b527d12-h_Contig087C_1.nt | 21119 (308G) | C/A |
| b527d12-h_Contig087C_4.nt | 30497 (308G) | G/A |
| b527d12-h_Contig088C_4.nt | 24811 (309G) | A/C |
| b527d12-h_Contig089_1HP.nt | 68280 (309G) | G/A |

In addition to the polymorphisms presented in Table 4, two additional polymorphisms can also be present in SEQ ID NO:2. These is a change at position 2002 of SEQ ID NO:2. Either a guanine or an adenine can appear at this position. This polymorphism is silent and is not associated with any change in the amino acid sequence. The second change is at position 4059 of SEQ ID NO:2 corresponding in a cytosine (C) to thymine (T) change. This polymorphism results in a corresponding amino acid change from a valine (to an alanine (A). Other polymorphisms were found in the candidate gene exons and adjacent intron sequences. Any one or combination of the polymorphisms listed in Table 4 or the two discussed above could also have a minor effect on bone mass when present in SEQ ID NO:2.

The present invention encompasses the nucleic acid sequences having the nucleic acid sequence of SEQ ID NO: 1 with the above-identified point mutations.

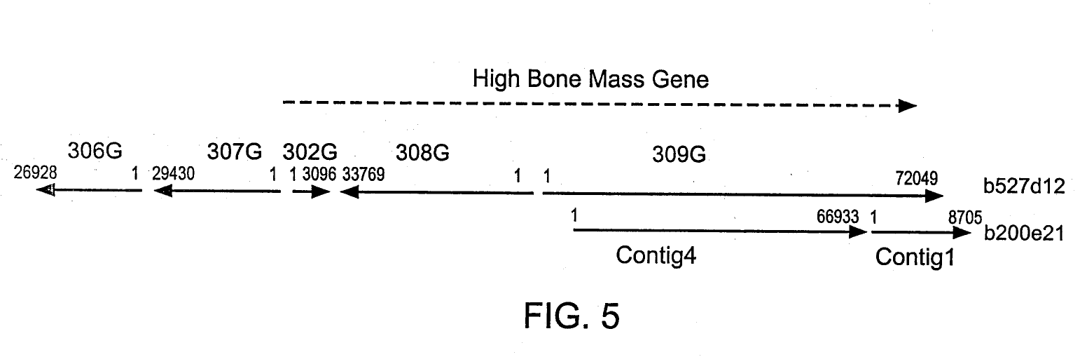

Preferably, the present invention encompasses the nucleic acid of SEQ ID NO: 2. Specifically, a base-pair substitution changing G to T at position 582 in the coding sequence of Zmax1 (the HBM gene) was identified as heterozygous in all HBM individuals, and not found in the unaffected individuals (i.e., b527d12-h_Contig087C_{—}1.nt). FIG. 5 shows the order of the contigs in B527D12. The direction of transcription for the HBM gene is from left to right. The sequence of contig308G of B527D12 is the reverse complement of the coding region to the HBM gene. Therefore, the relative polymorphism in contig 308G shown in Table 4 as a base change substitution of C to A is the complement to the G to T substitution in the HBM gene. This mutation causes a substitution of glycine 171 with valine (G171V).

The HBM polymorphism was confirmed by examining the DNA sequence of different groups of individuals. In all members of the HBM pedigree (38 individuals), the HBM polymorphism was observed in the heterozygous form in affected (i.e., elevated bone mass) individuals only (N=18). In unaffected relatives (N=20) (BMDZ<2.0) the HBM polymorphism was never observed. To determine whether this polymorphism was ever observed in individuals outside of the HBM pedigree, 297 phenotyped individuals were characterized at the site of the HBM gene. None were heterozygous at the site of the HBM polymorphism. In an unphenotyped control group, none of 64 individuals were observed to be heterozygous at position 582. Taken together, these data prove that the polymorphism observed in the kindred displaying the high bone mass phenotype is strongly correlated with the G→T polymorphism at position 582 of Zmax1. Furthermore, these results coupled with the ASO results described below, establish that the HBM polymorphism genetically segregates with the HBM phenotype, and that both the HBM polymorphism and phenotype are rare in the general population.

XII. Allele Specific Oligonucleotide (ASO) Analysis

The amplicon containing the HBM1 polymorphism was PCR amplified using primers specific for the exon of interest. The appropriate population of individuals was PCR amplified in 96 well microtiter plates as follows. PCR reactions (20 μl) containing 1× Promega PCR buffer (Cat. # M1883 containing 1.5 MM MgCl₂), 100 mM dNTP, 200 nM PCR primers (1863F: CCAAGTTCTGAGAAGTCC and 1864R: AATACCTGAAACCATACCTG), 1 U Amplitaq, and 20 ng of genomic DNA were prepared and amplified under the following PCR conditions: 94° C., 1 minute, (94° C., 30 sec.; 58° C., 30 sec.; 72° C., 1 min.) X35 cycles), 72° C., 5′, 4° C., hold. Loading dye was then added and 10 μl of the products was electrophoresed on 1.5% agarose gels containing 1 μg/ml ethidium bromide at 100-150 V for 5-10 minutes. Gels were treated 20 minutes in denaturing solution (1.5 M NaCl, 0.5 N NaOH), and rinsed briefly with water. Gels were then neutralized in 1 M Tris-HCl, pH 7.5, 1.5 M NaCl, for 20 minutes and rinsed with water. Gels were soaked in 10×SSC for 20 minutes and blotted onto nylon transfer membrane (Hybond N+-Amersham) in 10×SSC overnight. Filters were the rinsed in 6×SSC for 10 minutes and UV crosslinked.

The allele specific oligonucleotides (ASO) were designed with the polymorphism approximately in the middle. Oligonucleotides were phosphate free at the 5′end and were purchased from Gibco BRL. Sequences of the oligonucleotides are:

| | |
|---|---|
| 2326 Zmax1.ASO.g: | AGACTGGGGTGAGACGC |
| 2327 Zmax1.ASO.t: | CAGACTGGGTTGAGACGCC |

The polymorphic nucleotides are underlined. To label the oligos, 1.5 ill of 1 μg/μl ASO oligo (2326.Zmax1.ASO.g or 2327.Zmax1.ASO.t), 11 μL ddH₂O, 2 μl 10× kinase forward buffer, 5 μl γ³²p-ATp (6000 Ci/mMole), and 1 pi T4 polynucleotide kinase (10 U/μl) were mixed, and the reaction incubated at 37° C. for 30-60 minutes. Reactions were then placed at 95° C. for 2 minutes and 30 ml H₂O was added. The probes were purified using a G25 microspin column (Pharmacia).

Blots were prehybridized in 10 ml 5×SSPE, 5× Denhardt's, 2% SDS, and 100 pug/ml, denatured, sonicated salmon sperm DNA at 40° C. for 2 hr. The entire reaction mix of kinased oligo was then added to 10 ml fresh hybridization buffer (5×SSPE, 5× Denhardt's, 2% SDS) and hybridized at 40° C. for at least 4 hours to overnight.

All washes done in 5×SSPE, 0.1% SDS. The first wash was at 45° C. for 15 minutes; the solution was then changed and the filters washed 50° C. for 15 minutes. Filters were then exposed to Kodak biomax film with 2 intensifying screens at −70° C. for 15 minutes to 1 hr. If necessary the filters were washed at 55° C. for 15 minutes and exposed to film again. Filters were stripped by washing in boiling 0.1×SSC, 0.10% SDS for 10 minutes at least 3 times.

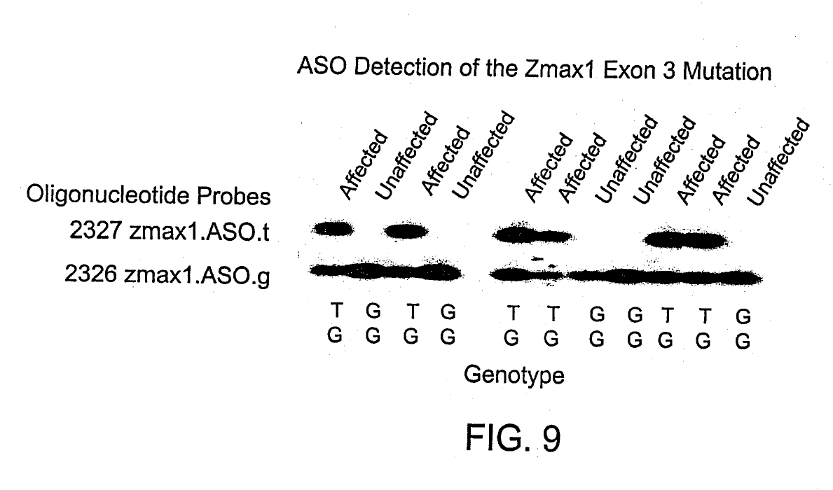

The two films that best captured the allele specific assay with the 2 ASOs were converted into digital images by scanning them into Adobe PhotoShop. These images were overlaid against each other in Graphic Converter and then scored and stored in FileMaker Pro 4.0 (see FIG. 9).

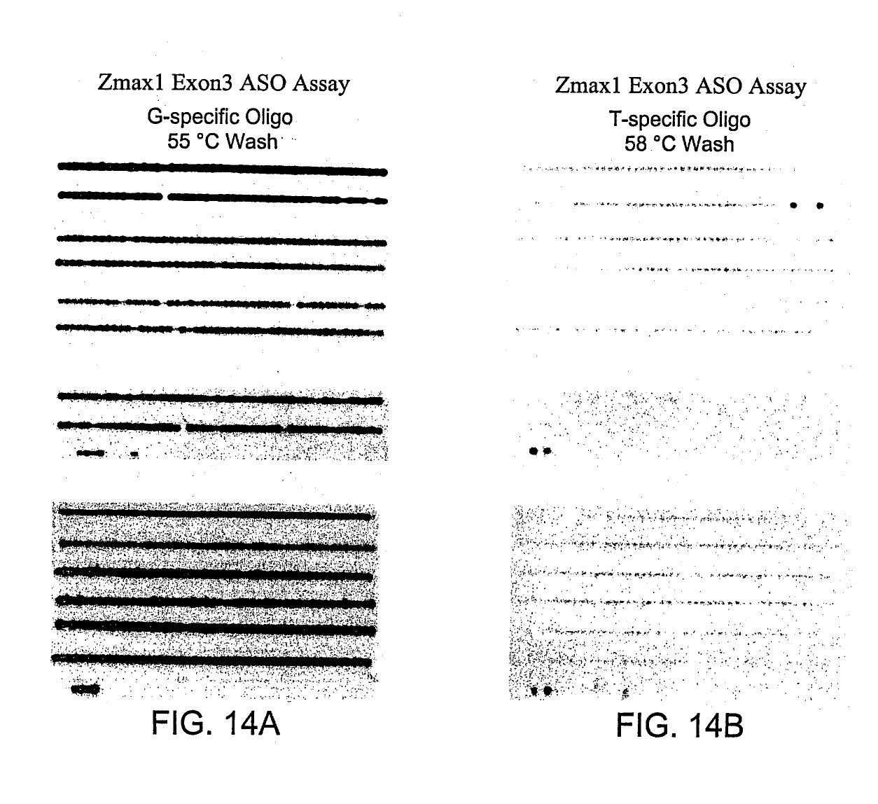

In order to determine the HBM1 allele frequency in ethnically diverse populations, 672 random individuals from various ethnic groups were typed by the allele specific oligonucleotide (ASO) method. This population included 96 CEPH grandparents (primarily Caucasian), 192 Caucasian, 192 African-American, 96 Hispanic, and 96 Asian individuals. No evidence was obtained for the presence of the HBM1 polymorphism in any of these individuals. Overall, a total of 911 individuals were typed either by direct sequencing or ASO hybridization; all were homozygous GG at the site of the HBM polymorphism (FIG. 14). This information illustrates that the HBM1 allele is rare in various ethnic populations.

Thus this invention provides a rapid method of identifying individuals with the HBM1 allele. This method could be used in the area of diagnostics and screening of an individual for susceptibility to osteoporosis or other bone disorder. The assay could also be used to identify additional individuals with the HBM1 allele or the additional polymorphisms described herein.

XIII. Cellular Localization of Zmax1

A. Gene Expression in Rat Tibia by non Isotopic In Situ Hybridization

In situ hybridization was conducted by Pathology Associates International (PAI), Frederick, Md. This study was undertaken to determine the specific cell types that express the Zmax1 gene in rat bone with particular emphasis on areas of bone growth and remodeling. Zmax1 probes used in this study were generated from both human (HuZmax1) and mouse (MsZmax1) cDNAs, which share an 87% sequence identity. The homology of human and mouse Zmax1 with rat Zmax1 is unknown.

For example, gene expression by non-isotopic in situ hybridization was performed as follows, but other methods would be known to the skilled artisan. Tibias were collected from two 6 to 8 week old female Sprague Dawley rats euthanized by carbon dioxide asphyxiation. Distal ends were removed and proximal tibias were snap frozen in OCT embedding medium with liquid nitrogen immediately following death. Tissues were stored in a −80° C. freezer.

Probes for amplifying PCR products from cDNA were prepared as follows. The primers to amplify PCR products from a cDNA clone were chosen using published sequences of both human LRP5 (Genbank Accession No. ABO17498) and mouse LRP5 (Genbank Accession No. AFO64984). In order to minimize cross reactivity with other genes in the LDL receptor family, the PCR products were derived from an intracellular portion of the protein coding region. PCR was performed in a 50 μI reaction volume using cDNA clone as template. PCR reactions contained 1.5 mM MgCl₂, 1 unit Amplitaq, 200 μM dNTPs and 2 μM each primer. PCR cycling conditions were 94° C. for 1 min., followed by 35 cycles of 94° C. for 30 seconds, 55° C. for 30 seconds, 72° C. for 30 seconds; followed by a 5 minute extension at 72° C. The reactions were then run on a 1.5% agarose Tris-Acetate gel. DNA was eluted from the agarose, ethanol precipitated and resuspended in 10 mM Tris, pH 8.0. Gel purified PCR products were prepared for both mouse and human cDNAs and supplied to Pathology Associates International for in situ hybridizations.

The sequence of the human and mouse PCR primers and products were as follows:

Human Zmax1 sense primer (HBM1253)
CCCGTGTGCTCCGCCGCCCAGTTC
Human Zmax1 antisense primer (HBM1465)
GGCTCACGGAGCTCATCATGGACTT
Human Zmax1 PCR product
CCCGTGTGCTCCGCCGCCCAGTTCCCCTGCGCGCGGGGTCAGTGTGTGGA
CCTGCGCCTGCGCTGCGACGGCGAGGCAGACTGTCAGGACCGCTCAGACG
AGGTGGACTGTGACGCCATCTGCCTGCCCAACCAGTTCCGGTGTGCGAGC
GGCCAGTGTGTCCTCATCAAACAGCAGTGCGACTCCTTCCCCGACTGTAT
CGACGGCTCCGACGAGCTCATGTGTGAAATCACCAAGCCGCCCTCAGACG
ACAGCCCGGCCCACAGCAGTGCCATCGGGCCCGTCATTGGCATCATCCTC
TCTCTCTTCGTCATGGGTGGTGTCTATTTTGTGTGCCAGCGCGTGGTGTG
CCAGCGCTATGCGGGGGCCAACGGGCCCTTCCCGCACGAGTATGTCAGCG
GGACCCCGCACGTGCCCCTCAATTTCATAGCCCCGGGCGGTTCCCAGCAT
GGCCCCTTCACAGGCATCGCATGCGGAAAGTCCATGATGAGCTCCGTGAG
CC
Mouse Zmax1 Sense primer (HBM1655)
AGCGAGGCCACCATCCACAGG
Mouse Zmax1 antisense primer (HBM1656)
TCGCTGGTCGGCATAATCAAT
Mouse Zmax1 PCR product
AGCAGAGCCACCATCCACAGGATCTCCCTGGAGACTAACAACAACGATGT
GGCTATCCCACTCACGGGTGTCAAAGAGGCCTCTGCACTGGACTTTGATG
TGTCCAACAATCACATCTACTGGACTGATGTTAGCCTCAAGACGATCAGC
CGAGCCTTCATGAATGGGAGCTCAGTGGAGCACGTGATTGAGTTTGGCCT
CGACTACCCTGAAGGAATGGCTGTGGACTGGATGGGCAAGAACCTCTATT
GGGCGGACACAGGGACCAACAGGATTGAGGTGGCCCGGCTGGATGGGCAG
TTCCGGCAGGTGCTTGTGTGGAGAGACCTTGACAACCCCAGGTCTCTGGC
TCTGGATCCTACTAAAGGCTACATCTACTGGACTGAGTGGGGTGGCAAGC
CAAGGATTGTGCGGGCCTTCATGGATGGGACCAATTGTATGACACTGGTA
GACAAGGTGGGCCGGGCCAACGACCTCACCATTGATTATGCCGACCAGCG
A

Riboprobes were synthesized as follows. The PCR products were reamplified with chimeric primers designed to incorporate either a T3 promoter upstream, or a T7 promoter downstream of the reamplification products. The resulting PCR products were used as template to synthesize digoxigenin-labeled riboprobes by in vitro transcription (IVT). Antisense and sense riboprobes were synthesized using T7 and T3 RNA polymerases, respectively, in the presence of digoxigenin-1 I-UTP (Boehringer-Mannheim) using a MAXIscript IVT kit (Ambion) according to the manufacturer. The DNA was then degraded with Dnase-1, and unincorporated digoxigenin was removed by ultrafiltration. Riboprobe integrity was assessed by electrophoresis through a denaturing polyacrylamide gel. Molecular size was compared with the electrophoretic mobility of a 100-1000 base pair (bp) RNA ladder (Ambion). Probe yield and labeling was evaluated by blot immunochemistry. Riboprobes were stored in 5 μl aliquots at −80° C.

The in situ hybridization was performed as follows. Frozen rat bone was cut into 5 μM sections on a Jung CM3000 cryostat (Leica) and mounted on adhesive slides (Instrumedics). Sections were kept in the cryostat at −20° C. until all the slides were prepared in order to prevent mRNA degradation prior to post-fixation for 15 minutes in 4% paraformaldehyde. Following post-fixation, sections were incubated with 1 ng/μl of either antisense or sense riboprobe in Pathology Associates International (PAI) customized hybridization buffer for approximately 40 hours at 58° C. Following hybridization, slides were subjected to a series of post-hybridization stringency washes to reduce nonspecific probe binding. Hybridization was visualized by immunohistochemistry with an anti-digoxigenin antibody (FAB fragment) conjugated to alkaline phosphatase. Nitroblue tetrazolium chloride/bromochloroindolyl phosphate (Boehringer-Mannheim), a precipitating alkaline phosphatase substrate, was used as the chromogen to stain hybridizing cells purple to nearly black, depending on the degree of staining. Tissue sections were counter-stained with nuclear fast red. Assay controls included omission of the probe, omission of probe and anti-digoxigenin antibody.

Specific cell types were assessed for demonstration of hybridization with antisense probes by visualizing a purple to black cytoplasmic and/or peri-nuclear staining indicating a positive hybridization signal for mRNA. Each cell type was compared to the replicate sections, which were hybridized with the respective sense probe. Results were considered positive if staining was observed with the antisense probe and no staining or weak background with the sense probe.

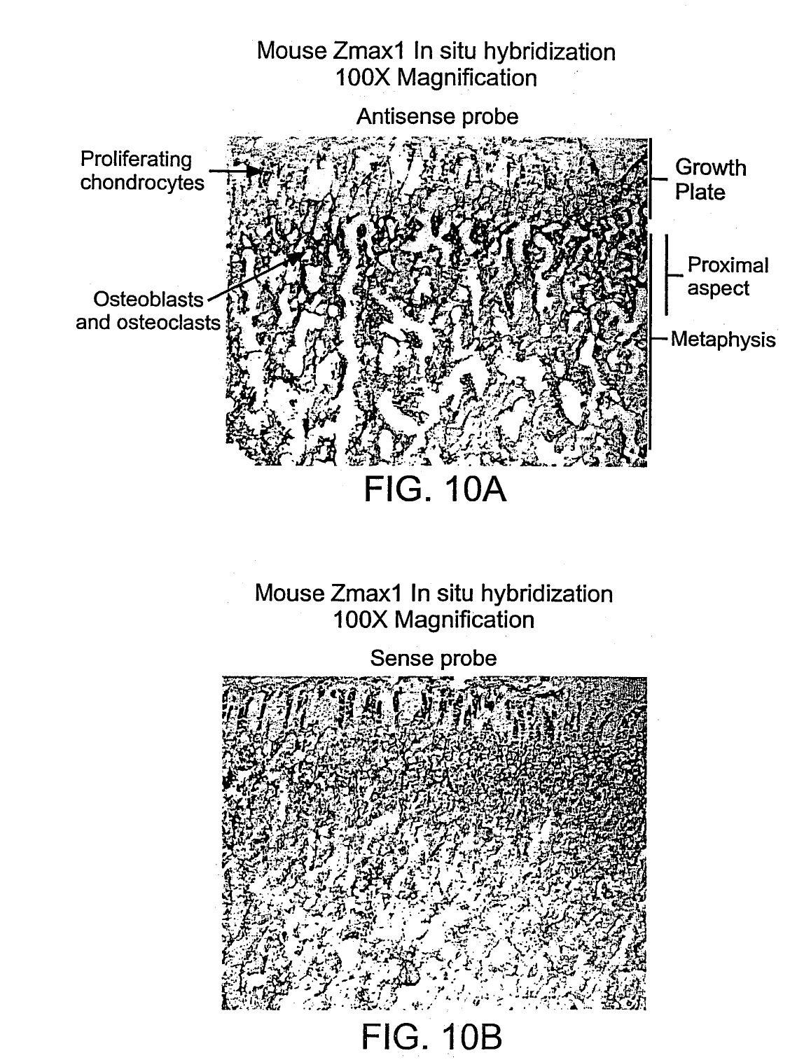

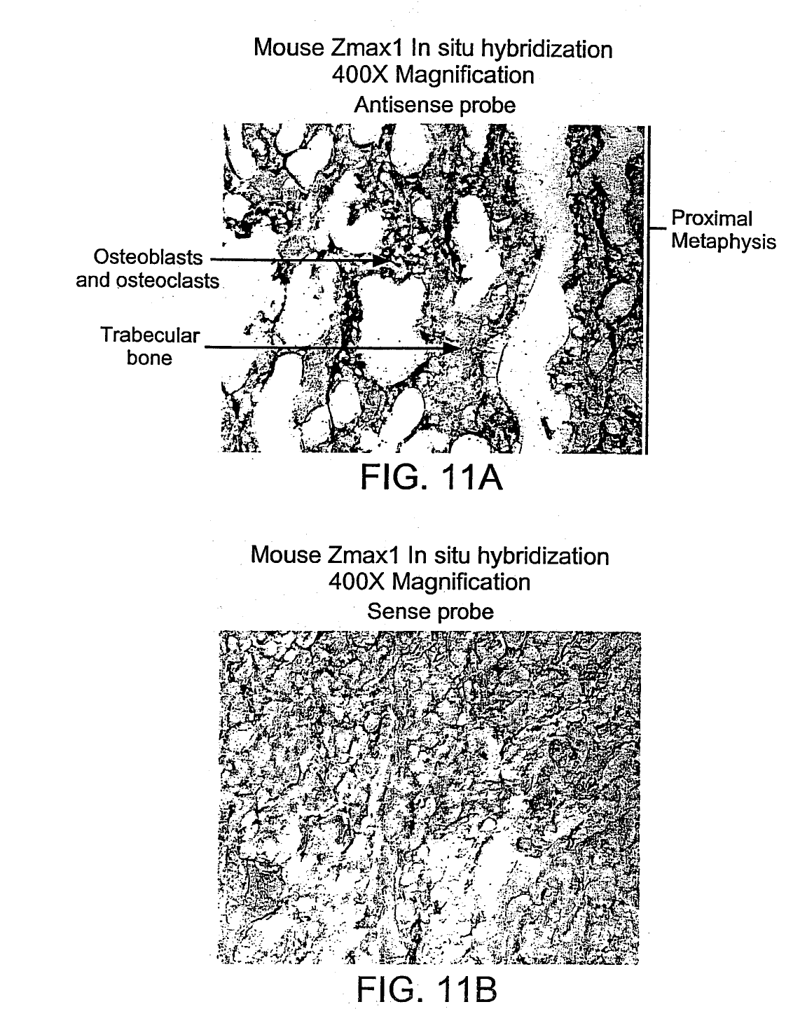

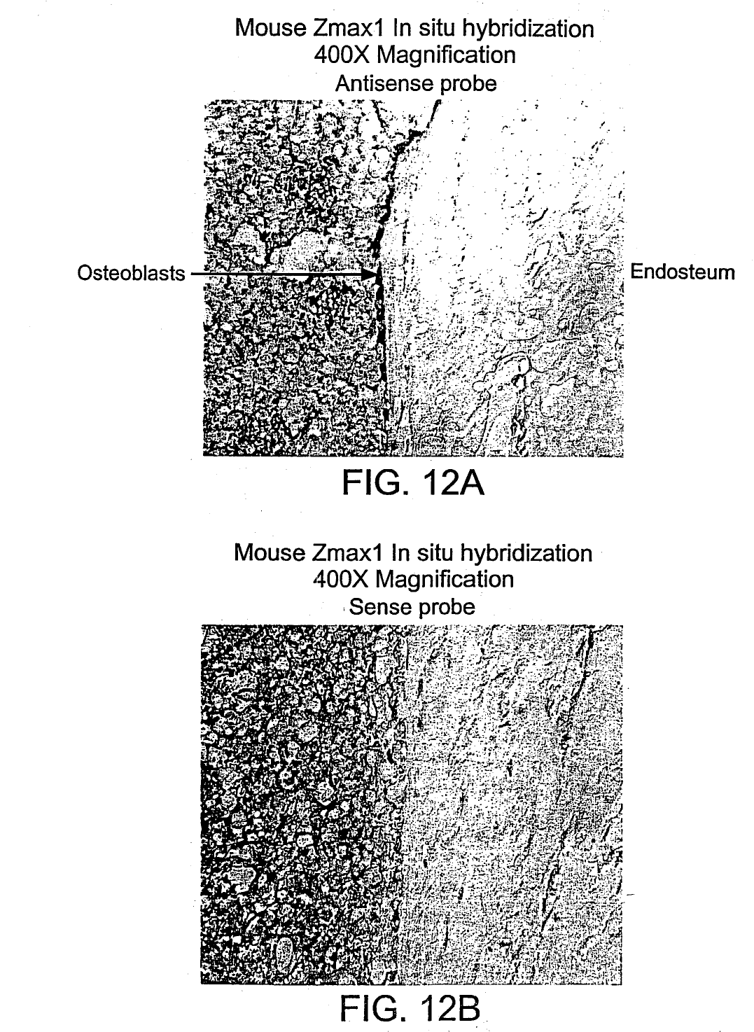

The cellular localization of the hybridization signal for each of the study probes is summarized in Table 5. Hybridization for Zmax1 was primarily detected in areas of bone involved in remodeling, including the endosteum and trabecular bone within the metaphysis. Hybridization in selected bone lining cells of the periosteum and epiphysis were also observed. Positive signal was also noted in chondrocytes within the growth plate, particularly in the proliferating chondrocytes. See FIGS. 10, 11 and 12 for representative photormicrographs of in situ hybridization results.

TABLE 5
Summary of Zmax1 in situ hybridization in rat tibia

| PROBE | SITE | ISH SIGNAL |
|---|---|---|
| Hu Zmax1 | Epiphysis | |
| | Osteoblasts | + |
| | Osteoclasts | − |
| | Growth Plate | |
| | resting chondrocytes | − |
| | proliferating chondrocytes | + |
| | hypertrophic chondrocytes | − |
| | Metaphysis | |
| | osteoblasts | + |
| | osteoclasts | + |
| | Diaphysis | − |
| | Endosteum | |
| | osteoblasts | + |
| | osteoclasts | + |
| | Periosteum | − |
| MsZmax1 | Epiphysis | |
| | Osteoblasts | + |
| | Osteoclasts | − |
| | Growth Plate | |
| | resting chondrocytes | − |
| | proliferating chondrocytes | + |
| | hypertrophic chondrocytes | + |
| | Metaphysis | |
| | osteoblasts | + |
| | osteoclasts | + |
| | Diaphysis | − |
| | Endosteum | |
| | osteoblasts | + |
| | osteoclasts | + |
| | Periosteum | + |

Legend: “+” = hybridization signal detected “−” = no hybridization signal detected
“ISH” - In situ hybridization

These studies confirm the positional expression of Zmax1 in cells involved in bone remodeling and bone formation. Zmax1 expression in the zone of proliferation and in the osteoblasts and osteoclasts of the proximal metaphysis, suggests that the Zmax1 gene is involved in the process of bone growth and mineralization. The activity and differentiation of osteoblasts and osteoclasts are closely coordinated during development as bone is formed and during growth as well as in adult life as bone undergoes continuous remodeling. The formation of internal bone structures and bone remodeling result from the coupling of bone resorption by activated osteoclasts with subsequent deposition of new material by osteoblasts. Zmax1 is related to the LDL receptor gene, and thus may be a receptor involved in mechanosensation and subsequent signaling in the process of bone remodeling. Therefore, changes in the level of expression of this gene could impact on the rate of remodeling and degree of mineralization of bone.

XIV. Antisense

Antisense oligonucleotides are short synthetic nucleic acids that contain complementary base sequences to a targeted RNA. Hybridization of the RNA in living cells with the antisense oligonucleotide interferes with RNA function and ultimately blocks protein expression. Therefore, any gene for which the partial sequence is known can be targeted by an antisense oligonucleotide.

Antisense technology is becoming a widely used research tool and will play an increasingly important role in the validation and elucidation of therapeutic targets identified by genomic sequencing efforts.

Antisense technology was developed to inhibit gene expression by utilizing an oligonucleotide complementary to the mRNA that encodes the target gene. There are several possible mechanisms for the inhibitory effects of antisense oligonucleotides. Among them, degradation of mRNA by RNase H is considered to be the major mechanism of inhibition of protein function. This technique was originally used to elucidate the function of a target gene, but may also have therapeutic applications, provided it is designed carefully and properly.

An example of materials and methods for preparing antisense oligonucleotides can be performed as follows. Preliminary studies have been undertaken in collaboration with Sequiter (Natick, Mass.) using the antisense technology in the osteoblast-like murine cell line, MC3T3. These cells can be triggered to develop along the bone differentiation sequence. An initial proliferation period is characterized by minimal expression of differentiation markers and initial synthesis of collagenous extracellular matrix. Collagen matrix synthesis is required for subsequent induction of differentiation markers. Once the matrix synthesis begins, osteoblast marker genes are activated in a clear temporal sequence: alkaline phosphatase is induced at early times while bone sialoprotien and osteocalcin appear later in the differentiation process. This temporal sequence of gene expression is useful in monitoring the maturation and mineralization process. Matrix mineralization, which does not begin until several days after maturation has started, involves deposition of mineral on and within collagen fibrils deep within the matrix near the cell layer-culture plate interface. The collagen fibril-associated mineral formed by cultured osteoblasts resembles that found in woven bone in vivo and therefore is used frequently as a study reagent.

MC3T3 cells were transfected with antisense oligonucleotides for the first week of the differentiation, according to the manufacturer's specifications (U.S. Pat. No. 5,849,902).

The oligonucleotides designed for Zmax1 are given below:

| | |
|---|---|
| 10875: | AGUACAGCUUCUUGCCAACCCAGUC |
| 10876: | UCCUCCAGGUCGAUGGUCAGCCCAU |
| 10877: | GUCUGAGUCCGAGUUCAAAUCCAGG |

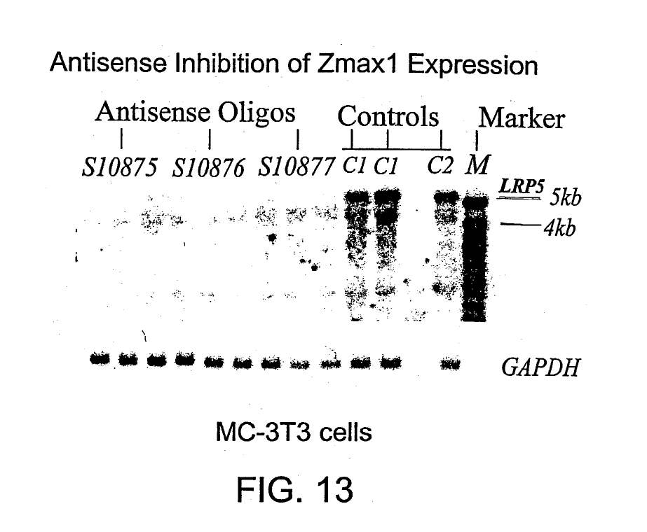

FIG. 13 shows the results of antisense inhibition of Zmax1 in MC3T3 cells. The three oligonucleotides shown above were transfected into MC3T3 and RNA was isolated according to standard procedures. Northern analysis clearly shows markedly lower steady state levels of the Zmax1 transcript while the control gene GAPDH remained unchanged. Thus, antisense technology using the primers described above allows for the study of the role of Zmax1 expression on bone biology.

XV. Yeast Two Hybrid

In order to identify the signaling pathway that Zmax1 participates in to modulate bone density, the yeast two hybrid protein interaction technology was utilized. This technique facilitates the identification of proteins that interact with one another by coupling tester proteins to components of a yeast transcription system (Fields and Song, 1989, Nature 340: 245-246; U.S. Pat. No. 5,283,173 by Fields and Song; Johnston, 1987, Microbiol. Rev. 51: 458-476; Keegan et al, 1986, Science 231: 699-704; Durfee et al, 1993, Genes Dev. 7: 555-569; Chien et al, 1991, Proc. Natl. Acad. Sci USA 88: 9578-9582; Fields et al., 1994, Trends in Genetics 10: 286-292; and Gyuris et al., 1993, Cell 75: 791-803). First a “bait” protein, the protein for which one seeks interacting proteins, is fused to the DNA binding domain of a yeast transcription factor. Second, a cDNA library is constructed that contains cDNAs fused to the transcriptional activation domain of the same yeast transcription factor; this is termed the prey library. The bait construct and prey library are transformed into yeast cells and then mated to produce diploid cells. If the bait interacts with a specific prey from the cDNA library, the activation domain is brought into the vicinity of the promoter via this interaction. Transcription is then driven through selectable marker genes and growth on selective media indicates the presence of interacting proteins.

The amino acid sequence used in the yeast two hybrid experiments discussed herein consisted of the entire cytoplasmic domain and a portion of the transmembrane domain and is shown below (amino to carboxy orientation):

RVVCQRYAGA NGPFPHEYVS GTPHVPLNFI APGGSQHGPF TGIACGKSMM
SSVSLMGGRG GVPLYDRNHV TGASSSSSSS TKATLYPPIL NPPPSPATDP
SLYNMDMFYS SNIPATVRPY RPYIIRGMAP PTTPCSTDVC DSDYSASRWK
ASKYYLDLNS DSDPYPPPPT PHSQYLSAED SCPPSPATER SYFHLFPPPP
SPCTDSS

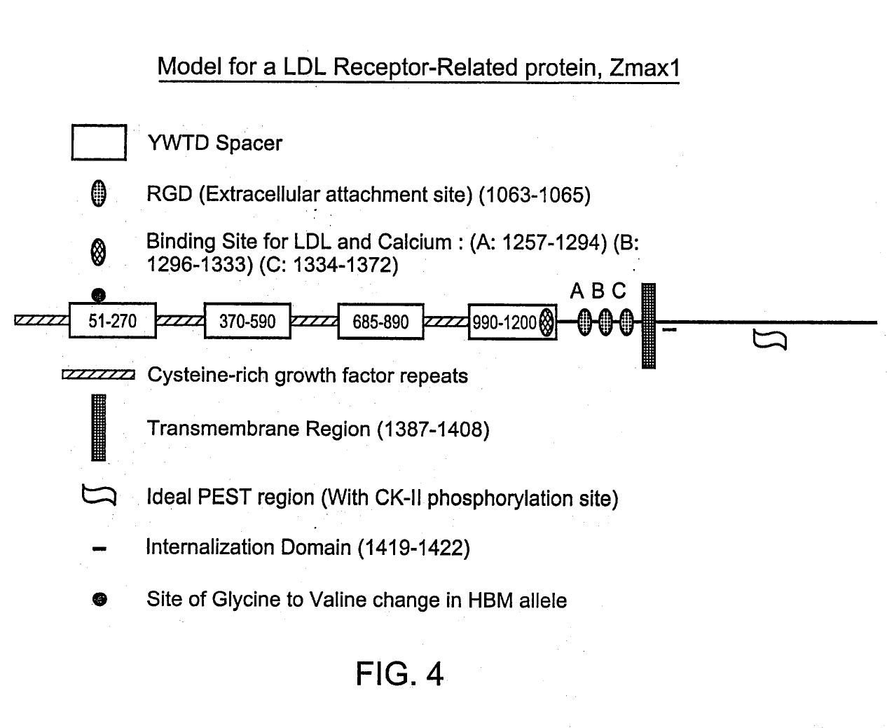

The last 6 amino acids of the putative transmembrane domain are indicated in bold. Putative SH3 domains are underlined. Additional amino acid sequences of 50 amino acids or greater in either the proteins encoded by the Zmax1 or HBM alleles can also be used as bait. The upper size of the polypeptide used as bait is limited only by the presence of a complete transmembrane domain (see FIG. 4), which will render the bait to be nonfunctional in a yeast two hybrid system. These additional bait proteins can be used to identify additional proteins which interact with the proteins encoded by HBM or Zmax1 in the focal adhesion signaling pathway or in other pathways in which these HBM or Zmax1 proteins may act. Once identified, methods of identifying agents which regulate the proteins in the focal adhesion signaling pathway or other pathways in which HBM acts can be performed as described herein for the HBM and Zmax1 proteins.

In order to identify cytoplasmic Zmax1 signaling pathways, the cytoplasmic domain of Zmax1 was subcloned into two bait vectors. The first vector was pDBleu, which was used to screen a brain, and Hela prey cDNA library cloned into the vector pPC86 (Clontech). The second bait vector used was pDBtrp, which was used to screen a cDNA prey library derived from the TE85 osteosarcoma cell line in vector pOP46. Standard techniques known to those skilled in the art were used as described in Fields and Song, 1989, Nature 340: 245-246; U.S. Pat. No. 5,283,173 by Fields and Song; Johnston, 1987, Microbiol. Rev. 51: 458-476; Keegan et al., 1986, Science 231: 699-704; Durfee et al., 1993, Genes Dev. 7: 555-569; Chien et al., 1991, Proc. Natl. Acad. Sci USA 88: 9578-9582; Fields et al., 1994, Trends in Genetics 10: 286-292; and Gyuris et al., 1993, Cell 75: 791-803. The bait construct and prey cDNA libraries were transformed into yeast using standard procedures.

To perform the protein interaction screen, an overnight culture of the bait yeast strain was grown in 20 ml SD selective medium with 2% glucose (pDBLeu, SD-Leu medium, pDBtrp, SD-trp medium). The cultures were shaken vigorously at 30° C. overnight. The cultures were diluted 1: 10 with complete medium (YEPD with 2% glucose) and the cultures then incubated with shaking for 2 hrs at 30° C.

The frozen prey library was thawed, and the yeast cells reactivated by growing them in 150 ml YEPD medium with 2% glucose for 2 hrs at 30° C. A filter unit was sterilized with 70% ethanol and washed with sterile water to remove the ethanol. The cell densities of both bait and prey cultures were measured by determining the OD at 600 um. An appropriate volume of yeast cells that corresponded to a cell number of 1 ml of OD 600=4 of each yeast strain, bait and prey (library) was placed in a 50 ml Falcon tube. The mixture was then filtered through the sterilized filter unit. The filter was then transferred onto a prewarmed YEPD agar plate with the cell side up, removing all air bubbles underneath the filter. Plates were then incubated at 30° C. for 6 hrs. One filter was transferred into a 50 ml Falcon tube, and 10 ml of SD with 2% Glucose was added; cells were resuspended by vortexing for 10 sec.

The number of primary diploid cells (growth on SD-Leu, -Trp plates) versus the numbers of colony forming units growing on SD-Trp and SD-Leu plates only was then titered. Different dilutions were plated and incubated at 30° C. for two days. The number of colony forming units was then counted. The number of diploid colonies (colonies on SD-Leu-Trp plates) permits the calculation of whether or not the whole library of prey constructs was mated to the yeast expressing the bait. This information is important to judge the quality of the screen.

A. Indirect Selection

Resuspended cells from 5 filtermatings were then pooled and the cells sedimented by centrifugation in a 50 ml Falcon tube. Cells were then resuspended in 16 ml SD medium with 2% Glc. Two ml of this cell suspension was plated onto 8 square plates each (SD-Leu, -Trp) with sterile glass beads and selected for diploid cells by incubating at 30° C. for 18-20 hrs.

Cells were then scraped off the square plates, the cells centrifuged and combined into one 50 ml Falcon tube. The cell pellet was then resuspended in 25 ml of SD medium with 2% glucose. The cell number was then determined by counting of an appropriate dilution (usually 1:100 to 1:1000) with a Neugebauer chamber. Approximately 5×10⁷ diploid cells were plated onto the selective medium. The observations about the growth of the bait strain together with irrelevant prey vectors helps to determine which selective plates will have to be chosen for the library screen. Generally, all screens were plated on one square plate each with SD-Leu, -Trp, -His; SD-Leu, -Trp, His, 5 mM 3AT, and SD-Leu, -Trp, -His, -Ade is recommended.

The yeast cells were then spread homogeneously with sterile glass beads and incubated at 30° C. for 4 days. The number of colony forming yeast cells was titered by plating different dilutions of the scraped cell suspension onto SD-Leu, -Trp plates. Usually, plating of 100 μl of a 10⁻³ and 10⁻⁴ dilution gave 100-1000 colonies per plate.

B. Direct Selection

Five filters with the mated yeast cells were each transferred into separate 50 ml Falcon tubes and the cells resuspended with 10 ml SD medium with 2% Glc, each, followed by vortexing for 10 sec. The resuspended cells were combined and centrifuged in a Beckman centrifuge at 3000 rpm. The supernatant was discarded and the cells resuspended in 6 ml of SD medium with 2% Glc. Two ml of the suspension was spread onto each selective square plate and incubated at 30° C. for 4-5 days.

C. Isolation of Single Colonies

Yeast cells from an isolated colony were picked with a sterile tooth pick and transferred into individual wells of a 96 well plate. The cells were resuspended in 50 μl of SD-Leu, -Trp, -His medium and incubated at 30° C. for one day. The yeast cells were then stamped onto a SD-Leu, -Trp, -His plate in 96 well format and incubated at 30° C. for 2 days. Yeast cells were also stamped onto a Nylon filter covering a YEPD plate and incubated at 30° C. for one day. The cells on the Nylon filter were used for the analysis of the β-Gal reporter activity.

Yeast colonies were scraped from the SD-Leu, -Trp, -His plate with a sterile tooth pick, and reconfigured, if necessary, according to the β-Gal activity and then resuspended in 20% glycerol. This served as a master plate for storage at −80° C.

For DNA preparation, yeast cells from the glycerol stock were stamped onto a SD-Trp plate and incubated at 30° C. for 2 days. After two days of incubation, the yeast colonies were ready for colony PCR and sequencing. Standard colony PCR conditions were used to amplify inserts from preys recovered from the interaction screen. Sequencing was done using standard sequencing reactions and ABI377 (Perkin Elmer) fluorescent sequencing machines.

D. Verification of Bait/Prey Interaction

Glycerol stocks of the prey of interest were thawed and inoculated in a 10 ml overnight culture of SD with glucose-Trp. After overnight growth, plasmid DNA preparation was performed using the BIO 101 RPM Yeast Plasmid Isolation Kit with 10 ml of culture. The culture was centrifuged and transfered to a 1.5 ml microcentrifuge tube. Yeast Lysis Matrix was then added to the pellet followed by 250 μl of Alkaline Lysis Solution. Samples were then vortexed for 5 minutes. 250 μl Neutralizing Solution was added and the sample mixed briefly. Samples were centrifuiged for 2 minutes at room temperature in a microcentrifuge. The supernatant was transferred to a Spin Filter avoiding debris and Lysis Matrix. 250 μl of Glassmilk Spin Buffer was added, and the tubes inverted to mix. Samples were centrifuged for 1 min and the liquid in the Catch Tube was discarded. 500 μl of Wash Solution was added, the samples were centrifuged for 1 min, and the wash solution was discarded. The wash step was repeated once followed by a 1 min dry centrifugation to drive the remaining liquid out of the Spin Filter. The filter was transferred to a new Catch Tube and 100 μl of sterile H₂O was added; samples were then vortexed briefly to resuspend and centrifuged for 30 seconds to collect the DNA in the bottom of the Catch Tube.

Five μl of DNA was then transformed into DH10B Electromax cells using standard procedures and glycerol stocks prepared. Miniprep DNA was prepared using the Qiagen QIAprep Spin Miniprep Kit. DNA was finally eluted with 30 ill of Qiagen EB buffer. One μl of the plasmid DNA samples was then used to transform yeast cells using standard procedures. After 2 days of growth on SD-trp media, colonies were picked and patched onto fresh media. Similarly, bait colonies were patched onto SD-Leu media. Both were grown overnight at 30° C.

For mating, cells from bait and prey patches were spread together on YAPD media and incubated at 30° C. for 12 hr. This plate was then replicaplated onto an SD Agar-Leu-Trp plate and grown for 2 days at 30° C. To test the strength of interaction these plates were replicaplated onto SD Agar-Leu-Trp-His, SD Agar-Leu-Trp-His with 5 mM 3AT and 10 mM 3AT, SD Agar-Leu-Trp-His-Ade, and SD Agar-Leu-Trp-Ura media and grown for 2 days at 30° C.

E. Galacton Star β-Galactosidase Activity Assay

After streaking and replica plating positive interactors on selection plates, colonies were placed in a 96 well dish with 200 μl of SD-medium, leaving wells 1 and 96 blank. Ten microliters from the first 96 well dish was plated into another flat bottom 96 well dish containing 100 μl of SD-medium. Controls consisted of a negative control and a very weak positive control. The cell density was measured at OD₆₀₀ (a value of 1 corresponds to 1×10⁷ cells utilizing a 96 well spectrophotometer). The OD was usually between 0.03 and 0.10. Using microplates specifically for the luminometer, 50 μl of reaction mixture were pipetted into each well. Fifty microliters of culture were then added and mixed by pipetting up and down twice. The reaction was incubated for 30 minutes at room temperature followed by measurement of Relative Light Units using a luminometer.

Table 6 lists the genes identified in the yeast two hybrid screens from the 3 prey libraries tested. Two genes, zyxin and axin, were found to interact with the cytoplasmic domain of Zmax1 in all three screens. Three genes, alpha-actinin, TCB and S1-5 interacted in two of the three screens.

A variety of proteins found at sites of cell-cell and cell-matrix contact (focal contacts/adesion plaques) were shown to interact with the cytoplasmic domain of Zmax1. These include alpha-actinin, Trio, Pinch-like protein, and Zyxin. PINCH is a LIM domain-containing protein that is known to interact with integrin-linked kinase, an early signaler in integrin and growth factor signaling pathways. The finding of a closely related gene in the yeast two hybrid screen raises the possibility of a novel pathway linked to integrin signaling from extracellular matrix signals. Trio, also known to localize to focal adhesions, is thought to play a key role in coordinating cell-matrix interactions and cytoskeletal rearrangements involved in cell movement. Zyxin, another LIM domain-containing protein, is also localized to adhesion plaques and is thought to be involved in reorganization of the cytoskeleton when triggers are transmitted via integrin signaling pathways. Zyxin also interacts with alpha actinin, which we identified as interacting with Zmax1. Other LIM domain containing proteins identified include the human homologue of mouse ajuba, LIMD1, and a novel LIMD1-like protein.

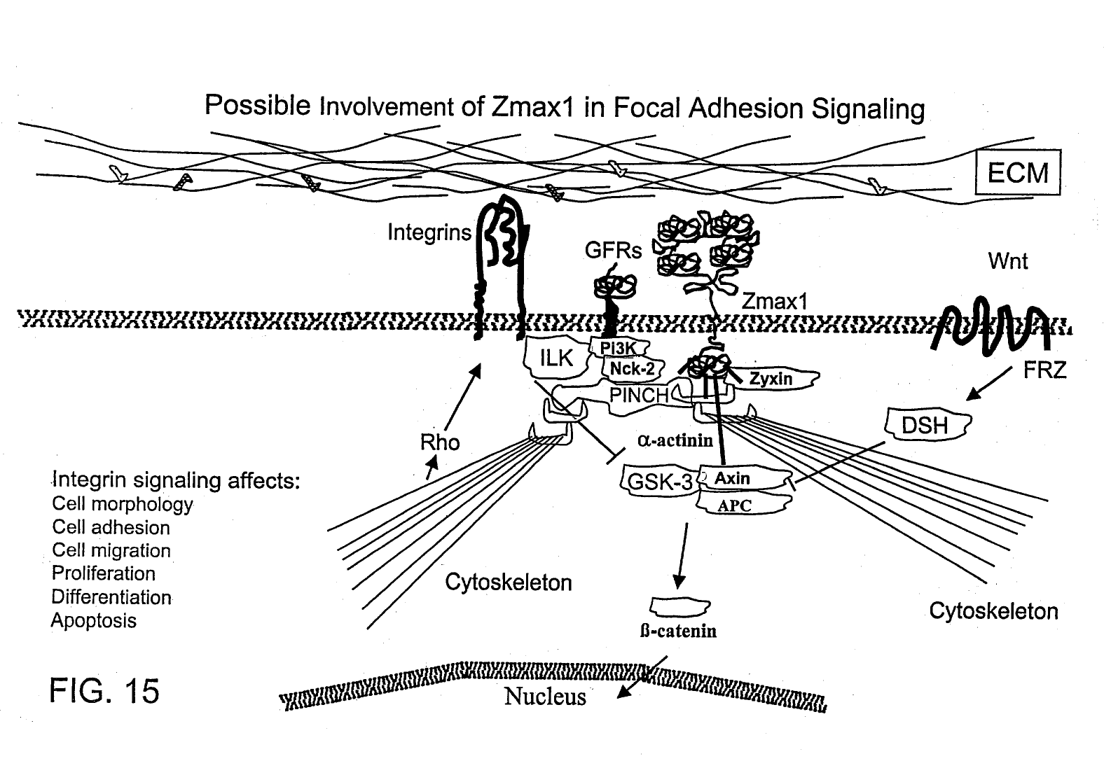

Axin was also identified from the two hybrid experiments. This protein is involved in inhibition of the Wnt signaling pathway and interacts with the tumor suppressor APC. There is a link here with the focal adhesion signaling described above: one common step in the two pathways involves inhibition of glycogen synthase kinase 3, which in turn results in the activation of β-catenin/Lef-1 and AP-1 transcription factors. Axin/APC are involved in this as well as integrin linked kinase. The Wnt pathway has a role in determining cell fates during embryogenesis. If inappropriately activated, the Wnt pathway may also lead to cancer. The Wnt pathway also seems to have a role in cytoskeletal rearrangements. A model depicting Zmax1 involvement in focal adhesion signaling is depicted in FIG. 15.

This data coupled with other studies suggest that integrin signaling pathways have a role in cellular responses to mechanical stress and adhesion. This provides an attractive model for the mechanism of action of Zmax1 in bone biology. It is possible that Zmax1 is involved in sensing either mechanical stress directly or binding a molecule in the extracellular matrix that is related to mechanical sensation. Signaling through subsequent pathways may be involved in bone remodeling due to effects on cell morphology, cell adhesion, migration, proliferation, differentiation, and apoptosis in bone cells.

TABLE 6

Yeast Two Hybrid Results

| Gene | | Genbank | NT | AA |
|---|---|---|---|---|
| Symbol | Gene | Accession # | SEQ ID NO: | SEQ ID NO: |
| ACTN1 | alpha-actinin | NM_001102 | 63 | |
| AES | amino-terminal enhancer of | NM_001130.3 | 64 | |
| AIP4 | atrophin-1 interacting protein | AF038564.1 | 65 | |
| Novel | Ajuba | | 66 | |
| AXIN | Wnt signaling | AF009674.1 | 67 | |
| CDC23 | cell division cycle 23, yeast, | NM_004661.1 | 68 | |
| | homolog | | | |
| HSM800944 | Similar to TRIO | AL117435.1 | 69 | |
| HSM800936 | | AL117427.1 | 70 | |
| Novel | Similar to LIM domains | | 71 | |
| | containing protein 1 | | | |
| DEEPEST | mitotic spindle coiled-coil | NM_006461.1 | 72 | |
| | related protein | | | |
| ECM1 | extracellular matrix protein 1 | U65932.1 | 73 | |
| EF1A | elongation factor 1-alpha | X16869.1 | 74 | |
| FN | fibronectin | X02761.1 | 75 | |
| HOXB13 | homeodomain protein | U81599.1 | 76 | |
| Novel | Glu-Lys Rich protein | | 77 | |
| LIMD1 | LIM domains containing 1 | NM_014240.1 | 78 | |
| Novel | PINCH-like | | 79 | |
| RANBPM | centrosomal protein | NM_005493.1 | 80 | |
| S1-5 | extracellular protein | U03877.1 | 81 | |
| TCB | gene encoding cytosolic | M26252.1 | 82 | |
| | thyroid hormone-binding | | | |
| TID | tumorous imaginal discs | NM_005147.1 | 83 | |
| ZYX | Zysin | NM_003461.1 | 84 | |
| TRIO | GTPase | U42390.1 | 85 | |
| HUMPITPB | phosphatidylinositol transfer | D30037.1 | 86 | |
| | protein | | | |
| ACTN1 | alpha-actinin | NP_001093.1 | | 87 |
| AES | amino-terminal enhancer of | NP_001121.2 | | 88 |
| AIP4 | atrophin-1 interacting protein | AAC04845.1 | | 89 |
| Novel | Ajuba | | | 90 |
| AXIN | Wnt signalling | AAC51624.1 | | 91 |
| CDC23 | cell division cycle 23, yeast | NP_004652.1 | | 92 |
| | homolog | | | |
| Novel | Similar to TRIO CAB55923.1 | | | 93 |
| Novel | Similar to LIM domains | | | 94 |
| | containing protein 1 | | | |
| DEEPEST | mitotic spindle coiled-coil | NP_006452.1 | | 95 |
| | related protein | | | |
| ECM1 | extracellular matrix protein 1 | AAB05933.1 | | 96 |
| EF1A | elongation factor 1-alpha | CAA34756.1 | | 97 |
| FN | fibronectin | CAA26536.1 | | 98 |
| Novel | Glu-Lys rich protein | | | 99 |
| HOXB13 | homeodomain protein B13 | AAB39863.1 | | 100 |
| LIMD1 | LIM domains containing 1 | NP_055055.1 | | 101 |
| Novel | PINCH-like | | | 102 |
| RANBPM | centrosomal protein | NP_005484.1 | | 103 |
| S1-5 | extracellular protein | AAA65590.1 | | 104 |
| TCB | cytosolic thyroid hormone-binding | AAA36672.1 | | 105 |
| | binding protein | | | |
| TID | tumorous imaginal discs | NP_005138.1 | | 106 |
| ZYX | Zyxin | NP_003452.1 | | 107 |
| TRIO | GTPase | AAC34245.1 | | 108 |
| PTDINSTP | phosphatidylinositol transfer | P48739 | | 109 |
| | protein beta isoform | | | |

In light of the model depicted in FIG. 15 and the results shown in Table 6, another aspect contemplated by the invention would be to regulate bone density and bone mass disorders by the regulating focal adhesion signaling. The regulation can occur by regulating the DNA, mRNA transcript or protein encoded by any of the members involved in the focal adhesion signaling pathway as identified by the yeast two hybrid system.

Also contemplated are the novel nucleic acids and proteins identified by the HBM yeast two hybrid system. These include but are not limited to SEQ ID NO: 66 (Ajuba), SEQ ID NO: 71 (a gene similar to a gene encoding LIM domains containing protein 1), SEQ ID NO: 77 (Glu-Lys Rich protein), SEQ ID NO: 79 (PINCH-like gene), SEQ ID NO: 90 (Ajuba protein), SEQ ID NO: 93 (protein similar to TRIO), SEQ ID NO: 94 ( ), SEQ ID NO: 99 (Glu-Lys rich protein) and SEQ ID NO: 102 (PINCH-like protein).

XVI. Potential Function

The protein encoded by Zmax1 is related to the Low Density Lipoprotein receptor (LDL receptor). See, Goldstein et al, Ann. Rev. Cell Biology, 1: 1-39 (1985); Brown et al, Science, 232:34-47 (1986). The LDL receptor is responsible for uptake of low density lipoprotein, a lipid-protein aggregate that includes cholesterol. Individuals with a defect in the LDL receptor are deficient in cholesterol removal and tend to develop artherosclerosis. In addition, cells with a defective LDL receptor show increased production of cholesterol, in part because of altered feedback regulation of cholesterol synthetic enzymes and in part because of increased transcription of the genes for these enzymes. In some cell types, cholesterol is a precursor for the formation of steroid hormones.

Thus, the LDL receptor may, directly or indirectly, function as a signal transduction protein and may regulate gene expression. Because Zmax1 is related to the LDL receptor, this protein may also be involved in signaling between cells in a way that affects bone remodeling.

The glycine 171 amino acid is likely to be important for the function of Zmax1 because this amino acid is also found in the mouse homologue of Zmax1. The closely related LRP6 protein also contains glycine at the corresponding position (Brown et al, Biochemical and Biophysical Research Comm., 248:879-888 (1988)). Amino acids that are important in a protein's structure or function tend to be conserved between species, because natural selection prevents mutations with altered amino acids at important positions from arising.

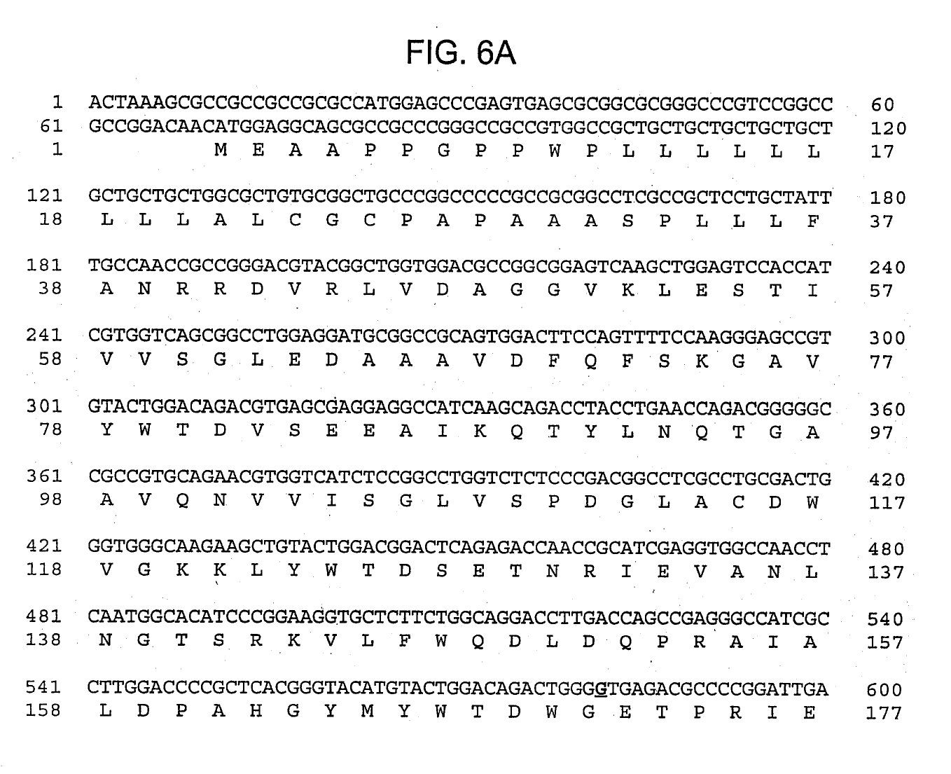

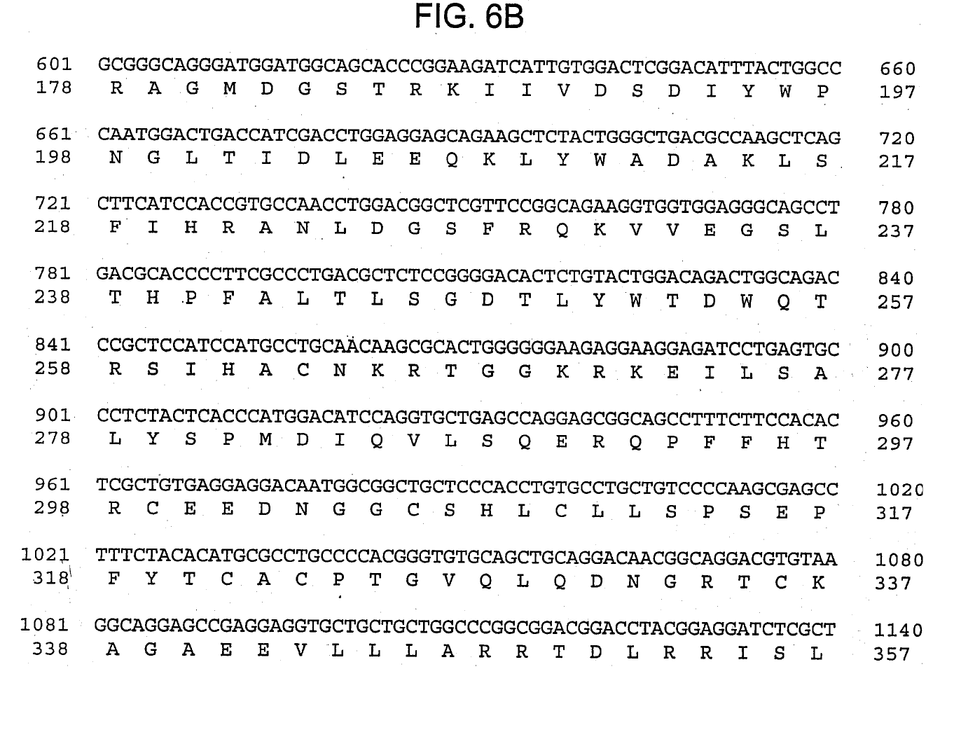

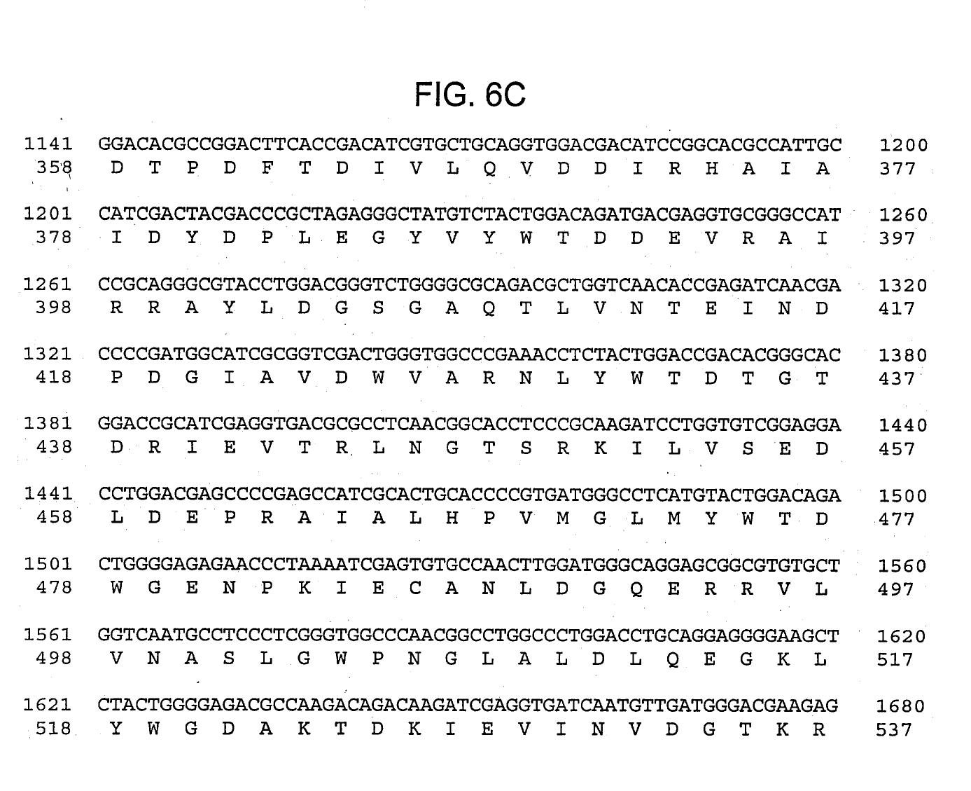

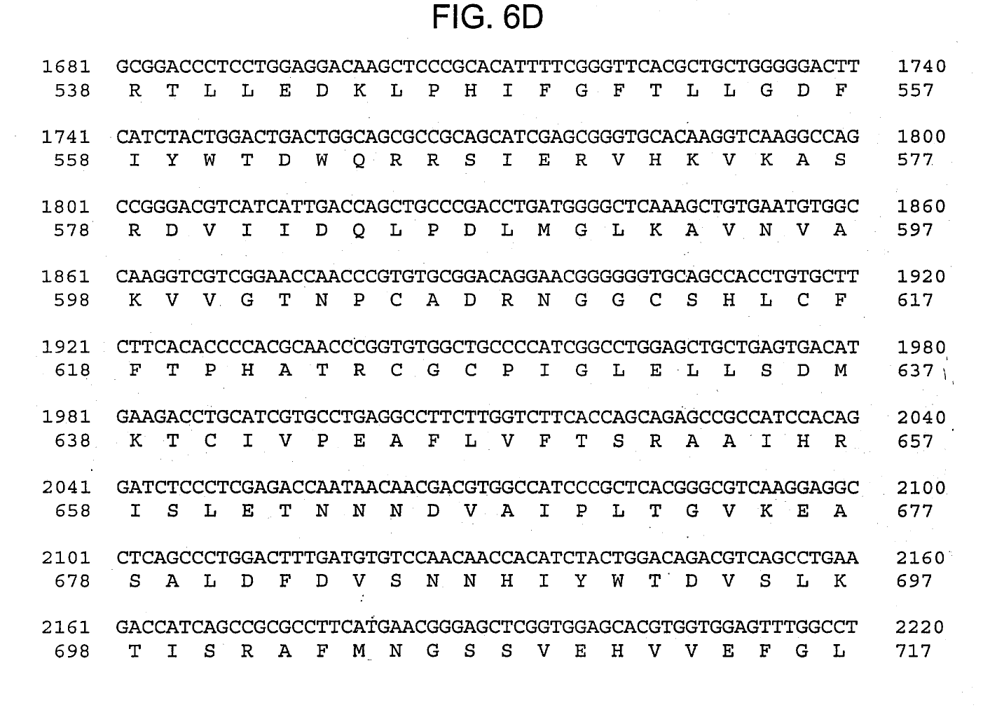

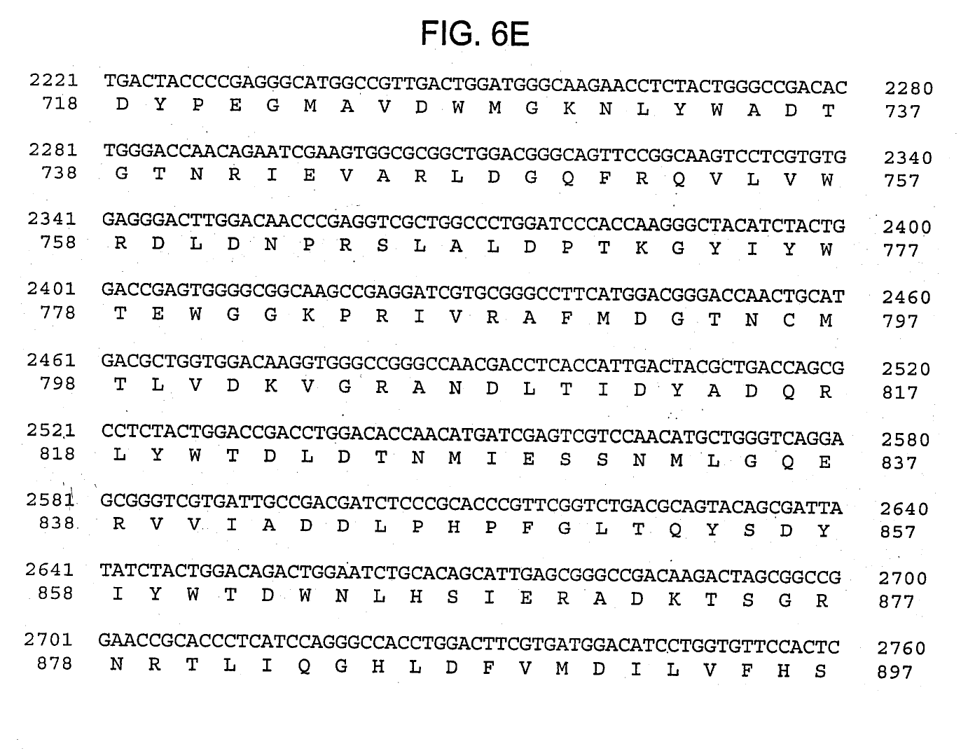

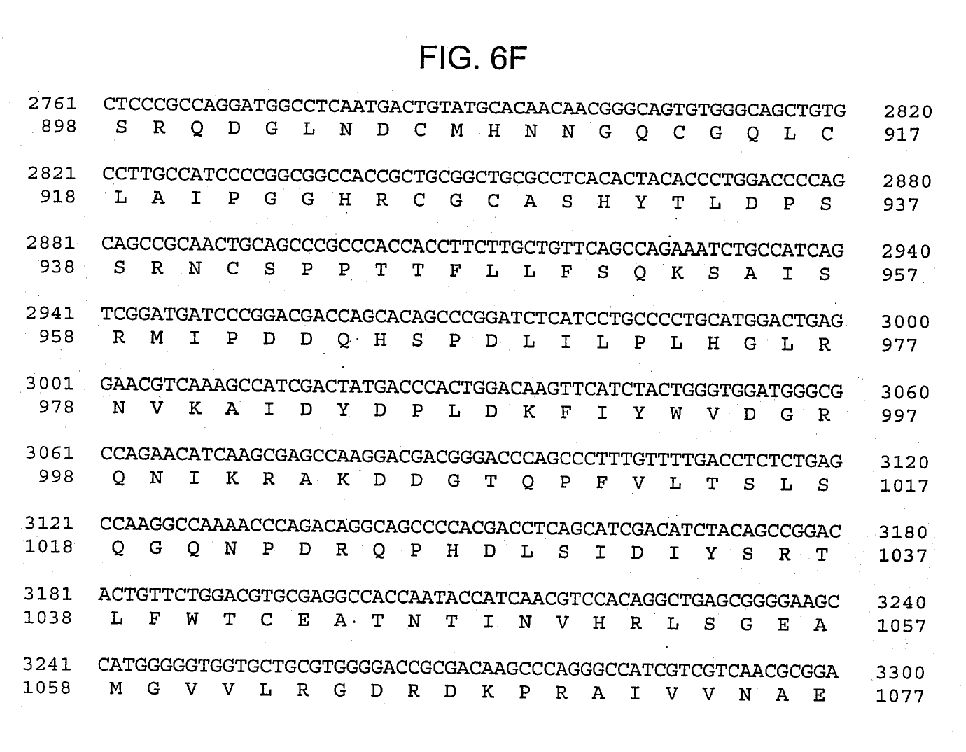

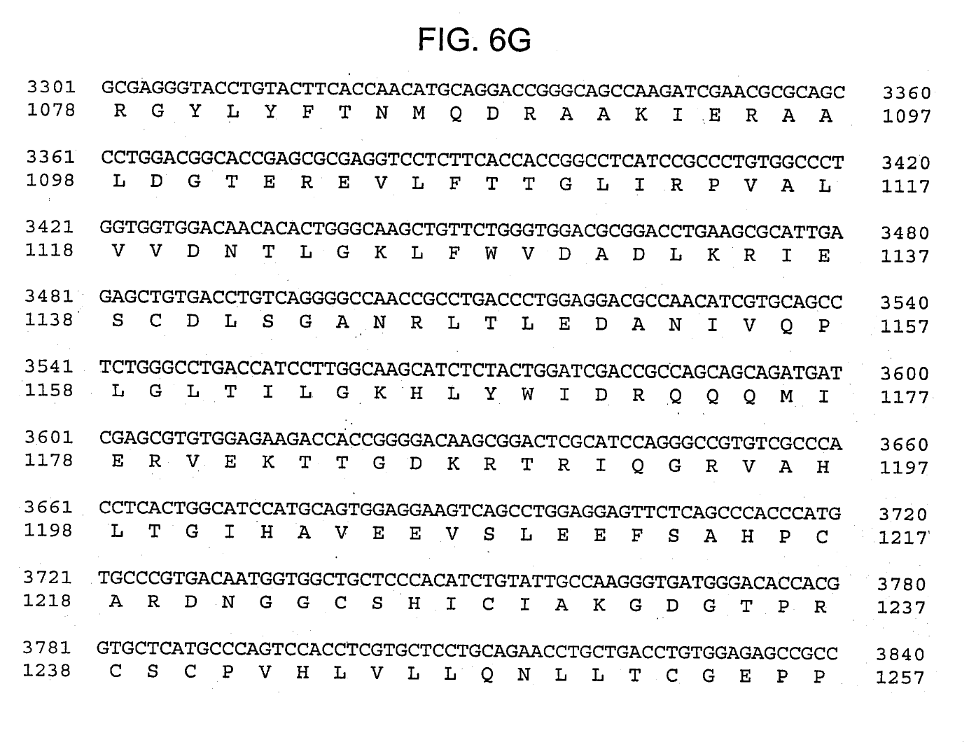

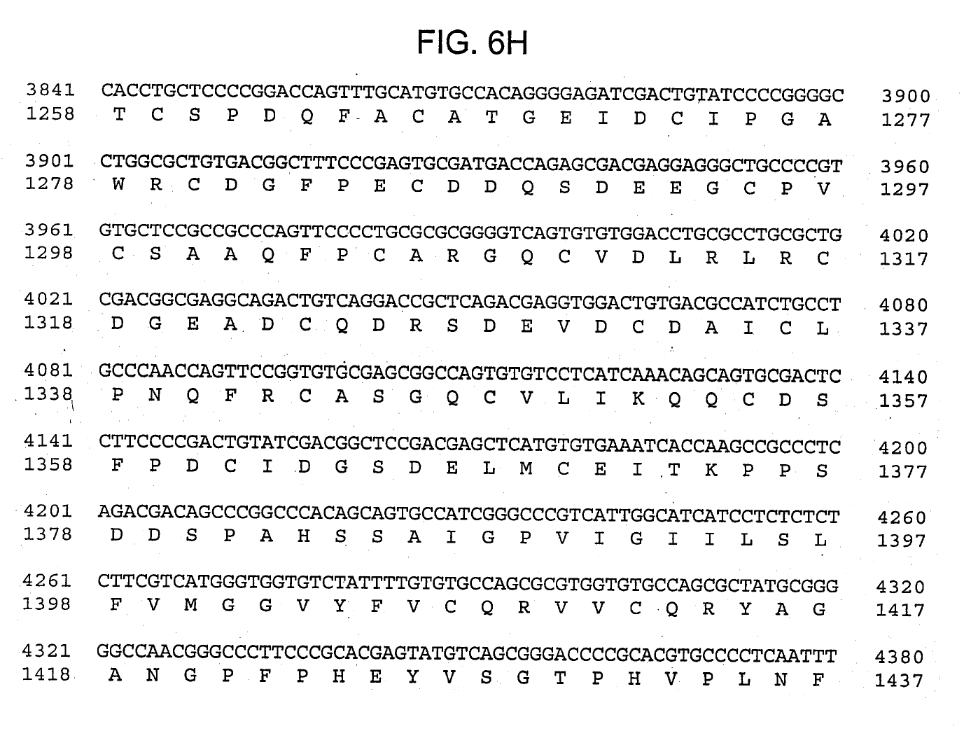

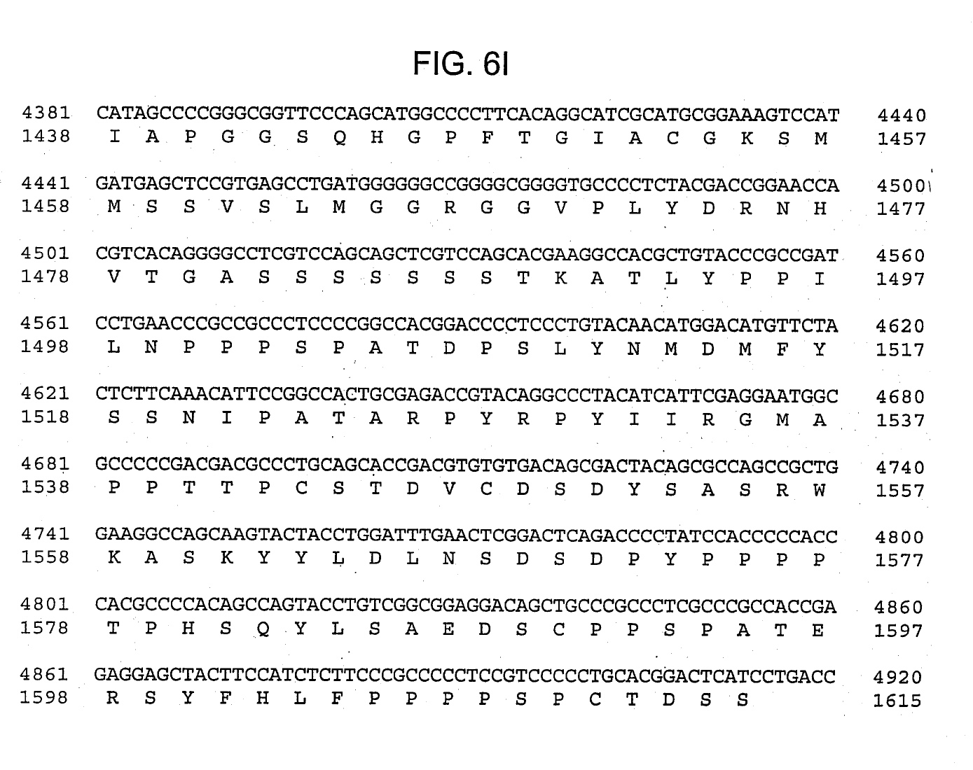

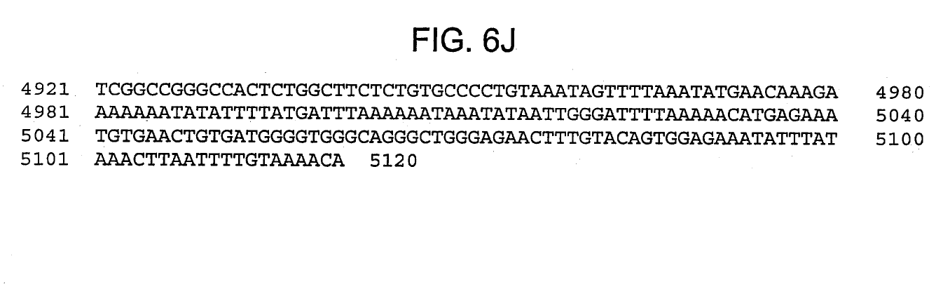

In addition, the extracellular domain of Zmax1 contains four repeats consisting of five YWTD motifs followed by an EFG motif. This 5YWTD+EGF repeat is likely to form a distinct folded protein domain, as this repeat is also found in the LDL receptor and other LDL receptor-related proteins. The first three 5YWTD+EGF repeats are very similar in their structure, while the fourth is highly divergent. Glycine 171 occurs in the central YWTD motif of the first 5YWTD+EGF repeat in Zmax1. The other two similar 5YWTD+EGF repeats of Zmax1 also contain glycine at the corresponding position, as does the 5YWTD+EGF repeat in the LDL receptor protein. However, only 17.6% of the amino acids are identical among the first three 5YWTD+EGF repeats in Zmax1 and the single repeat in the LDL receptor. These observations indicate that glycine 171 is essential to the function of this repeat, and mutation of glycine 171 causes a functional alteration of Zmax1. The cDNA and peptide sequences are shown in FIGS. 6A-6E. The critical base at nucleotide position 582 is indicated in bold and is underlined.

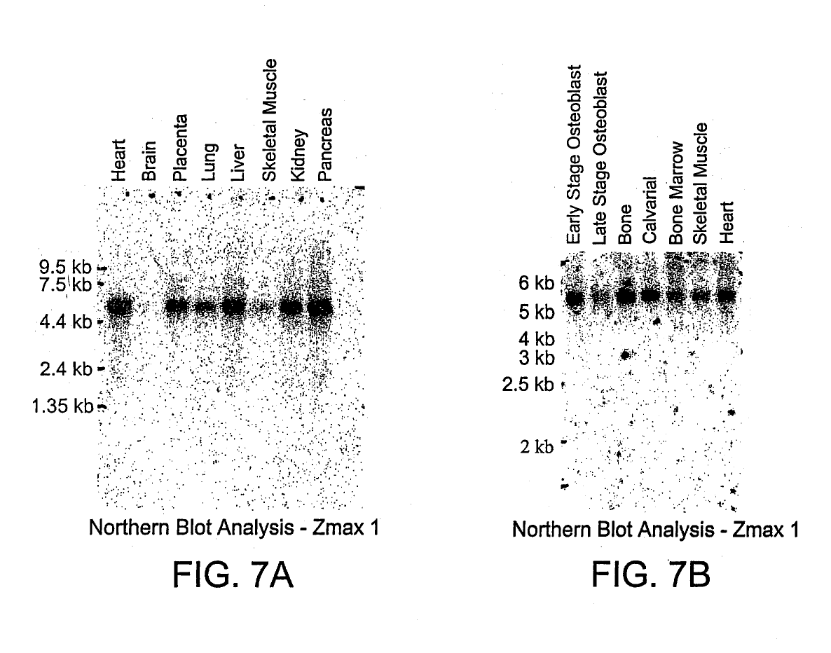

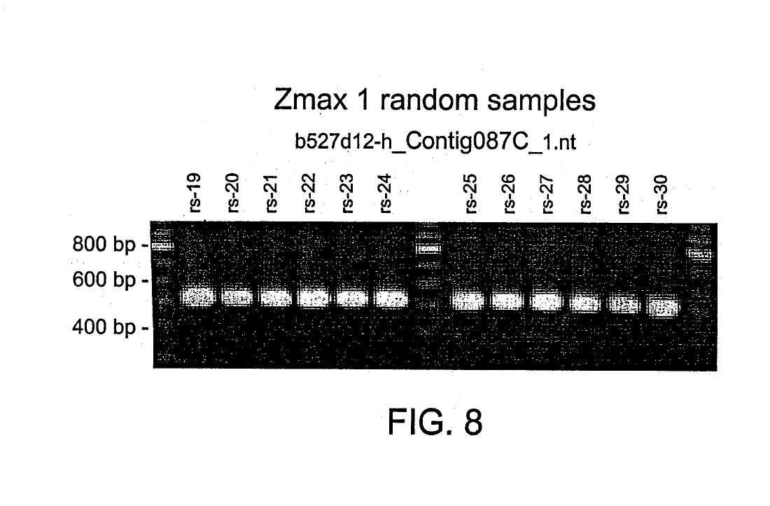

Northern blot analysis (FIGS. 7A-B) reveals that Zmax1 is expressed in human bone tissue as well as numerous other tissues. A multiple-tissue Northern blot (Clontech, Palo Alto, Calif.) was probed with exons from Zmax1. As shown in FIG. 7A, the 5.5 kb Zmax1 transcript was highly expressed in heart, kidney, lung, liver and pancreas and is expressed at lower levels in skeletal muscle and brain. A second northern blot, shown in FIG. 7B, confirmed the transcript size at 5.5 kb, and indicated that Zmax1 is expressed in bone, bone marrow, calvaria and human osteoblastic cell lines.

Taken together, these results coupled with the yeast two hybrid results indicate that the HBM polymorphism in the Zmax1 gene is responsible for the HBM phenotype, and that the Zmax1 gene is important in bone development. In addition, because mutation of Zmax1 can alter bone mineralization and development, it is likely that molecules that bind to Zmax1 may usefully alter bone development. Such molecules may include, for example, small molecules, proteins, RNA aptamers, peptide aptamers, and the like.

XVII. Preparation of Nucleic Acids, Vectors, Transformations and Host Cells

Large amounts of the nucleic acids of the present invention may be produced by replication in a suitable host cell. Natural or synthetic nucleic acid fragments coding for a desired fragment will be incorporated into recombinant nucleic acid constructs, usually DNA constructs, capable of introduction into and replication in a prokaryotic or eukaryotic cell. Usually the nucleic acid constructs will be suitable for replication in a unicellular host, such as yeast or bacteria, but may also be intended for introduction to (with and without integration within the genome) cultured mammalian or plant or other eukaryotic cell lines. The purification of nucleic acids produced by the methods of the present invention is described, for example, in Sambrook et al, Molecular Cloning. A Laboratory Manual, 2nd Ed. (Cold Spring Harbor Laboratory, Cold Spring Harbor, N.Y. (1989) or Ausubel et al, Current Protocols in Molecular Biology, J. Wiley and Sons, NY (1992).

The nucleic acids of the present invention may also be produced by chemical synthesis, e.g., by the phosphoramidite method described by Beaucage et al, Tetra. Letts., 22:1859-1862 (1981) or the triester method according to Matteucci, et al, J. Am. Chem. Soc., 103:3185 (1981), and may be performed on commercial, automated oligonucleotide synthesizers. A double-stranded fragment may be obtained from the single-stranded product of chemical synthesis either by synthesizing the complementary strand and annealing the strands together under appropriate conditions or by adding the complementary strand using DNA polymerase with an appropriate primer sequence.

Nucleic acid constructs prepared for introduction into a prokaryotic or eukaryotic host may comprise a replication system recognized by the host, including the intended nucleic acid fragment encoding the desired protein, and will preferably also include transcription and translational initiation regulatory sequences operably linked to the protein encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate, whether from a native HBM or Zmax1 protein or from other receptors or from secreted proteins of the same or related species, which allow the protein to cross and/or lodge in cell membranes, and thus attain its functional topology, or be secreted from the cell. Such vectors may be prepared by means of standard recombinant techniques well known in the art and discussed, for example, in Sambrook et al, Molecular Cloning. A Laboratory Manual, 2nd Ed. (Cold Spring Harbor Laboratory, Cold Spring Harbor, N.Y. (1989) or Ausubel et al, Current Protocols in Molecular Biology, J. Wiley and Sons, NY (1992).

An appropriate promoter and other necessary vector sequences will be selected so as to be functional in the host, and may include, when appropriate, those naturally associated with Zmax1 or HBM genes. Examples of workable combinations of cell lines and expression vectors are described in Sambrook et al, Molecular Cloning. A Laboratory Manual, 2nd Ed. (Cold Spring Harbor Laboratory, Cold Spring Harbor, N.Y. (1989) or Ausubel et al, Current Protocols in Molecular Biology, J. Wiley and Sons, NY (1992). Many useful vectors are known in the art and may be obtained from such vendors as Stratagene, New England BioLabs, Promega Biotech, and others. Promoters such as the trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. Vectors and promoters suitable for use in yeast expression are further described in EP 73,675A. Appropriate non-native mammalian promoters might include the early and late promoters from SV40 (Fiers et al, Nature, 273:113 (1978)) or promoters derived from murine Moloney leukemia virus, mouse tumor virus, avian sarcoma viruses, adenovirus II, bovine papilloma virus or polyoma. In addition, the construct may be joined to an amplifiable gene (e.g., DHFR) so that multiple copies of the gene may be made. For appropriate enhancer and other expression control sequences, see also Enhancers and Eukaryotic Gene Expression, Cold Spring Harbor Press, Cold Spring Harbor, N.Y. (1983).

While such expression vectors may replicate autonomously, they may also replicate by being inserted into the genome of the host cell, by methods well known in the art.

Expression and cloning vectors will likely contain a selectable marker, a gene encoding a protein necessary for survival or growth of a host cell transformed with the vector. The presence of this gene ensures growth of only those host cells which express the inserts. Typical selection genes encode proteins that a) confer resistance to antibiotics or other toxic substances, e.g. ampicillin, neomycin, methotrexate, etc.; b) complement auxotrophic deficiencies, or c) supply critical nutrients not available from complex media, e.g., the gene encoding D-alanine racemase for Bacilli. The choice of the proper selectable marker will depend on the host cell, and appropriate markers for different hosts are well known in the art.

The vectors containing the nucleic acids of interest can be transcribed in vitro, and the resulting RNA introduced into the host cell by well-known methods, e.g., by injection (see, Kubo et al, FEBS Letts. 241:119 (1988)), or the vectors can be introduced directly into host cells by methods well known in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. See generally, Sambrook et al., 1989 and Ausubel et al., 1992. The introduction of the nucleic acids into the host cell by any method known in the art, including those described above, will be referred to herein as “transformation.” The cells into which have been introduced nucleic acids described above are meant to also include the progeny of such cells.

Large quantities of the nucleic acids and proteins of the present invention may be prepared by expressing the Zmax1 or HBM nucleic acids or portions thereof in vectors or other expression vehicles in compatible prokaryotic or eukaryotic host cells. The most commonly used prokaryotic hosts are strains of Escherichia coli, although other prokaryotes, such as Bacillus subtilis or Pseudomonas may also be used.

Mammalian or other eukaryotic host cells, such as those of yeast, filamentous fungi, plant, insect, or amphibian or avian species, may also be useful for production of the proteins of the present invention. Propagation of mammalian cells in culture is per se well known. See, Jakoby and Pastan (eds.), Cell Culture. Methods in Enzymology, volume 58, Academic Press, Inc., Harcourt Brace Jovanovich, NY, (1979)). Examples of commonly used mammalian host cell lines are VERO and HeLa cells, Chinese hamster ovary (CHO) cells, and W138, BHK, and COS cell lines, although it will be appreciated by the skilled practitioner that other cell lines may be appropriate, e.g., to provide higher expression desirable glycosylation patterns, or other features.

Clones are selected by using markers depending on the mode of the vector construction. The marker may be on the same or a different DNA molecule, preferably the same DNA molecule. In prokaryotic hosts, the transformant may be selected, e.g., by resistance to ampicillin, tetracycline or other antibiotics. Production of a particular product based on temperature sensitivity may also serve as an appropriate marker.

Prokaryotic or eukaryotic cells transformed with the nucleic acids of the present invention will be useful not only for the production of the nucleic acids and proteins of the present invention, but also, for example, in studying the characteristics of Zmax1 or HBM proteins.

Antisense nucleic acid sequences are useful in preventing or diminishing the expression of Zmax1 or HBM, as will be appreciated by one skilled in the art. For example, nucleic acid vectors containing all or a portion of the Zmax1 or HBM gene or other sequences from the Zmax1 or HBM region may be placed under the control of a promoter in an antisense orientation and introduced into a cell. Expression of such an antisense construct within a cell will interfere with Zmax1 or HBM transcription and/or translation and/or replication.

The probes and primers based on the Zmax1 and HBM gene sequences disclosed herein are used to identify homologous Zmax1 and HBM gene sequences and proteins in other species. These Zmax1 and HBM gene sequences and proteins are used in the diagnostic/prognostic, therapeutic and drug screening methods described herein for the species from which they have been isolated.

XVIII. Protein Expression and Purification

Expression and purification of the HBM protein of the invention can be performed essentially as outlined below. To facilitate the cloning, expression and purification of membrane and secreted protein from the HBM gene, a gene expression system, such as the pET System (Novagen), for cloning and expression of recombinant proteins in E. coli was selected. Also, a DNA sequence encoding a peptide tag, the His-Tap, was fused to the 3′ end of DNA sequences of interest to facilitate purification of the recombinant protein products. The 3′ end was selected for fusion to avoid alteration of any 5′ terminal signal sequence.

Nucleic acids chosen, for example, from the nucleic acids set forth in SEQ ID NOS: 1, 3 and 5-11 for cloning HBM were prepared by polymerase chain reaction (PCR). Synthetic oligonucleotide primers specific for the 5′ and 3′ ends of the HBM nucleotide sequence were designed and purchased from Life Technologies (Gaithersburg, Md.). All forward primers (specific for the 5′ end of the sequence) were designed to include an NcoI cloning site at the 5′ terminus. These primers were designed to permit initiation of protein translation at the methionine residue encoded within the NcoI site followed by a valine residue and the protein encoded by the HBM DNA sequence. All reverse primers (specific for the 3′ end of the sequence) included an EcoRI site at the 5′ terminus to permit cloning of the HBM sequence into the reading frame of the pET-28b. The pET-28b vector provided a sequence encoding an additional 20 carboxyl-terminal amino acids including six histidine residues (at the C-terminus), which comprised the histidine affinity tag.

Genomic DNA prepared from the HBM gene was used as the source of template DNA for PCR amplification (Ausubel et al, Current Protocols in Molecular Biology, John Wiley & Sons (1994)). To amplify a DNA sequence containing the HBM nucleotide sequence, genomic DNA (50 ng) was introduced into a reaction vial containing 2 mM MgCl₂, 1 μM synthetic oligonucleotide primers (forward and reverse primers) complementary to and flanking a defined HBM, 0.2 mM of each of deoxynucleotide triphosphate, dATP, dGTP, dCTP, dTTP and 2.5 units of heat stable DNA polymerase (Amplitaq, Roche Molecular Systems, Inc., Branchburg, N.J.) in a final volume of 100 microliters.

Upon completion of thermal cycling reactions, each sample of amplified DNA was purified using the Qiaquick Spin PCR purification kit (Qiagen, Gaithersburg, Md.). All amplified DNA samples were subjected to digestion with the restriction endonucleases, e.g., NcoI and EcoRI (New England BioLabs, Beverly, Mass.) (Ausubel et al, Current Protocols in Molecular Biology, John Wiley & Sons, Inc. (1994)). DNA samples were then subjected to electrophoresis on 1.0% NuSeive (FMC BioProducts, Rockland, Me.) agarose gels. DNA was visualized by exposure to ethidium bromide and long wave UV irradiation. DNA contained in slices isolated from the agarose gel was purified using the Bio 101 GeneClean Kit protocol (Bio 101, Vista, Calif.).

The pET-28b vector was prepared for cloning by digestion with restriction endonucleases, e.g., NcoI and EcoRI (New England BioLabs, Beverly, Mass.) (Ausubel et al, Current Protocols in Molecular Biology, John Wiley & Sons, Inc. (1994)). The pET-28a vector, which encodes the histidine affinity tag that can be fused to the 5′ end of an inserted gene, was prepared by digestion with appropriate restriction endonucleases.

Following digestion, DNA inserts were cloned (Ausubel et al, Current Protocols in Molecular Biology, John Wiley & Sons, Inc. (1994)) into the previously digested pET-28b expression vector. Products of the ligation reaction were then used to transform the BL21 strain of E. coli (Ausubel et al, Current Protocols in Molecular Biology, John Wiley & Sons, Inc. (1994)) as described below.

Competent bacteria, E. coli strain BL21 or E. coli strain BL21 (DE3), were transformed with recombinant pET expression plasmids carrying the cloned HBM sequence according to standard methods (Ausubel et al, Current Protocols in Molecular Biology, John Wiley & Sons, Inc. (1994)). Briefly, 1 μl of ligation reaction was mixed with 50 μl of electrocompetent cells and subjected to a high voltage pulse, after which samples were incubated in 0.45 ml SOC medium (0.5% yeast extract, 2.0% tryptone, 10 mM NaCl, 2.5 mM KCl, 10 mM MgCl₂, 10 mM MgSO₄ and 20 mM glucose) at 37° C. with shaking for 1 hour. Samples were then spread on LB agar plates containing 25 μg/ml kanamycin sulfate for growth overnight. Transformed colonies of BL21 were then picked and analyzed to evaluate cloned inserts, as described below.

Individual BL21 clones transformed with recombinant pET-28b HBM nucleotide sequences were analyzed by PCR amplification of the cloned inserts using the same forward and reverse primers specific for the HBM sequences that were used in the original PCR amplification cloning reactions. Successful amplification verifies the integration of the HBM sequence in the expression vector (Ausubel et al, Current Protocols in Molecular Biology, John Wiley & Sons, Inc. (1994)).

Individual clones of recombinant pET-28b vectors carrying properly cloned HBM nucleotide sequences were picked and incubated in 5 ml of LB broth plus 25 μg/ml kanamycin sulfate overnight. The following day plasmid DNA was isolated and purified using the Qiagen plasmid purification protocol (Qiagen Inc., Chatsworth, Calif.).

The pET vector can be propagated in any E. coli K-12 strain, e.g., HMS174, HB101, JM109, DH5 and the like, for purposes of cloning or plasmid preparation. Hosts for expression include E. coli strains containing a chromosomal copy of the gene for T7 RNA polymerase. These hosts were lysogens of bacteriophage DE3, a lambda derivative that carries the lacI gene, the lacUV5 promoter and the gene for T7 RNA polymerase. T7 RNA polymerase was induced by addition of isopropyl-β-D-thiogalactoside (IPTG), and the T7 RNA polymerase transcribes any target plasmid containing a functional T7 promoter, such as pET-28b, carrying its gene of interest. Strains include, for example, BL21(DE3) (Studier et al, Meth. Enzymol., 185:60-89 (1990)).

To express the recombinant HBM sequence, 50 ng of plasmid DNA are isolated as described above to transform competent BL21(DE3) bacteria as described above (provided by Novagen as part of the pET expression kit). The lacZ gene (β-galactosidase) is expressed in the pET-System as described for the HBM recombinant constructions. Transformed cells were cultured in SOC medium for 1 hour, and the culture was then plated on LB plates containing 25 μg/ml kanamycin sulfate. The following day, the bacterial colonies were pooled and grown in LB medium containing kanamycin sulfate (25 μg/ml) to an optical density at 600 nM of 0.5 to 1.0 O.D. units, at which point 1 mM IPTG was added to the culture for 3 hours to induce gene expression of the HBM recombinant DNA constructions.

After induction of gene expression with IPTG, bacteria were collected by centrifugation in a Sorvall RC-3B centrifuge at 3500×g for 15 minutes at 4° C. Pellets were resuspended in 50 ml of cold mM Tris-HCl, pH 8.0, 0.1 M NaCl and 0.1 mM EDTA (STE buffer). Cells were then centrifuged at 2000×g for 20 minutes at 4° C. Wet pellets were weighed and frozen at −80° C. until ready for protein purification.

A variety of methodologies known in the art can be used to purify the isolated proteins (Coligan et al, Current Protocols in Protein Science, John Wiley & Sons (1995)). For example, the frozen cells can be thawed, resuspended in buffer and ruptured by several passages through a small volume microfluidizer (Model M-110S, Microfluidics International Corp., Newton, Mass.). The resultant homogenate is centrifuged to yield a clear supernatant (crude extract) and, following filtration, the crude extract is fractioned over columns. Fractions are monitored by absorbance at OD₂₈₀ nm and peak fractions may be analyzed by SDS-PAGE.

The concentrations of purified protein preparations are quantified spectrophotometrically using absorbance coefficients calculated from amino acid content (Perkins, Eur. J. Biochem., 157:169-180 (1986)). Protein concentrations are also measured by the method of Bradford, Anal. Biochem., 72:248-254 (1976) and Lowry et al, J. Biol. Chem., 193:265-275 (1951) using bovine serum albumin as a standard.

SDS-polyacrylamide gels of various concentrations were purchased from BioRad (Hercules, Calif.), and stained with Coomassie blue. Molecular weight markers may include rabbit skeletal muscle myosin (200 kDa), E. coli β-galactosidase (116 kDa), rabbit muscle phosphorylase B (97.4 kDa), bovine serum albumin (66.2 kDa), ovalbumin (45 kDa), bovine carbonic anyhdrase (31 kDa), soybean trypsin inhibitor (21.5 kDa), egg white lysozyme (14.4 kDa) and bovine aprotinin (6.5 kDa).

Once a sufficient quantity of the desired protein has been obtained, it may be used for various purposes. A typical use is the production of antibodies specific for binding. These antibodies may be either polyclonal or monoclonal, and may be produced by in vitro or in vivo techniques well known in the art. Monoclonal antibodies to epitopes of any of the peptides identified and isolated as described can be prepared from murine hybridomas (Kohler, Nature, 256:495 (1975)). In summary, a mouse is inoculated with a few micrograms of HBM protein over a period of two weeks. The mouse is then sacrificed. The cells that produce antibodies are then removed from the mouse's spleen. The spleen cells are then fused with polyethylene glycol with mouse myeloma cells. The successfully fused cells are diluted in a microtiter plate and growth of the culture is continued. The amount of antibody per well is measured by immunoassay methods such as ELISA (Engvall, Meth. Enzymol., 70:419 (1980)). Clones producing antibody can be expanded and further propagated to produce HBM antibodies. Other suitable techniques involve in vitro exposure of lymphocytes to the antigenic polypeptides, or alternatively, to selection of libraries of antibodies in phage or similar vectors. See Huse et al, Science, 246:1275-1281 (1989). For additional information on antibody production see Davis et al, Basic Methods in Molecular Biology, Elsevier, NY, Section 21-2 (1989).

XIX. Methods of Use: Gene Therapy

In recent years, significant technological advances have been made in the area of gene therapy for both genetic and acquired diseases. (Kay et al, Proc. Natl. Acad. Sci. USA, 94:12744-12746 (1997)) Gene therapy can be defined as the deliberate transfer of DNA for therapeutic purposes. Improvement in gene transfer methods has allowed for development of gene therapy protocols for the treatment of diverse types of diseases. Gene therapy has also taken advantage of recent advances in the identification of new therapeutic genes, improvement in both viral and nonviral gene delivery systems, better understanding of gene regulation, and improvement in cell isolation and transplantation.

The preceding experiments identify the HBM gene as a dominant mutation conferring elevated bone mass. The fact that this mutation is dominant indicates that expression of the HBM protein causes elevated bone mass. Older individuals carrying the HBM gene, and, therefore expressing the HBM protein, do not suffer from osteoporosis. These individuals are equivalent to individuals being treated with the HBM protein. These observations are a strong experimental indication that therapeutic treatment with the HBM protein prevents osteoporosis. The bone mass elevating activity of the HBM gene is termed “HBM function.”

Therefore, according to the present invention, a method is also provided of supplying HBM function to mesenchymal stem cells (Onyia et al, J. Bone Miner. Res., 13:20-30 (1998); Ko et al, Cancer Res., 56:4614-4619 (1996)). Supplying such a function provides protection against osteoporosis. The HBM gene or a part of the gene may be introduced into the cell in a vector such that the gene remains extrachromosomal. In such a situation, the gene will be expressed by the cell from the extrachromosomal location.

Vectors for introduction of genes both for recombination and for extrachromosomal maintenance are known in the art, and any suitable vector may be used. Methods for introducing DNA into cells such as electroporation, calcium phosphate co-precipitation, and viral transduction are known in the art, and the choice of method is within the competence of one skilled in the art (Robbins, Ed., Gene Therapy Protocols, Human Press, NJ (1997)). Cells transformed with the HBM gene can be used as model systems to study osteoporosis and drug treatments that promote bone growth.

As generally discussed above, the HBM gene or fragment, where applicable, may be used in gene therapy methods in order to increase the amount of the expression products of such genes in mesenchymal stem cells. It may be useful also to increase the level of expression of a given HBM protein, or a fragment thereof, even in those cells in which the wild type gene is expressed normally. Gene therapy would be carried out according to generally accepted methods as described by, for example, Friedman, Therapy for Genetic Diseases, Friedman, Ed., Oxford University Press, pages 105-121 (1991).

A virus or plasmid vector containing a copy of the HBM gene linked to expression control elements and capable of replicating inside mesenchymal stem cells, is prepared. Suitable vectors are known and described, for example, in U.S. Pat. No. 5,252,479 and WO 93/07282, the disclosures of which are incorporated by reference herein in their entirety. The vector is then injected into the patient, either locally into the bone marrow or systemically (in order to reach any mesenchymal stem cells located at other sites, i.e., in the blood). If the transfected gene is not permanently incorporated into the genome of each of the targeted cells, the treatment may have to be repeated periodically.

Gene transfer systems known in the art may be useful in the practice of the gene therapy methods of the present invention. These include viral and non-viral transfer methods. A number of viruses have been used as gene transfer vectors, including polyoma, i.e., SV40 (Madzak et al, J. Gen. Virol., 73:1533-1536 (1992)), adenovirus (Berkner, Curr. Top. Microbiol. Immunol., 158:39-61 (1992); Berkner et al, Bio Techniques, 6:616-629 (1988); Gorziglia et al, J. Virol., 66:4407-4412 (1992); Quantin et al, Proc. Natl. Acad. Sci. USA, 89:2581-2584 (1992); Rosenfeld et al, Cell, 68:143-155 (1992); Wilkinson et al, Nucl. Acids Res., 20:2233-2239 (1992); Stratford-Perricaudet et al, Hum. Gene Ther., 1:241-256 (1990)), vaccinia virus (Mackett et al, Biotechnology; 24:495-499 (1992)), adeno-associated virus (Muzyczka, Curr. Top. Microbiol. Immunol., 158:91-123 (1992); Ohi et al, Gene, 89:279-282,(1990)), herpes viruses including HSV and EBV (Margolskee, Curr. Top. Microbiol. Immunol., 158:67-90 (1992); Johnson et al, J. Virol., 66:2952-2965 (1992); Fink et al, Hum. Gene Ther., 3:11-19 (1992); Breakfield et al, Mol. Neurobiol., 1:337-371 (1987;) Fresse et al, Biochem. Pharmacol., 40:2189-2199 (1990)), and retroviruses of avian (Brandyopadhyay et al, Mol. Cell Biol., 4:749-754 (1984); Petropouplos et al, J. Virol., 66:3391-3397 (1992)), murine (Miller, Curr. Top. Microbiol. Immunol., 158:1-24 (1992); Miller et al, Mol. Cell Biol., 5:431-437 (1985); Sorge et al, Mol. Cell Biol., 4:1730-1737 (1984); Mann et al, J. Virol., 54:401-407 (1985)), and human origin (Page et al, J. Virol., 64:5370-5276 (1990); Buchschalcher et al, J. Virol., 66:2731-2739 (1992)). Most human gene therapy protocols have been based on disabled murine retroviruses.

Non-viral gene transfer methods known in the art include chemical techniques such as calcium phosphate coprecipitation (Graham et al, Virology, 52:456-467 (1973); Pellicer et al, Science, 209:1414-1422 (1980)), mechanical techniques, for example microinjection (Anderson et al, Proc. Natl. Acad. Sci. USA, 77:5399-5403 (1980); Gordon et al, Proc. Natl. Acad. Sci. USA, 77:7380-7384 (1980); Brinster et al, Cell, 27:223-231 (1981); Constantini et al, Nature, 294:92-94 (1981)), membrane fusion-mediated transfer via liposomes (Felgner et al, Proc. Natl. Acad. Sci. USA, 84:7413-7417 (1987); Wang et al, Biochemistry, 28:9508-9514 (1989); Kaneda et al, J. Biol. Chem., 264:12126-12129 (1989); Stewart et al, Hum. Gene Ther., 3:267-275 (1992); Nabel et al, Science, 249:1285-1288 (1990); Lim et al, Circulation, 83:2007-2011 (1992)), and direct DNA uptake- and receptor-mediated DNA transfer (Wolff et al, Science, 247:1465-1468 (1990); Wu et al, BioTechniques, 11:474-485 (1991); Zenke et al, Proc. Natl. Acad. Sci. USA, 87:3655-3659 (1990); Wu et al, J. Biol. Chem., 264:16985-16987 (1989); Wolff et al, BioTechniques, 11:474-485 (1991); Wagner et al, 1990; Wagner et al, Proc. Natl. Acad. Sci. USA, 88:4255-4259 (1991); Cotten et al, Proc. Natl. Acad. Sci. USA, 87:4033-4037 (1990); Curiel et al, Proc. Natl. Acad. Sci. USA, 88:8850-8854 (1991); Curiel et al, Hum. Gene Ther., 3:147-154 (1991)). Viral-mediated gene transfer can be combined with direct in vivo vectors to the mesenchymal stem cells and not into the surrounding cells (Romano et al, In Vivo, 12(1):59-67 (1998); Gonez et al, Hum. Mol. Genetics, 7(12):1913-9 (1998)). Alternatively, the retroviral vector producer cell line can be injected into the bone marrow (Culver et al, Science, 256:1550-1552 (1992)). Injection of producer cells would then provide a continuous source of vector particles. This technique has been approved for use in humans with inoperable brain tumors.

In an approach which combines biological and physical gene transfer methods, plasmid DNA of any size is combined with a polylysine-conjugated antibody specific to the adenovirus hexon protein, and the resulting complex is bound to an adenovirus vector. The trimolecular complex is then used to infect cells. The adenovirus vector permits efficient binding, internalization, and degradation of the endosome before the coupled DNA is damaged.

Liposome/DNA complexes have been shown to be capable of mediating direct in vivo gene transfer. While in standard liposome preparations the gene transfer process is non-specific, localized in vivo uptake and expression have been reported in tumor deposits, for example, following direct in situ administration (Nabel, Hum. Gene Ther., 3:399-410 (1992)).

XX. Methods of Use: Transformed Hosts, Development of Pharmaceuticals and Research Tools

Cells and animals that carry the HBM gene can be used as model systems to study and test for substances that have potential as therapeutic agents (Onyia et al, J. Bone Miner. Res., 13:20-30 (1998); Broder et al, Bone, 21:225-235 (1997)). The cells are typically cultured mesenchymal stem cells. These may be isolated from individuals with somatic or germline HBM genes. Alternatively, the cell line can be engineered to carry the HBM gene, as described above. After a test substance is applied to the cells, the transformed phenotype of the cell is determined. Any trait of transformed cells can be assessed, including formation of bone matrix in culture (Broder et al, Bone, 21:225-235 (1997)), mechanical properties (Kizer et al, Proc. Natl. Acad. Sci. USA, 94:1013-1018 (1997)), and response to application of putative therapeutic agents.

Animals for testing therapeutic agents can be selected after treatment of germline cells or zygotes. Such treatments include insertion of the Zmax1 gene, as well as insertion of the HBM gene and disrupted homologous genes. Alternatively, the inserted Zmax1 gene(s) and/or HBM gene(s) of the animals may be disrupted by insertion or deletion mutation of other genetic alterations using conventional techniques, such as those described by, for example, Capechi, Science, 244:1288 (1989); Valancuis et al, Mol. Cell Biol., 11:1402 (1991); Hasty et al, Nature, 350:243 (1991); Shinkai et al, Cell, 68:855 (1992); Mombaerts et al, Cell, 68:869 (1992); Philpott et al, Science, 256:1448 (1992); Snouwaert et al, Science, 257:1083 (1992); Donehower et al, Nature, 356:215 (1992). After test substances have been administered to the animals, the growth of bone must be assessed. If the test substance enhances the growth of bone, then the test substance is a candidate therapeutic agent. These animal models provide an extremely important vehicle for potential therapeutic products.

Individuals carrying the HBM gene have elevated bone mass. The HBM gene causes this phenotype by altering the activities, levels, expression patterns, and modification states of other molecules involved in bone development. Using a variety of established techniques, it is possible to identify molecules, preferably proteins or mRNAs, whose activities, levels, expression patterns, and modification states are different between systems containing the Zmax1 gene and systems containing the HBM gene. Such systems can be, for example, cell-free extracts, cells, tissues or living organisms, such as mice or humans. For a mutant form of Zmax1, a complete deletion of Zmax1, mutations lacking the extracellular or intracellular portion of the protein, or any other mutation in the Zmax1 gene may be used. It is also possible to use expression of antisense Zmax1 RNA or oligonucleotides to inhibit production of the Zmax1 protein. For a mutant form of HBM, a complete deletion of HBM, mutations lacking the extracellular or intracellular portion of the HBM protein, or any other mutation in the HBM gene may be used. It is also possible to use expression of antisense HBM RNA or oligonucleotides to inhibit production of the HBM protein.

Molecules identified by comparison of Zmax1 systems and HBM systems can be used as surrogate markers in pharmaceutical development or in diagnosis of human or animal bone disease. Alternatively, such molecules may be used in treatment of bone disease. See, Schena et al, Science, 270:467-470 (1995).

For example, a transgenic mouse carrying the HBM gene in the mouse homologue is constructed. A mouse of the genotype HBM/+is viable, healthy and has elevated bone mass. To identify surrogate markers for elevated bone mass, HBM/+(i.e., heterozygous) and isogenic +/+(i.e., wild-type) mice are sacrificed. Bone tissue mRNA is extracted from each animal, and a “gene chip” corresponding to mRNAs expressed in the +/+individual is constructed. mRNA from different tissues is isolated from animals of each genotype, reverse-transcribed, fluorescently labeled, and then hybridized to gene fragments affixed to a solid support. The ratio of fluorescent intensity between the two populations is indicative of the relative abundance of the specific mRNAs in the +/+ and HBM/+animals. Genes encoding mRNAs over- and under-expressed relative to the wild-type control are candidates for genes coordinately regulated by the HBM gene.

One standard procedure for identification of new proteins that are part of the same signaling cascade as an already-discovered protein is as follows. Cells are treated with radioactive phosphorous, and the already-discovered protein is manipulated to be more ore less active. The phosphorylation state of other proteins in the cell is then monitored by polyacrylamide gel electrophoresis and autoradiography, or similar techniques. Levels of activity of the known protein may be manipulated by many methods, including, for example, comparing wild-type mutant proteins using specific inhibitors such as drugs or antibodies, simply adding or not adding a known extracellular protein, or using antisense inhibition of the expression of the known protein (Tamura et al, Science, 280(5369):1614-7 (1998); Meng, EMBO J., 17(15):4391-403 (1998); Cooper et al, Cell, 1:263-73 (1982)).

In another example, proteins with different levels of phosphorylation are identified in TE85 osteosarcoma cells expressing either a sense or antisense cDNA for Zmax1. TE85 cells normally express high levels of Zmax1 (Dong et al, Biochem. & Biophys. Res. Comm., 251:784-790 (1998)). Cells containing the sense construct express even higher levels of Zmax1, while cells expressing the antisense construct express lower levels. Cells are grown in the presence of ³²P, harvested, lysed, and the lysates run on SDS polyacrylamide gels to separate proteins, and the gels subjected to autoradiography (Ausubel et al, Current Protocols in Molecular Biology, John Wiley & Sons (1997)). Bands that differ in intensity between the sense and antisense cell lines represent phosphoproteins whose phosphorylation state or absolute level varies in response to levels of Zmax1. As an alternative to the ³²P-labeling, unlabeled proteins may be separated by SDS-PAGE and subjected to immunoblotting, using the commercially available anti-phosphotyrosine antibody as a probe (Thomas et al, Nature, 376(6537):267-71 (1995)). As an alternative to the expression of antisense RNA, transfection with chemically modified antisense oligonucleotides can be used (Woolf et al, Nucleic Acids Res., 18(7):1763-9 (1990)).

Many bone disorders, such as osteoporosis, have a slow onset and a slow response to treatment. It is therefore useful to develop surrogate markers for bone development and mineralization. Such markers can be useful in developing treatments for bone disorders, and for diagnosing patients who may be at risk for later development of bone disorders. Examples of preferred markers are N- and C-terminal telopeptide markers described, for example, in U.S. Pat. Nos. 5,455,179, 5,641,837 and 5,652,112, the disclosures of which are incorporated by reference herein in their entirety. In the area of HIV disease, CD4 counts and viral load are useful surrogate markers for disease progression (Vlahov et al, JAMA, 279(1):35-40 (1998)). There is a need for analogous surrogate markers in the area of bone disease.

A surrogate marker can be any characteristic that is easily tested and relatively insensitive to non-specific influences. For example, a surrogate marker can be a molecule such as a protein or mRNA in a tissue or in blood serum. Alternatively, a surrogate marker may be a diagnostic sign such as sensitivity to pain, a reflex response or the like.

In yet another example, surrogate markers for elevated bone mass are identified using a pedigree of humans carrying the HBM gene. Blood samples are withdrawn from three individuals that carry the HBM gene, and from three closely related individuals that do not. Proteins in the serum from these individuals are electrophoresed on a two dimensional gel system, in which one dimension separates proteins by size, and another dimension separates proteins by isoelectric point (Epstein et al, Electrophoresis, 17(11):1655-70 (1996)). Spots corresponding to proteins are identified. A few spots are expected to be present in different amounts or in slightly different positions for the HBM individuals compared to their normal relatives. These spots correspond to proteins that are candidate surrogate markers. The identities of the proteins are determined by microsequencing, and antibodies to the proteins can be produced by standard methods for use in diagnostic testing procedures. Diagnostic assays for HBM proteins or other candidate surrogate markers include using antibodies described in this invention and a reporter molecule to detect HBM in human body fluids, membranes, bones, cells, tissues or extracts thereof. The antibodies can be labeled by joining them covalently or noncovalently with a substance that provides a detectable signal. In many scientific and patent literature, a variety of reporter molecules or labels are described including radionuclides, enzymes, fluorescent, chemi-luminescent or chromogenic agents (U.S. Pat. Nos. 3,817,837; 3,850,752; 3,939,350; 3,996,345; 4,277,437; 4,275,149; and 4,366,241).

Using these antibodies, the levels of candidate surrogate markers are measured in normal individuals and in patients suffering from a bone disorder, such as osteoporosis, osteoporosis pseudoglioma, Engelmann's disease, Ribbing's disease, hyperphosphatasemia, Van Buchem's disease, melorheostosis, osteopetrosis, pychodysostosis, sclerosteosis, osteopoikilosis, acromegaly, Paget's disease, fibrous dysplasia, tubular stenosis, osteogenesis imperfecta, hypoparathyroidism, pseudohypoparathyroidism, pseudopseudohypoparathyroidism, primary and secondary hyperparathyroidism and associated syndromes, hypercalciuria, medullary carcinoma of the thyroid gland, osteomalacia and other diseases. Techniques for measuring levels of protein in serum in a clinical setting using antibodies are well established. A protein that is consistently present in higher or lower levels in individuals carrying a particular disease or type of disease is a useful surrogate marker.

A surrogate marker can be used in diagnosis of a bone disorder. For example, consider a child that present to a physician with a high frequency of bone fracture. The underlying cause may be child abuse, inappropriate behavior by the child, or a bone disorder. To rapidly test for a bone disorder, the levels of the surrogate marker protein are measured using the antibody described above.

Levels of modification states of surrogate markers can be measured as indicators of the likely effectiveness of a drug that is being developed. It is especially convenient to use surrogate markers in creating treatments for bone disorders, because alterations in bone development or mineralization may require a long time to be observed. For example, a set of bone mRNAs, termed the “HBM-inducible mRNA set” is found to be overexpressed in HBM/+mice as compared to +/+mice, as described above. Expression of this set can be used as a surrogate marker. Specifically, if treatment of +/+mice with a compound results in overexpression of the HBM-inducible mRNA set, then that compound is considered a promising candidate for further development.

This invention is particularly useful for screening compounds by using the Zmax1 or HBM protein or binding fragment thereof in any of a variety of drug screening techniques.

The Zmax1 or HBM protein or fragment employed in such a test may either be free in solution, affixed to a solid support, or borne on a cell surface. One method of drug screening utilizes eukaryotic or prokaryotic host cells which are stably transformed with recombinant nucleic acids expressing the protein or fragment, preferably in competitive binding assays. Such cells, either in viable or fixed form, can be used for standard binding assays. One may measure, for example, for the formation of complexes between a Zmax1 or HBM protein or fragment and the agent being tested, or examine the degree to which the formation of a complex between a Zmax1 or HBM protein or fragment and a known ligand is interfered with by the agent being tested.

Thus, the present invention provides methods of screening for drugs comprising contacting such an agent with a Zmax1 or HBM protein or fragment thereof and assaying (i) for the presence of a complex between the agent and the Zmax1 or HBM protein or fragment, or (ii) for the presence of a complex between the Zmax1 or HBM protein or fragment and a ligand, by methods well known in the art. In such competitive binding assays the Zmax1 or HBM protein or fragment is typically labeled. Free Zmax1 or HBM protein or fragment is separated from that present in a protein:protein complex, and the amount of free (i.e., uncomplexed) label is a measure of the binding of the agent being tested to Zmax1 or HBM or its interference with Zmax1 or HBM: ligand binding, respectively.

Another technique for drug screening provides high throughput screening for compounds having suitable binding affinity to the Zmax1 or HBM proteins and is described in detail in WO 84/03564. Briefly stated, large numbers of different small peptide test compounds are synthesized on a solid substrate, such as plastic pins or some other surface. The peptide test compounds are reacted with. Zmax1 or HBM proteins and washed. Bound Zmax1 or HBM protein is then detected by methods well known in the art. Purified Zmax1 or HBM can be coated directly onto plates for use in the aforementioned drug screening techniques. However, non-neutralizing antibodies to the protein can be used to capture antibodies to immobilize the Zmax1 or HBM protein on the solid phase.

This invention also contemplates the use of competitive drug screening assays in which neutralizing antibodies capable of specifically binding the Zmax1 or HBM protein compete with a test compound for binding to the Zmax1 or HBM protein or fragments thereof. In this manner, the antibodies can be used to detect the presence of any peptide that shares one or more antigenic determinants of the Zmax1 or HBM protein.

A further technique for drug screening involves the use of host eukaryotic cell lines or cells (such as described above) that have a nonfunctional Zmax1 or HBM gene. These host cell lines or cells are defective at the Zmax1 or HBM protein level. The host cell lines or cells are grown in the presence of drug compound. The rate of growth of the host cells is measured to determine if the compound is capable of regulating the growth of Zmax1 or HBM defective cells.

The goal of rational drug design is to produce structural analogs of biologically active proteins of interest or of small molecules with which they interact (e.g., agonists, antagonists, inhibitors) in order to fashion drugs which are, for example, more active or stable forms of the protein, or which, e.g., enhance or interfere with the function of a protein in vivo. See, e.g., Hodgson, Bio/Technology, 9:19-21 (1991). In one approach, one first determines the three-dimensional structure of a protein of interest (e.g., Zmax1 or HBM protein) or, for example, of the Zmax1- or HBM-receptor or ligand complex, by x-ray crystallography, by computer modeling or most typically, by a combination of approaches. Less often, useful information regarding the structure of a protein may be gained by modeling based on the structure of homologous proteins. An example of rational drug design is the development of HIV protease inhibitors (Erickson et al, Science, 249:527-533 (1990)). In addition, peptides (e.g., Zmax1 or HBM protein) are analyzed by an alanine scan (Wells, Methods in Enzymol., 202: 390-411 (1991)). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacore.

Thus, one may design drugs which have, e.g., improved Zmax1 or HBM protein activity or stability or which act as inhibitors, agonists, antagonists, etc. of Zmax1 or HBM protein activity. By virtue of the availability of cloned Zmax1 or HBM sequences, sufficient amounts of the Zmax1 or HBM protein may be made available to perform such analytical studies as x-ray crystallography. In addition, the knowledge of the Zmax1 or HBM protein sequence provided herein will guide those employing computer modeling techniques in place of, or in addition to x-ray crystallography.

XXI. Methods of Use: Avian and Mammalian Animal Husbandry

The Zmax1 DNA and Zmax1 protein and/or the HBM DNA and HBM protein can be used for vertebrate and preferably human therapeutic agents and for avian and mammalian veterinary agents, including for livestock breeding. Birds, including, for example, chickens, roosters, hens, turkeys, ostriches, ducks, pheasants and quails, can benefit from the identification of the gene and pathway for high bone mass. In many examples cited in literature (for example, McCoy et al, Res. Vet. Sci., 60(2): 185-186 (1996)), weakened bones due to husbandry conditions cause cage layer fatigue, osteoporosis and high mortality rates. Additional therapeutic agents to treat osteoporosis or other bone disorders in birds can have considerable beneficial effects on avian welfare and the economic conditions of the livestock industry, including, for example, meat and egg production.

XXII. Methods of Use: Diagnostic Assays using Zmax1-Specific Oligonucleotides for Detection of Genetic Alterations Affecting Bone Development.

In cases where an alteration or disease of bone development is suspected to involve an alteration of the Zmax1 gene or the HBM gene, specific oligonucleotides may be constructed and used to assess the level of Zmax1 mRNA or HBM mRNA, respectively, in bone tissue or in another tissue that affects bone development.

For example, to test whether a person has the HBM gene, which affects bone density, polymerase chain reaction can be used. Two oligonucleotides are synthesized by standard methods or are obtained from a commercial supplier of custom-made oligonucleotides. The length and base composition are determined by standard criteria using the Oligo 4.0 primer Picking program (Wojchich Rychlik, 1992). One of the oligonucleotides is designed so that it will hybridize only to HBM DNA under the PCR conditions used. The other oligonucleotide is designed to hybridize a segment of Zmax1 genomic DNA such that amplification of DNA using these oligonucleotide primers produces a conveniently identified DNA fragment. For example, the pair of primers CCAAGTTCTGAGAAGTCC (SEQ ID NO:32) and AATACCTGAAACCATACCTG (SEQ ID NO:33) will amplify a 530 base pair DNA fragment from a DNA sample when the following conditions are used: step 1 at 95° C. for 120 seconds; step 2 at 95° C. for 30 seconds; step 3 at 58° C. for 30 seconds; step 4 at 72° C. for 120 seconds; where steps 2-4 are repeated 35 times. Tissue samples may be obtained from hair follicles, whole blood, or the buccal cavity.

The fragment generated by the above procedure is sequenced by standard techniques. Individuals heterozygous for the HBM gene will show an equal amount of G and T at the second position in the codon for glycine 171. Normal or homozygous wild-type individuals will show only G at this position. Other amplification techniques besides PCR may be used as alternatives, such as ligation-mediated PCR or techniques involving Q-beta replicase (Cahill et al, Clin. Chem., 37(9):1482-5 (1991)). For example, the oligonucleotides AGCTGCTCGT AGCTG TCTCTCCCTGGATCACGGGTACATGTACTGGACAGACTGGGT (SEQ ID NO:34) and TGAGACGCCCCGGATTGAGCGGGCAGGGATAGCTTA TTCCCTGTGCCGCATTACGGC (SEQ ID NO:35) can be hybridized to a denatured human DNA sample, treated with a DNA ligase, and then subjected to PCR amplification using the primer oligonucleotides AGCTGCTCGTAGCTGTCT CTCCCTGGA (SEQ ID NO:36) and GCCGTAATGCGGCACAGGGAATAAGCT (SEQ ID NO:37). In the first two oligonucleotides, the outer 27 bases are random sequence corresponding to primer binding sites, and the inner 30 bases correspond to sequences in the Zmax1 gene. The T at the end of the first oligonucleotide corresponds to the HBM gene. The first two oligonucleotides are ligated only when hybridized to human DNA carrying the HBM gene, which results in the formation of an amplifiable 114 bp DNA fragment.

Products of amplification can be detected by agarose gel electrophoresis, quantitative hybridization, or equivalent techniques for nucleic acid detection known to one skilled in the art of molecular biology (Sambrook et al, Molecular Cloning: A Laboratory Manual, Cold Spring Harbor Laboratory, Cold Spring, N.Y. (1989)).

Other alterations in the Zmax1 gene or the HBM gene may be diagnosed by the same type of amplification-detection procedures, by using oligonucleotides designed to identify those alterations. These procedures can be used in animals as well as humans to identify alterations in Zmax1 or HBM that affect bone development.

Expression of Zmax1 or HBM in bone tissue may be accomplished by fusing the cDNA of Zmax1 or HBM, respectively, to a bone-specific promoter in the context of a vector for genetically engineering vertebrate cells. DNA constructs are introduced into cells by packaging the DNA into virus capsids, by the use of cationic liposomes, electroporation, or by calcium phosphate transfection. Transfected cells, preferably osteoblasts, may be studied in culture or may be introduced into bone tissue in animals by direct injection into bone or by intravenous injection of osteoblasts, followed by incorporation into bone tissue (Ko et al, Cancer Research, 56(20):4614-9 (1996)). For example, the osteocalcin promoter, which is specifically active in osteoblasts, may be used to direct transcription of the Zmax1 gene or the HBM gene. Any of several vectors and transfection methods may be used, such as retroviral vectors, adenovirus vectors, or vectors that are maintained after transfection using cationic liposomes, or other methods and vectors described herein.

Alteration of the level of functional Zmax1 protein or HBM protein affects the level of bone mineralization. By manipulating levels of functional Zmax1 protein or HBM protein, it is possible to affect bone development and to increase or decrease levels of bone mineralization. For example, it may be useful to increase bone mineralization in patients with osteoporosis. Alternatively, it may be useful to decrease bone mineralization in patients with osteopetrosis or Paget's disease. Alteration of Zmax1 levels or HBM levels can also be used as a research tool. Specifically, it is possible to identify proteins, mRNA and other molecules whose level or modification status is altered in response to changes in functional levels of Zmax1 or HBM. The pathology and pathogenesis of bone disorders is known and described, for example, in Rubin and Farber (Eds.), Pathology, 2nd Ed., S. B. Lippincott Co., Philadelphia, Pa. (1994).

A variety of techniques can be used to alter the levels of functional Zmax1 or HBM. For example, intravenous or intraosseous injection of the extracellular portion of Zmax1 or mutations thereof, or HBM or mutations thereof, will alter the level of Zmax1 activity or HBM activity, respectively, in the body of the treated human, animal or bird. Truncated versions of the Zmax1 protein or HBM protein can also be injected to alter the levels of functional Zmax1 protein or HBM protein, respectively. Certain forms of Zmax1 or HBM enhance the activity of endogenous protein, while other forms are inhibitory.

In a preferred embodiment, the HBM protein is used to treat osteoporosis. In a further preferred embodiment, the extracellular portion of the HBM protein is used. This HBM protein may be optionally modified by the addition of a moiety that causes the protein to adhere to the surface of cells. The protein is prepared in a pharmaceutically acceptable solution and is administered by injection or another method that achieves acceptable pharmacokinetics and distribution.

In a second embodiment of this method, Zmax1 or HBM levels are increased or decreased by gene therapy techniques. To increase Zmax1 or HBM levels, osteoblasts or another useful cell type are genetically engineered to express high levels of Zmax1 or HBM as described above. Alternatively, to decrease Zmax1 or HBM levels, antisense constructs that specifically reduce the level of translatable Zmax1 or HBM mRNA can be used. In general, a tissue-nonspecific promoter may be used, such as the CMV promoter or another commercially available promoter found in expression vectors (Wu et al, Toxicol. Appl. Pharmacol., 141(1):330-9 (1996)). In a preferred embodiment, a Zmax1 cDNA or its antisense is transcribed by a bone-specific promoter, such as the osteocalcin or another promoter, to achieve specific expression in bone tissue. In this way, if a Zmax1-expressing DNA construct or HBM-expressing construct is introduced into non-bone tissue, it will not be expressed.

In a third embodiment of this method, antibodies against Zmax1 or HBM are used to inhibit its function. Such antibodies are identified herein.

In a fourth embodiment of this method, drugs that inhibit Zmax1 function or HBM function are used. Such drugs are described herein and optimized according to techniques of medicinal chemistry well known to one skilled in the art of pharmaceutical development.

Zmax1 and HBM interact with several proteins, such as ApoE. Molecules that inhibit the interaction between Zmax1 or HBM and ApoE or another binding partner are expected to alter bone development and mineralization. Such inhibitors may be useful as drugs in the treatment of osteoporosis, osteopetrosis, or other diseases of bone mineralization. Such inhibitors may be low molecular weight compounds, proteins or other types of molecules. See, Kim et al, J. Biochem. (Tokyo), 124(6):1072-1076 (1998).

Inhibitors of the interaction between Zmax1 or HBM and interacting proteins may be isolated by standard drug-screening techniques. For example, Zmax1 protein, (or a fragment thereof) or HBM protein (or a fragment thereof) can be immobilized on a solid support such as the base of microtiter well. A second protein or protein fragment, such as ApoE is derivatized to aid in detection, for example with fluorescein. Iodine, or biotin, then added to the Zmax1 or HBM in the presence of candidate compounds that may specifically inhibit this protein-protein domain of Zmax1 or HBM, respectively, and thus avoid problems associated with its transmembrane segment. Drug screens of this type are well known to one skilled in the art of pharmaceutical development.

Because Zmax1 and HBM are involved in bone development, proteins that bind to Zmax1 and HBM are also expected to be involved in bone development. Such binding proteins can be identified by standard methods, such as co-immunoprecipitation, co-fractionation, or the two-hybrid screen (Ausubel et al, Current Protocols in Molecular Biology, John Wiley & Sons (1997)). For example, to identify Zmax1-interacting proteins or HBM-interacting proteins using the two-hybrid system, the extracellular domain of Zmax1 or HBM is fused to LexA and expressed for the yeast vector pEG202 (the “bait”) and expressed in the yeast strain EGY48. The yeast strain is transformed with a “prey” library in the appropriate vector, which encodes a galactose-inducible transcription-activation sequence fused to candidate interacting proteins. The techniques for initially selecting and subsequently verifying interacting proteins by this method are well known to one skilled in the art of molecular biology (Ausubel et al, Current Protocols in Molecular Biology, John Wiley & Sons (1997)).

In a preferred embodiment, proteins that interact with HBM, but not Zmax1, are identified using a variation of the above procedure (Xu et al, Proc. Natl. Acad. Sci USA, 94(23):12473-8 (November 1997)). This variation of the two-hybrid system uses two baits, and Zmax1 and HBM are each fused to LexA and TetR, respectively. Alternatively, proteins that interact with the HBM but not Zmax1 are also isolated. These procedures are well known to one skilled in the art of molecular biology, and are a simple variation of standard two-hybrid procedures.

As an alternative method of isolating Zmax1 or HBM interacting proteins, a biochemical approach is used. The Zmax1 protein or a fragment thereof, such as the extracellular domain, or the HBM protein or a fragment thereof, such as the extracellular domain, is chemically coupled to Sepharose beads. The Zmax1- or HBM-coupled beads are poured into a column. An extract of proteins, such as serum proteins, proteins in the supernatant of a bone biopsy, or intracellular proteins from gently lysed TE85 osteoblastic cells, is added to the column. Non-specifically bound proteins are eluted, the column is washed several times with a low-salt buffer, and then tightly binding proteins are eluted with a high-salt buffer. These are candidate proteins that bind to Zmax1 or HBM, and can be tested for specific binding by standard tests and control experiments. Sepharose beads used for coupling proteins and the methods for performing the coupling are commercially available (Sigma), and the procedures described here are well known to one skilled in the art of protein biochemistry.

As a variation of the above procedure, proteins that are eluted by high salt from the Zmax1- or HBM-Sepharose column are then added to an HBM-Zmax1-sepharose column. Proteins that flow through without sticking are proteins that bind to Zmax1 but not to HBM. Alternatively, proteins that bind to the HBM protein and not to the Zmax1 protein can be isolated by reversing the order in which the columns are used.

XXIII. Method of Use: Transformation-Associated Recombination (TAR) Cloning

Essential for the identification of novel allelic variants of Zmax1 is the ability to examine the sequence of both copies of the gene in an individual. To accomplish this, two “hooks,” or regions of significant similarity, are identified within the genomic sequence such that they flank the portion of DNA that is to be cloned. Most preferably, the first of these hooks is derived from sequences 5′ to the first exon of interest and the second is derived from sequences 3′ to the last exon of interest. These two “hooks” are cloned into a bacterial/yeast shuttle vector such as that described by Larionov et al, Proc. Natl. Acad. Sci. USA, 94:7384-7387 (1997). Other similar vector systems may also be used. To recover the entire genomic copy of the Zmax1 gene, the plasmid containing the two “hooks” is linearized with a restriction endonuclease or is produced by another method such as PCR. This linear DNA fragment is introduced into yeast cells along with human genomic DNA. Typically, the yeast Saccharomyces cerevisiae is used as a host cell, although Larionov et al (in press) have reported using chicken host cells as well. During and after the process of transformation, the endogenous host cell converts the linear plasmid to a circle by a recombination event whereby the region of the human genomic DNA homologous to the “hooks” is inserted into the plasmid. This plasmid can be recovered and analyzed by methods well known to one skilled in the art. Obviously, the specificity for this reaction requires the host cell machinery to recognize sequences similar to the “hooks” present in the linear fragment. However, 100% sequence identity is not required, as shown by Kouprina et al, Genomics, 53(1):21-28 (October 1998), where the author describes using degenerate repeated sequences common in the human genome to recover fragments of human DNA from a rodent/human hybrid cell line.

In another example, only one “hook” is required, as described by Larionov et al, Proc. Natl. Acad. Sci. USA, 95(8):4469-74 (April 1998). For this type of experiment, termed “radial TAR cloning,” the other region of sequence similarity to drive the recombination is derived from a repeated sequence from the genome. In this way, regions of DNA adjacent to the Zmax1 gene coding region can be recovered and examined for alterations that may affect function.

XXIV. Methods of Use: Genomic Screening

The use of polymorphic genetic markers linked to the HBM gene or to Zmax1 is very useful in predicting susceptibility to osteoporosis or other bone diseases. Koller et al, Amer. J Bone Min. Res., 13:1903-1908 (1998) have demonstrated that the use of polymorphic genetic markers is useful for linkage analysis. Similarly, the identification of polymorphic genetic markers within the high bone mass gene will allow the identification of specific allelic variants that are in linkage disequilibrium with other genetic lesions that affect bone development. Using the DNA sequence from the BACs, a dinucleotide CAn repeat was identified and two unique PCR primers that will amplify the genomic DNA containing this repeat were designed, as shown below:

| | | |
|---|---|---|
| B200E21C16_L: | GAGAGGCTATATCCCTGGGC | (SEQ ID NO:38) |
| B200E21C16_R: | ACAGCACGTGTTTAAAGGGG | (SEQ ID NO:39) |

and used in the genetic mapping study.

This method has been used successfully by others skilled in the art (e.g., Sheffield et al, Genet., 4:1837-1844 (1995); LeBlanc-Straceski et al, Genomics, 19:341-9 (1994); Chen et al, Genomics, 25:1-8 (1995)). Use of these reagents, with populations or individuals will predict their risk for osteoporosis. Similarly, single nucleotide polymorphisms (SNPs), such as those shown in Table 4 above, can be used as well to predict risk for developing bone diseases or resistance to osteoporosis in the case of the HBM gene.

XXV. Methods of Use: Modulators of Tissue Calcification

The calcification of tissues in the human body is well documented. Towler et al, J. Biol. Chem., 273:30427-34 (1998) demonstrated that several proteins known to regulate calcification of the developing skull in a model system are expressed in calcified aorta. The expression of Msx2, a gene transcribed in osteoprogenitor cells, in calcified vascular tissue indicates that genes which are important in bone development are involved in calcification of other tissues. Treatment with HBM protein, agonists or antagonists is likely to ameliorate calcification (such as the vasculature, dentin and bone of the skull visera) due to its demonstrated effect on bone mineral density. In experimental systems where tissue calcification is demonstrated, the over-expression or repression of Zmax1 activity permits the identification of molecules that are directly regulated by the Zmax1 gene. These genes are potential targets for therapeutics aimed at modulating tissue calcification. For example, an animal, such as the LDLR −/−, mouse is fed a high fat diet and is observed to demonstrate expression of markers of tissue calcification, including Zmax1. These animals are then treated with antibodies to Zmax1 or HBM protein, antisense oligonucleotides directed against Zmax1 or HBM cDNA, or with compounds known to bind the Zmax1 or HBM protein or its binding partner or ligand. RNA or proteins are extracted from the vascular tissue and the relative expression levels of the genes expressed in the tissue are determined by methods well known in the art. Genes that are regulated in the tissue are potential therapeutic targets for pharmaceutical development as modulators of tissue calcification.

The nucleic acids, proteins, peptides, amino acids, small molecules or other pharmaceutically useful compounds of the present invention that are to be given to an individual may be administered in the form of a composition with a pharmaceutically acceptable carrier, excipient or diluent, which are well known in the art. The individual may be a mammal or a bird, preferably a human, a rat, a mouse or bird. Such compositions may be administered to an individual in a pharmaceutically effective amount. The amount administered will vary depending on the condition being treated and the patient being treated. The compositions may be administered alone or in combination with other treatments.

### EXAMPLES

The present invention is described by reference to the following Examples, which are offered by way of illustration and are not intended to limit the invention in any manner. Standard techniques well known in the art or the techniques specifically described below were utilized.

### Example 1

The propositus was referred by her physicians to the Creighton Osteoporosis Center for evaluation of what appeared to be unusually dense bones. She was 18 years old and came to medical attention two years previous because of back pain, which was precipitated by an auto accident in which the car in which she was riding as a passenger was struck from behind. Her only injury was soft tissue injury to her lower back that was manifested by pain and muscle tenderness. There was no evidence of fracture or subluxation on radiographs. The pain lasted for two years, although she was able to attend school full time. By the time she was seen in the Center, the pain was nearly resolved and she was back to her usual activities as a high school student. Physical exam revealed a normal healthy young woman standing 66 inches and weighing 128 pounds. Radiographs of the entire skeleton revealed dense looking bones with thick cortices. All bones of the skeleton were involved. Most importantly, the shapes of all the bones were entirely normal. The spinal BMC was 94.48 grams in L1-4, and the spinal BMD was 1.667 gm/cm² in L1-4. BMD was 5.62 standard deviations (SD) above peak skeletal mass for women. These were measured by DXA using a Hologic 2000˜. Her mother was then scanned and a lumbar spinal BMC of 58.05 grams and BMD of 1.500 gm/cm² were found. Her mother's values place her 4.12 SD above peak mass and 4.98 SD above her peers. Her mother was 51 years old, stood 65 inches and weighed 140 pounds. Her mother was in excellent health with no history of musculoskeletal or other symptoms. Her father's lumbar BMC was 75.33 grams and his BMD was 1.118 gm/cm². These values place him 0.25 SD above peak bone mass for males. He was in good health, stood 72 inches tall, and weighed 187 pounds.

These clinical data suggested that the propositus inherited a trait from her mother, which resulted in very high bone mass, but an otherwise normal skeleton, and attention was focused on the maternal kindred. In U.S. Pat. No. 5,691,153, twenty-two of these members had measurement of bone mass by DXA. In one case, the maternal grandfather of the propositus, was deceased, however, medical records, antemortem skeletal radiographs and a gall bladder specimen embedded in paraffin for DNA genotyping were obtained. His radiographs showed obvious extreme density of all of the bones available for examination including the femur and the spine, and he was included among the affected members. In this invention, the pedigree has been expanded to include 37 informative individuals. These additions are a significant improvement over the original kinship (Johnson et al, Am. J. Hum. Genet., 60:1326-1332 (1997)) because, among the fourteen individuals added since the original study, two individuals harbor key crossovers. X-linkage is ruled out by the presence of male-to-male transmission from individual 12 to 14 and 15.

### Example 2

The present invention describes DNA sequences derived from two BAC clones from the HBM gene region, as evident in Table 7 below, which is an assembly of these clones. Clone b200e21-h (ATCC No. 980812; SEQ ID NOS: 10-11) was deposited at the American Type Culture Collection (ATCC), 10801 University Blvd., Manassas, Va. 20110-2209 U.S.A., on Dec. 30, 1997. Clone b527d12-h (ATCC No. 980720; SEQ ID NOS: 5-9) was deposited at the American Type Culture Collection (ATCC), 10801 University Blvd., Manassas, Va. 20110-2209 U.S.A., on Oct. 2, 1998. These sequences are unique reagents that can be used by one skilled in the art to identify DNA probes for the Zmax1 gene, PCR primers to amplify the gene, nucleotide polymorphisms in the Zmax1 gene, or regulatory elements of the Zmax1 gene.

TABLE 7

| | | SEQ ID | Length |
|---|---|---|---|
| Contig | ATCC No. | NO. | (base pairs) |
| b527d12-h_contig302G | 980720 | 5 | 3096 |
| b527d12-h_contig306G | 980720 | 6 | 26928 |
| b527d12-h_contig307G | 980720 | 7 | 29430 |
| b527d12-h_contig308G | 980720 | 8 | 33769 |
| b527d12-h_contig309G | 980720 | 9 | 72049 |
| b200e21-h_contig1 | 980812 | 10 | 8705 |
| b200e21-h_contig4 | 980812 | 11 | 66933 |

The disclosure of each of the patents, patent applications and publications cited in the specification is hereby incorporated by reference herein in its entirety.

Although the invention has been set forth in detail, one skilled in the art will recognize that numerous changes and modifications can be made, and that such changes and modifications may be made without departing from the spirit and scope of the invention.

This application claims priority to U.S. application Ser. Nos. 09/543,771 and 09/544,398 filed on Apr. 5, 2000, which are a continuation-in-part of application Ser. No. 09/229,319, filed Jan. 13, 1999, which claims benefit of U.S. Provisional Application No. 60/071,449, filed Jan. 13, 1998, and U.S. Provisional Application No. 60/105,511, filed Oct. 23, 1998, all of which are herein incorporated by reference in their entirety.

What is claimed is:

1. An isolated nucleic acid sequence of SEQ ID NO: 2.

2. The isolated nucleic acid sequence of claim 1, wherein the nucleic acid sequence is DNA.

3. An isolated amino acid sequence of SEQ ID NO: 4.

4. A nucleic acid sequence encoding the amino acid sequence of SEQ ID NO:4.

5. A replicative cloning vector comprising the nucleic acid sequence of claim 1 and a replicon operative in an isolated host cell.

6. An isolated host cell transformed with the replicative cloning vector of claim 5.

7. An expression vector comprising the nucleic acid sequence of claim 1 operably linked to a transcription regulatory region.

8. An isolated host cell transformed with the expression vector of claim 7.

9. A method for testing a substance as a therapeutic agent for bone modulation in a host comprising administering the nucleic acid of claim 1 to the host, and assessing whether bone modulation occurs.

10. The method of claim 9, wherein the host is a cell or an animal.

11. The method of claim 10, wherein the animal is a human, a rodent or a bird.

12. A method of identifying a molecule involved in bone modulation comprising identifying a molecule that binds to, or that inhibits binding of a molecule to, HBM.

13. The method of claim 12, wherein said molecule is a protein.

14. A method for identifying a protein involved in bone modulation comprising identifying a protein that has an expression level that is different in a first host comprising the Zmax1 gene when compared to a second host comprising the HBM gene.

15. The method of claim 14, wherein the host is a cell or an animal.

16. A method of identifying a candidate protein involved in bone modulation comprising
identifying a protein in a first individual having the high bone mass phenotype;
identifying a protein in a second individual not having the high bone mass phenotype;
comparing the protein of the first individual to the protein of the second individual, wherein (i) the protein that is present in the first individual but not the second individual is the candidate protein or (ii) the protein that is present in a higher amount in the first individual than in the second individual is the candidate protein or (iii) the protein that is present in a lower amount in the first individual than in the second individual is the candidate protein.

17. The method of claim 16, further comprising producing an antibody to the candidate protein.

18. A method of identifying a candidate protein involved in bone modulation comprising
identifying a protein in a first individual having the high bone mass phenotype;
identifying a protein in a second individual not having the high bone mass phenotype; and
comparing the protein of the first individual to the protein of the second individual, wherein (i) the protein that is present in the second individual but not the first individual is the candidate protein or (ii) the protein that is present in a higher amount in the second individual than in the first individual is the candidate protein or (iii) the protein that is present in a lower amount in the second individual than in the first individual is the candidate protein.

19. The method of claim 18, further comprising producing an antibody to the candidate protein.

20. A method of testing for HBM activity comprising immobilizing an HBM protein, binding a protein to the HBM protein, and measuring the extent of binding.

21. The method of claim 20, wherein the protein is ApoE.

22. A method for identification of a candidate molecule involved in bone modulation comprising
identifying a molecule that binds to, or that inhibits binding of a molecule to, the nucleic acid sequence of SEQ ID NO: 1;
identifying a molecule that binds to, or that inhibits binding of a molecule to, the nucleic acid sequence of SEQ ID NO: 2; and
comparing the extent of binding, or the extent of inhibition of binding, of the molecule to each nucleic acid sequence, wherein the molecule that binds, or inhibits binding, more or less to the nucleic acid sequence of SEQ ID NO: 2 or the nucleic acid sequence of SEQ ID NO: 1 is the candidate molecule.

23. The method of claim 22, wherein the candidate molecule is a protein or an mRNA.

24. A method of pharmaceutical development for treatment of bone development disorders comprising identifying a molecule that binds to the amino acid sequence of SEQ ID NO: 4.

25. The method of claim 24, wherein the molecule inhibits or enhances the function of the amino acid.

26. A method of pharmaceutical development for treatment of bone development disorders comprising
constructing a first host that contains the Zmax1 gene or protein;
constructing a second host that contains the HBM gene or protein;
analyzing a difference between the first host and the second host;
identifying a molecule that, when added to the first host, causes the first host to exhibit a characteristic feature of the second host.

27. The method of claim 26, wherein the host is a cell-free extract, a cell or an animal.

28. The method of claim 26, wherein the difference is a surrogate marker.

29. A method for treating a bone development disorder in an animal comprising transferring the nucleic acid sequence of claim 1 into a somatic cell of an animal suffering from a bone development disorder.

30. The method of claim 29, wherein the animal is a human or a bird.

31. A method for treating a bone development disorder in an animal comprising transferring the nucleic acid sequence of claim 1 into a germ-line cell of an animal suffering from a bone development disorder.

32. The method of claim 31, wherein the animal is a human or a bird.

33. A method of altering bone development in a host comprising administering the amino acid sequence of claim 3 to a somatic cell of a host suffering from a bone development disorder.

34. The method of claim 33, wherein the host is a human or a bird.

35. A method of altering bone development in a host comprising administering the amino acid sequence of claim 3 to a germ-line cell in a host suffering from a bone development disorder.

36. The method of claim 35, wherein the animal is a human or a bird.

37. A method of treating osteoporosis comprising administering the amino acid sequence of claim 3 to a patient in need thereof.

38. The method of claim 37, wherein the patient is a human or a bird.

39. A method of treating osteoporosis comprising administering the extracellular domain of the amino acid sequence of claim 3 to a patient in need thereof.

40. The method of claim 39, wherein the patient is a human or a bird.

41. A method of treating osteoporosis comprising administering the intracellular domain of the amino acid sequence of claim 3 to a patient in need thereof.

42. The method of claim 41, wherein the patient is a human or a bird.

43. A method for treating bone development disorders comprising administering a molecule that binds to the nucleic acid sequence of claim 1 to a patient in need thereof.

44. The method of claim 43, wherein the patient is a human or a bird.

45. A method for treating bone development disorders comprising administering an antibody to a patient in need thereof, wherein the antibody is to the amino acid sequence of claim 3.

46. A method for diagnostic screening for a genetic predisposition to a bone development disorder comprising screening a sample from a patient with a nucleotide sequence derived from the genomic or cDNA nucleic acid sequence of HBM or Zmax1.

47. A diagnostic assay for bone development disorders comprising an antibody to the HBM protein.

48. A method for identifying a genetic predisposition to bone development disorders comprising performing a haplotype analysis using the nucleic acid sequence of claim 1 or the nucleic acid of SEQ ID NO: 1.

49. A method of expressing the HBM protein in bone tissue comprising constructing an expression vector comprising a promoter that directs expression in bone tissue operably linked to the nucleic acid sequence of claim 1.

50. The method of claim 49, wherein the promoter that directs expression in bone is an osteocalcin promoter, a bone sialoprotein promoter or an AML-3 promoter.

51. A bacterial artificial chromosome having the nucleic acid sequence of SEQ ID NO: 5, 6, 7, 8, 9, 10 or 11.

52. A method for amplifying a nucleotide polymorphism in the Zmax1 gene comprising using the bacterial artificial chromosome of claim 51.

53. A method for amplifying a nucleotide polymorphism in the HBM gene comprising using the bacterial artificial chromosome of claim 51.

54. A method for identifying a regulatory element of a HBM gene comprising using the bacterial artificial chromosome of claim 1 or claim 51.

55. An isolated nucleic acid sequence comprising at least 15 contiguous nucleotides of SEQ ID NO: 2, wherein said 15 contiguous nucleotides comprise position 582 of SEQ ID NO: 2.

56. The isolated nucleic acid sequence of claim 55 that is DNA.

57. The isolated nucleic acid sequence of claim 55 that is RNA.

58. A replicative cloning vector comprising the nucleic acid sequence of claim 55 and a replicon operative in a host cell.

59. An isolated host cell transformed with the replicative cloning vector of claim 58.

60. An expression vector comprising the nucleic acid sequence of claim 55 operably linked to a transcription regulatory region.

61. An isolated host cell transformed with the expression vector of claim 60.

62. An isolated nucleic acid sequence comprising at least 15 contiguous nucleotides of SEQ ID NO: 2, wherein said 15 contiguous nucleotides comprise position 582 of SEQ ID NO: 2, and which encodes for an amino acid sequence including a valine corresponding to valine at position 171 of SEQ ID NO: 4 and wherein valine substitution is associated with a high bone mass phenotype.

63. The nucleic acid sequence of claim 62 which is DNA.

64. An isolated nucleic acid segment of at least 15 contiguous nucleotides including a polymorphic site from the nucleic acid sequence of SEQ ID NO: 2 in which G at position 582 is replaced by T, and sequences complementary thereto.

65. The isolated nucleic acid segment of claim 64, wherein said complementary sequence is the reverse complement.

66. The isolated nucleic acid segment of claim 65, wherein said reverse complementary sequence is mRNA.

67. The isolated nucleic acid segment of claim 64 that is DNA.

68. The isolated nucleic acid segment of claim 64 that is cDNA.

69. The isolated nucleic acid segment of claim 65 that is RNA.

70. An isolated nucleic acid segment of at least 15 contiguous nucleotides including a single nucleotide polymorphic site from an exon sequence selected from the group consisting of:
SEQ ID NO: 9 wherein nucleotide 69169 is replaced by A,
SEQ ID NO: 9 wherein nucleotide 27402 is replaced by G,
SEQ ID NO: 9 wherein nucleotide 27841 is replaced by C,
SEQ ID NO: 9 wherein nucleotide 35600 is replaced by G,
SEQ ID NO: 9 wherein nucleotide 45619 is replaced by A,
SEQ ID NO: 9 wherein nucleotide 46018 is replaced by G,
SEQ ID NO: 9 wherein nucleotide 46093 is replaced by G,
SEQ ID NO: 9 wherein nucleotide 46190 is replaced by G,
SEQ ID NO: 9 wherein nucleotide 50993 is replaced by C,
SEQ ID NO: 9 wherein nucleotide 51124 is replaced by T,
SEQ ID NO: 9 wherein nucleotide 55461 is replaced by T,
SEQ ID NO: 9 wherein nucleotide 63645 is replaced by A,
SEQ ID NO: 9 wherein nucleotide 63646 is replaced by C,
SEQ ID NO: 9 wherein nucleotide 24809 is replaced by G,
SEQ ID NO: 9 wherein nucleotide 27837 is replaced by C,
SEQ ID NO: 9 wherein nucleotide 31485 is replaced by T,
SEQ ID NO: 9 wherein nucleotide 31683 is replaced by G,
SEQ ID NO: 9 wherein nucleotide 24808 is replaced by G,
SEQ ID NO: 8 wherein nucleotide 31340 is replaced by C,
SEQ ID NO: 8 wherein nucleotide 32538 is replaced by G,
SEQ ID NO: 8 wherein nucleotide 13224 is replaced by G,
SEQ ID NO: 8 wherein nucleotide 21119 is replaced by A,
SEQ ID NO: 8 wherein nucleotide 30497 is replaced by A,
SEQ ID NO: 9 wherein nucleotide 24811 is replaced by C,
SEQ ID NO: 9 wherein nucleotide 68280 is replaced by A, and sequences complementary thereto.

71. The isolated nucleic acid segment of claim 70, wherein nucleotide 21119 of said exon sequence of SEQ ID NO: 8 is replaced by A.

72. The isolated nucleic acid segment of claim 70 that is DNA.

73. The isolated nucleic acid segment of claim 70 that is RNA.

74. The isolated nucleic acid segment of claim 64 or claim 70 which is a probe or a primer.

75. A method of identifying a molecule involved in bone modulation comprising identifying a molecule that binds to or that inhibits binding of a molecule to a protein involved in focal adhesion signaling.

76. The method of claim 75, wherein the molecule involved in focal adhesion signaling binds to a protein selected from the group consisting of: SEQ ID NO: 87-109.

77. The method of claim 75, wherein the molecule involved in focal adhesion signaling binds to a protein selected from the group consisting of: SEQ ID NO:90, SEQ ID NO:93, SEQ ID NO:94, SEQ ID NO:99 and SEQ ID NO: 102.

78. A method of modulating bone density in a subject by administering an agent that regulates a nucleic acid or polypeptide encoded thereby involved in focal adhesion signaling.

79. The method of claim 78, wherein the nucleic acid comprises a nucleic acid selected from the group consisting of: SEQ ID NOS: 63-86.

80. The method of claim 78, wherein the nucleic acid comprises SEQ ID NO: 66, SEQ ID NO: 71, SEQ ID NO: 77 or SEQ ID NO: 79.

81. The method of claim 78, wherein the polypeptide is selected from the group consisting of: SEQ ID NOS: 87-109.

82. The method of claim 78, wherein the polypeptide is SEQ ID NO:90, SEQ ID NO:93, SEQ ID NO:94, SEQ ID NO:99 or SEQ ID NO:102.

83. A nucleic acid comprising SEQ ID NO: 66, SEQ ID NO: 71, SEQ ID NO: 77 or SEQ ID NO: 79.

84. A nucleic acid of claim 83, wherein the nucleic acid is RNA or DNA.

85. A replicative cloning vector comprising a nucleic acid of claim 83 and a replicon operative in a host cell.

86. An isolated host cell transformed with the replicative cloning vector of claim 85.

87. An expression vector comprising the nucleic acid sequence of claim 83.

88. An isolated host cell transformed with the expression vector of claim 87.

89. A polypeptide comprising SEQ ID NO:90, SEQ ID NO:93, SEQ ID NO:94, SEQ ID NO:99 or SEQ ID NO: 102.

90. A nucleic acid encoding a polypeptide selected from the group consisting of SEQ ID NO:90, SEQ ID NO:93, SEQ ID NO:94, SEQ ID NO:99 or SEQ ID NO: 102.

91. A method of treating bone development disorders comprising the step of administering an agent which modulates a nucleic acid or a polypeptide involved in focal adhesion signaling.

92. The method of claim 91, wherein the nucleic acid modulated by the agent is selected from any one of SEQ ID NOS: 63-86.

93. The method of claim 91, wherein the polypeptide modulated by the agent is selected from any one of SEQ ID NOS: 87-109.

94. A method of testing a compound for bone modulation activity of Zmax1 or a Zmax1 mutant comprising:
(a) immobilizing a Zmax1 protein, or a Zmax1 mutant protein, or a portion of a Zmax1 protein or a portion of a Zmax1 mutant protein to a substrate;
(b) exposing a compound to said Zmax1 protein or portion thereof, or to said Zmax1 mutant protein or portion thereof;
(c) determining whether said compound bound to said Zmax1 protein or to said Zmax1 mutant protein; and
(d) administering said compound to a host and determining whether administration of said protein modulates bone mass, if said compound bound to said Zmax1 protein or said Zmax1 mutant protein.

95. The method of claim 94, wherein the compound is a protein.

96. A method for identifying proteins involved in Zmax1 signaling that modulates bone mass comprising:
(a) preparing a bait protein comprising operably linking a nucleic acid encoding Zmax1 or a biologically active portion of Zmax1 to a DNA binding domain of a yeast transcription factor;
(b) preparing a prey protein comprising operably linking a nucleic acid encoding a prety protein to a DNA binding domain of a yeast transcription factor;
(c) expressing said bait protein in a bate yeast and expressing said prety protein in a prey yeast;
(d) mixing said bait yeast and said prey yeast;
(e) determining whether the prey yeast mated to the bait yeast and given yeast mating, analyzing whether modulation of the prey protein modulates bone mass.

97. An isolated nucleic acid comprising SEQ ID NO: 1 or a biologically active fragment of SEQ ID NO: 1, wherein a nucleotide of SEQ ID NO: 1 which encodes YWTD motif is mutated and said mutation cuases an altered bone mass phenotype in a host when SEQ ID NO: 1 or said biologically active fragment of SEQ ID NO: 1 is epxressed in said host.

98. A replicative cloning vector comprising the nucleic acid of claim 97.

99. An isolated host cell transformed with the replicative cloning vector of claim 98.

100. A transgenic animal which expresses a mutated Zmax1, wherein said mutated Zmax1 produces an altered bone mass when expressed in said animal.

101. A method for treating a bone development disorder in an animal comprising transferring a nucleic acid comprising SEQ ID NO: 1 into a somatic cell of an animal suffering from said bond development disorder.

102. A method for treating a bone development disorder in an animal comprising transferring a nucleic acid comprising SEQ ID NO: 1 into a germ-line cell of an animal suffering from a bone development disorder.

103. A method of modulating bone development in a host comprising administering an amino acid sequence comprising SEQ ID NO:3 or a biologically active fragment thereof to a somatic cell of a host suffering from a bone development disorder.

104. An isolated high bone mass (HBM) polynucleotide comprising a nucleic acid selected from the group consisting of:
(a) a nucleic acid having SEQ ID NO: 2;
(b) a nucleic acid encoding a polypeptide of SEQ ID NO: 4;
(c) a nucleic acid amplified from a mammalian library using primers which hybridize to loci within the nucleic acid of SEQ ID NO: 2 or a nucleic acid encoding a polypeptide of SEQ ID NO: 4;
(d) a nucleic acid which hybridizes to the nucleic acid of SEQ ID NO: 2;
(e) a nucleic acid which is complementary to a nucleic acid of (a), (b), (c), and (d);
(f) a nucleic acid comprising at least 15 contiguous nucleotides which comprise position 582 of SEQ ID NO: 2 from a polynucleotide of (a), (b), (c), (d), and (e); and
wherein said isolated HBM polynucleotide encodes a protein or polypeptide, which when administered to a subject modulates bone mass.

105. The polynucleotide of claim 104, wherein the nucleic acid comprises at least 100 contiguous nucleotides from a polynucleotide of (a), (b), (c), (d) or (e).

106. A method for testing a substance as a therapeutic agent for bone modulation in a host comprising administering a nucleic acid comprising SEQ ID NO:1 to said host, and assessing whether bone modulation occurs in said host.

107. A method for expressing Zmax1 protein in bone tissue comprising constructing an expression vector comprising a promoter that directs expression in bone tissue operably linked a nucleic acid comprising SEQ ID NO: 1 or a nucleic acid encoding a biologically active fragment of SEQ ID NO: 1.

108. The method of claim 107, wherein the promoter is an osteocalcin promoter, a bone sialoprotein promoter of an AML-3 promoter.

109. The method of claim 108, wherein the promoter is the osteocalcin promoter and the bone tissue is osteoblasts.

110. A method of modulating bone mass in an animal comprising the step of administering a nucleic acid which is antisense to SEQ ID NO: 1 or to a portion of SEQ ID NO: 1 in an amount which modulates bone mass.

111. A method of studying bone disorders comprising preparing a transgenic animal which expresses Zmax1; regulating said Zmax1; and determining impact of said Zmax1 regulation on bone development in said mammal.

112. A transgenic animal which expresses a mutated Zmax1, wherein said mutated Zmax1 produces an altered bone mass when expressed in said animal.

113. A method of testing a compound as a bone modulating compound by administering said compound to said transgenic animal of claim 112 and determining whether said compound modulated bone mass in said animal.

114. An isolated high bone mass (HBM) polynucleotide of claim 104, wherein the nucleic acid is a nucleic acid of (a) or (b) or a nucleic acid complementary thereto.

115. An isolated nucleic acid sequence of claim 55, wherein said 15 contiguous nucleotides comprises positions 581, 582 and 583 of SEQ ID NO: 2.

116. An isolated nucleic acid sequence comprising at least 15 contiguous nucleotides of SEQ ID NO: 2, wherein said 15 contiguous nucleotides comprise nucleotides which encode for an amino acid substitution at position 171 of SEQ ID NO: 4; which substitution is associated with a high bone mass phenotype.

117. An isolated high bone mass (HBM) polynucleotide comprising a nucleic acid selected from the group consisting of:
(a) a nucleic acid having SEQ ID NO: 2;
(b) a nucleic acid encoding a polypeptide of SEQ ID NO: 4;
(c) a nucleic acid which is complementary to a nucleic acid of (a), (b); and (d) a nucleic acid comprising at least 15 contiguous nucleotides which comprise position 582 of SEQ ID NO: 2 from a polynucleotide of (a), (b), and (c).
